(19) United States
(12) Patent Application Publication
(10) Pub. No.: US 2003/0175961 A1
(43) Pub. Date: Sep. 18, 2003

(54) Immortal micorvascular endothelial cells and uses thereof

(76) Inventor: G. Scott Herron, La Honda, CA (US)

Correspondence Address: Lisa A. Amii, Morrison & Foerster LLP, 755 Page Mill Road, Palo Alto, CA, 94304-1018, US

(21) Appl. No.: 10/086,118

(22) Filed: Feb. 26, 2002

(51) Int. Cl. C12Q 1/00; A61K 48/00; C12N 5/08

(52) U.S. Cl. 435/372; 424/93.21; 435/4

(57) ABSTRACT

The present invention relates to telomerase-immortalized microvascular endothelial cells that form microvascular structures in vitro and in vivo. The telomerized cells have normal karyotype and demonstrate resistance to apoptosis. Methods for producing the cells and specific applications of cell compositions are provided.

### RELATED APPLICATIONS

This application is a continuation-in-part of a U.S. patent application titled, “In Vivo Assay for Anti Angiogenic Compounds,” serial no. XXX, pending, filed on Feb. 26, 2002, which is a non-provisional application converted from U.S. Provisional Application Serial No. 60/271,778, filed Feb. 27, 2001, which claims priority to WO 00/56898, filed Mar. 24, 2000, which is based on U.S. Provisional Application Serial No. 60/126,015, filed Mar. 24, 1999.

### FIELD OF THE INVENTION

The present invention relates to immortal microvascular endothelial cells having normal karyotype that demonstrate resistance to apoptosis, methods for producing said cells, and methods of use thereof. In particular, the immortal microvascular endothelial cells may be used in an assay for screening compounds to identify modulators of angiogenesis both in vitro and in vivo.

### BACKGROUND OF THE INVENTION

Emerging evidence suggests that apoptosis and the cell cycle are closely linked and use parts of the same molecular machinery (Meikamatz W, Schlegel R. Apoptosis and the cell cycle. Journal of Cellular Biochemistry 1995, 58(2):160-74; King K. L., Cidlowski J. A. Cell cycle and apoptosis: common pathways to life and death. Journal of Cellular Biochemistry 1995, 58(2): 175-80; Kasten M, Giordano A. pRb and the Cdks in apoptosis and the cell cycle. Cell Death and Differentiation 1998, Review: 132-140). Cells progressing through the cell cycle become more susceptible to apoptosis versus quiescent cells but interestingly, cell cycle arrest in late GI or S phase potentiates apoptosis. Cell cycle checkpoint proteins (e.g. p53, pRB and cyclin dependent kinase inhibitors, p21 and p27) are involved in making cell fate decisions of apoptosis or cycle arrest but precise mechanisms remain unclear (Evan G, Littlewood T. A matter of life and cell death. Science 1998, 281(5381):1317-22). It is known that unrepaired DNA and chromosomal damage triggers apoptotic induction justifying these checkpoint proteins as “guardians of the genome” (Lane D. P. Cancer. p53, guardian of the genome Nature 1992, 358(6381):15-6).

Chromosomal damage in the form of telomeric DNA shortening during cell division may serve as a “biological clock” that triggers replicative senescence. Cell cycle arrest at senescence is a complex and as yet poorly defined process that involves genetic programming much like the differentiated phenotype. Telomeric DNA shortens at a certain rate during each cell division due to the inability of standard DNA polymerases to synthesize DNA at the ends of chromosomes. Once a certain length is reached, a sensor determines that it's time for the cell to senesce and stop dividing. (Harley C, Vaziri H, Counter C, et al.) The telomere hypothesis of cellular aging. Exp Gerontol 1992, 27:375-382; Sedivy J M. Can ends justify the means?: telomeres and the mechanisms of replicative senescence and immortalization in mammalian cells. Proceedings of the National Academy of Sciences of the United States of America 1998, 95(16):9078-81). There are many pathways that lead to the final common state of replicative senescence but DNA damage is recognized as a major path involving p53-mediated G1 arrest (Di Leonardo A, Linke S P, Clarkin K, et al. DNA damage triggers a prolonged p53 -dependent G1 arrest and long-term induction of Cip1 in normal human fibroblasts. Genes and Development 1994, 8(21):2540-51). The state of replicative senescence is considered an “activated” state by many investigators, particularly with regards to the expression of genes involved in extracellular matrix metabolism (West M D. The cellular and molecular biology of skin aging. Archives of Dermatology 1994, 130(1):87-95; Campisi J, Dimri G P, Nehlin J O, et al. Coming of age in culture. Experimental Gerontology 1996, 31(1-2):7-12).

During angiogenesis, EC proliferation occurs in an area proximal to the tip of new vessels and these vessels represent sprouting postcapillary venules (Folkman J, Bream H. Angiogenesis and inflammation. In. (second 4 ed.) (J I Gallin L G, and R. Snyderman, ed. New York: Raven Press Ltd., 1992. In Inflammation: Basic Principles and Clinical Correlates). The EC cell cycle can be arrested by three main mechanisms: 1) Growth factor removal; 2) Extracellular matrix signaling a “nonpermissive” environment (Ingber D E. Extracellular matrix as a solid-state regulator in angiogenesis: Identification of new targets for anti-cancer therapy. Seminars in Cancer Biology 1992, 3(2):57-63) and 3) Contact inhibition.

An emerging theme in the control of EC proliferation is that growth factor and ECM signaling are tightly coupled via matricellular proteins such that endogenous angiostatic factors appear to sequester growth factors, block receptor activation and even induce EC apoptosis (Bornstein P. Diversity of function is inherent in matricellular proteins: an appraisal of thrombospondin 1. Journal of Cell Biology 1995, 130(3):503-6; Sage E H. Pieces of eight bioactive fragments of extracellular proteins as regulators of angiogenesis. Trends Cells Biol 1997, 7:182 -186; Kupprion C, Motamed K, Sage E H. SPARC (BM-40, osteonectin) inhibits the mitogenic effect of vascular endothelial growth factor on microvascular endothelial cells. Journal of Biological Chemistry 1998, 273(45):29635-40; Lucas R, Holmgren L, Garcia I, et al. Multiple Forms of Angiostatin Induce Apoptosis in Endothelial Cells. Blood 1998, 92(12):4730-4741). EC cell-cell signaling is an equally strong regulator of EC proliferation in vitro and signaling through intercellular contacts clearly regulates EC cell cycle machinery (Yoshizumi M, Lee W S, Hsieh C M, et al. Disappearance of cyclin A correlates with permanent withdrawal of cardiomyocytes from the cell cycle in human and rat hearts. Journal of Clinical Investigation 1995, 95(5):2275-80; Nakamura Y. Cleaning up on beta-catenin [news]. Nature Medicine 1997, 3(5):499-500).

The mechanisms of EC cell cycle arrest and induction of the quiescent state by the above three events are different but reversible. Clearly none represent replicative senescence. Thus, while many angiostatic factors work by GI growth arrest and delay of entry into S phase (Funk SEea). Differential effects of SPARC and cationic SPRC peptides on DNA synthesis by endothelial cells and fibroblasts. J. Cell Physiol 1993, 154:53-63; Gupta S K, Singh J P. Inhibition of endothelial cell proliferation by platelet factor-4 involves a unique action on S phase progression. Journal of Cell Biology 1994, 127(4):1121-7; Irnamura T, Oka S, Tanahashi T, et al. Cell cycle-dependent nuclear localization of exogenously added fibroblast growth factor-I in BALB/c 3T3 and human vascular endothelial cells. Experimental Cell Research 1994, 215(2):363-72; Baldin V, Roman A M, Bosc-Bierne I, et al. Translocation of bFGF to the nucleus is G1 phase cell cycle specific in bovine aortic endothelial cells. Embo Journal 1990, 9(5): 1511-7; Hori A, Ikeyama S, Sudo K. Suppression of cyclin D I mRNA expression by the angiogenesis inhibitor TNP-470 (AGM-1470) in vascular endothelial cells. Biochemical and Biophysical Research Communications 1994, 204(3):1067-73; Abe J, Zhou W, Takuwa N, et al. A fumagillin derivative angiogenesis inhibitor, AGM-1470, inhibits activation of cyclin-dependent kinases and phosphorylation of retinoblastoma gene product but not protein tyrosyl phosphorylation or protooncogene expression in vascular endothelial cells. Cancer Research 1994, 54(13):3407-12) molecular mechanism(s) responsible for EC senescence are not known.

The adult endothelium in vivo is remarkably quiescent and endothelial cells (EC) divide very slowly unless activated in some way. Isolation and culture of EC result in a semi-activated state in which cells retain some specialized characteristics but lose others (Cines D B. Glycoprotein IIb/IIIa antagonists: potential induction and detection of drug-dependent antiplatelet antibodies. American Heart Journal 1998, 135(5 Pt 2 Su):S152-9). All human EC appear to retain the ability to divide many times in vitro but their continued survival depends on a variety of different factors and conditions (Bicknell R. Endothelial Cell Culture. (Bicknell R, ed. Oxford: Cambridge University Press, 1996). When EC become activated via inflammatory cytokines, oxidative stress, or other pathologic insults, many different vasoprotective genes are induced. Some of these include; Bcl-2 family members, A20, MnSOD, 70-kDa HSP, heme oxygenase-1, and VEGF. Without the induction of such genes EC survival and replication would not match the loss of EC during states of inflammation.

Like all somatic cells, human EC undergo replicative senescence after a finite number of divisions which varies between 20 and 50 population doublings (PD) depending on the tissue of origin and culture conditions. Human vascular endothelial cells (HUVECs) appear to respond to autocrine production of IL-1a by undergoing senescence (Maier J A, Voulalas P, Roeder D, et al. Extension of the lifespan of human endothelial cells by an interleukin-I alpha antisense oligomer. Science 1990, 249(4976):1570-4; Maier J A, Statuto M, Ragnotti G. Endogenous interleukin 1 alpha must be transported to the nucleus to exert its activity in human endothelial cells. Molecular and Cellular Biology 1994, 14(3): 1845-51). More recently, VEGF was found to both delay the onset of replicative senescence in HDMEC and reverse senescence of HDMEC when added to cultures grown without VEGF (Watanabe Y, Lee S W, Detmar M, et al. Vascular permeability factor/vascular endothelial growth factor (VPF/VEGF) delays and induces escape from senescence in human dermal microvascular endothelial cells. Oncogene 1997, 14(17):2025-32). Senescent HDMEC expressed high levels of both p16 and p21 CDK inhibitors relative to VEGF-treated HDMEC and VEGF withdrawal increased p16 with little effect on p21. The data suggests that replicative senescence in HDMEC is associated with GI growth arrest involving p16.

VEGF is known to be an important survival factor for all EC and its absence has dramatically negative effects on vasculogenesis, angiogenesis and vascular remodeling (Risau W. Mechanisms of angiogenesis. Nature 1997, 386:671-674). VEGF signaling pathways depend on a set of receptor tyrosine kinases (nearly) unique to EC; flt-1/VEGFR1, flk-1/KDR/VEGFR2 and TIE-2. VEGFR2 downregulation is thought to be important in EC senescence and death in vitro (Hewett P W, Murray J C. Coexpression of fit-1, fit-4 and KDR in freshly isolated and cultured human endothelial cells. Biochemical and Biophysical Research Communications 1996, 221(3):697-702). It is now well established that vascular regression in vivo can be induced by VEGF withdrawal in the presence of elevated angiopoietin-2 expression (Maisonpierre P C, Suri C, Jones P F, et al. Angiopoietin 2, a natural antagonist for Tie2 that disrupts in vivo angiogenesis, Science 1997, 277(5322):55-60) but molecular mechanisms are unclear.

The “survival role” of VEGF receptor signaling in human EC is becoming more apparent. Human Herpes Virus 8 in Kaposi's sarcoma (KSHV) can infect both primary bone marrow microvascular EC (BMEC) and HUVEC producing spindle cells which bypassed replicative senescence; however, less than 5% of the cells actually contained the KSHV viral genome and these cells exhibited a fully transformed phenotype (Flore O, Rafii S, Ely S, et al. Transformation of primary human endothelial cells by Kaposi's sarcoma-associated herpesvirus. Nature 1998, 394(6693):588-92). The other 95% of uninfected primary EC also bypassed replicative senescence as “bystanders” in mass cultures because VEGFR2 expression was induced by the paracrine effect of KSHV-infected EC.

In the skin, both epidermal and mesenchymal cells express VEGF; however, VEGF is also an autocrine factor synthesized and secreted directly by HDMEC in response to hypoxia (Detmar M, Brown L F, Berse B, et al. Hypoxia regulates the expression of vascular permeability factor/vascular endothelial growth factor (VPF/VEGF) and its receptors in human skin. Journal of Investigative Dermatology 1997, 108(3):263-8).

Cell proliferation and survival are critical parameters useful for screening compounds for treatment of various disorders, including tumors and other proliferative disorders. Compounds that are selected for their ability to inhibit cell proliferation can act to (1) inhibit mitogenesis, (2) inhibit angiogenesis, or (3) activate the complement pathway and the associated killer cells.

Angiogenesis is normally observed in wound healing, fetal and embryonal development and formation of the corpus luteum, endometrium, and placenta. The control of angiogenesis is a highly regulated system of angiogenic stimulators and inhibitors. Thus, angiogenesis is a critical component of the body's normal physiology, especially during wound healing.

In addition, the control of angiogenesis has been found to be altered in certain disease states, and, in many cases, the pathological damage associated with the disease is related to uncontrolled angiogenesis. It also has a detrimental aspect, for example, when blood vessels multiply and enhance growth and metastasis of tumors. Aberrant angiogenesis is also associated with numerous disorders, including rheumatoid arthritis, where blood vessels invade the joint and destroy cartilage, and numerous ophthalmologic pathologies, such as diabetic retinopathies in which new capillaries invade the vitreous, bleed and cause blindness, and macular degeneration, prostate cancer and Kaposi's carcinoma. Angiogenesis is essential to tumor development and growth. Prevention of angiogenesis can inhibit solid tumor growth.

Compounds that have anti-angiogenic activity can be used, for example, as anti-tumor agents and for the treatment of ophthalmic disorders, particularly involving the retina and vitreous humor, and for hyperproliferative dermatological disorders, such as psoriasis, that have an angiogenic component. Thus, compounds that enhance angiogenesis and compounds that inhibit angiogenesis are being sought.

This has led to a search for specific inhibitors of endothelial cell growth. As a result, there is an interest in measuring proliferation of endothelial cells under inhibitory and stimulatory conditions as screens for discovery of inhibitors (or alternatively stimulators) of angiogenesis. Direct assessment of cell numbers, either microscopically or by particle counter is time consuming and not amenable for high throughput screening. Consequently, direct assessment has been replaced by indirect methods, such as by packed cell volume, by chemical determination of a cellular component, for example, protein or deoxyribonucleic acid, or by uptake of a chromogenic dye such as neutral red. These methods can be laborious when handling large numbers of cultures, and also inaccurate at low cell densities. For high throughput screening protocols it is necessary to rapidly and accurately measure low cell densities and/or relatively small changes in cell number over a large range of cell densities. Presently available protocols do not provide a means to do this and do not measure the end result of angiogenesis which is a change in the number of capillary blood vessels. Thus, there is a need for convenient, rapid and reproducible assays for identifying agents that modulate angiogenesis as well as agents that modulate cell proliferation.

There are a number of barriers to the development of rapid and reproducible human microvascular remodeling assay systems that the art has failed to address. These include cell variability, cell viability, and morphogenetic response measurement.

Cell variability: Primary microvascular endothelial cell (MEC) cultures isolated from human tissues represent mixtures of cells (i.e. mass cell cultures) derived from both arteriolar and venular microvessels 15-50 um in diameter, as well as, lymphatic vessels. When these cells are expanded in culture, a variable number of different cell populations will predominate according to purity of the cells, donor age and source, growth conditions and passage number. Primarily, it is this cell population variability that gives rise to markedly different responses to morphogenetic stimuli and resultant irreproducibility in standard in vitro angiogenic assay systems (e.g. 3D collagen and Matrigel).

Cell viability: Primary human MEC, as defined above (and further within), have finite lifespans in vitro which vary between 20 and 40 population doublings. Depending on culture conditions MEC will undergo either “culture senescence” or “replicative senescence” wherein cultures cannot be expanded any longer and stop dividing. Culture senescence is seen when suboptimal growth conditions result in growth factor unresponsiveness and/or apoptosis due to a variety of different conditions (e.g. pC02, p02, serum concentration, survival factor concentration, lack of flow, etc), whereas, MEC replicative senescence is observed when telomeric DNA shortens to such a degree that the senescence program is activated and cells also become growth factor unresponsive. It is this finite lifespan that limits the development of angiogenic assay systems because MEC survival cannot be maintained long enough to allow statistically significant and reproducible measurements of the process.

Morphogenetic response measurement: The formation of new capillaries in permissive extracellular matrices and tissues is complex and not well understood. Visualization of the angiogenic process requires image analysis of vessel formation inside these matrices. Rapid and efficient methods for quantifying this process either in vitro or in vivo are rudimentary and consist of primarily of micromorphometric measurements of vessel lumens in stained tissue sections. This process is slow and tedious. One reason for this is that human MEC have not been genetically “tagged” to stably express marker elements that can be tracked, traced or otherwise detected so as to produce images that can be digitally converted (e.g. by immunofluorescence microscopy) into usable information. Software programs measuring this process are not readily available or have not been developed.

The present invention, as herein below illustrates, provides new methods and compositions for overcoming these obstacles.

### SUMMARY OF THE INVENTION

The present invention provides methods for the generation of immortal microvascular endothelial cells, including immortal human dermal microvascular endothelial cells (HDMECs), having normal karyotype that demonstrate resistance to apoptosis. The immortal microvascular endothelial cells of the present invention are not transformed, and have no activated oncogenes (i.e., that result in malignant transformation). The cells have an essentially normal phenotype as compared to primary microvascular endothelial cells. These immortal cells were generated by the introduction of the human telomerase reverse transcriptase catalytic subunit gene (hTERT) into primary endothelial cells. Endothelial cells from other human tissue locations and other animal sources may also be produced by the methods of the present invention.

In one embodiment, the present invention includes a composition of immortal microvascular endothelial cells, where the cells of the compositions each contain a recombinant expression cassette encoding telomerase. The expression cassette can include a number of control elements. Typically the expression cassette contains at least a promoter operably linked to the telomerase coding sequence. The immortal microvascular endothelial cells of the present invention (a) have a normal karyotype, (b) are resistant to apoptosis relative to primary microvascular endothelial cells, and (c) are not transformed. Further, the immortal microvascular endothelial cells have an essentially normal phenotype relative to primary microvascular endothelial cells.

In a preferred embodiment of the present invention the immortal microvascular endothelial cells are derived from primary human dermal microvascular endothelial cells.

In still another preferred embodiment, the present invention provides immortal microvascular endothelial cells that incorporate a genetic label or tag. A further preferred embodiment of the present invention provides immortal microvascular endothelial cells that incorporate eGFP producing a uniform population of fluorescently labeled cells. One aspect of the present invention is a composition of microvascular endothelial cells that demonstrate superior survival characteristics both in vitro and in vivo relative to primary cells. More specifically such superior survival includes an extended cellular life span as well as resistance to apoptosis comparable to young primary human dermal microvascular endothelial cells.

In one aspect of the invention, the immortal microvascular endothelial cells can be used to generate xenograft mice. Such mice provide an angiogenesis model useful, for example, for screening therapeutic compounds.

Further, the immortal microvascular endothelial cells can be used to generate new blood vessels, reline the surfaces of existing vasculature, create new vasculature and vascular structures in subjects. Therapeutic uses of these cells include, treatment, for example, of atherosclerosis. The cells are also useful in methods of reversing vascular system inflammatory response.

In addition, the immortal microvascular endothelial cells of the present invention provide methods of treating tumors, e.g., administering immortal microvascular endothelial cells containing inducible genetic elements that code for anti-tumor and/or therapeutic compounds that would target occult sites of angiogenic activity.

Still a further aspect of the present invention provides in vivo human microvascular remodeling assay systems using eGFP-labeled immortalized microvascular endothelial cells which form fluorescent capillary blood vessels having vessel density which can be assessed by digital imaging. The in vivo assay may be implemented, e.g., to determine the effect of a pharmaceutically acceptable compound on angiogenesis.

Further, the immortal microvascular endothelial cells of the present invention can be created from cells derived from different human anatomic sites or can be created from animals, different animal anatomic sites, or from genetically modified (e.g. transgenic) animals. The immortal microvascular endothelial cells of the present invention can be obtained from a number of human and animal sources including, but not limited to, the following: normal neonatal foreskin, adult normal skin, and pediatric skin; as well as, adult and pediatric pathologic skin derived from patients with different cutaneous disease states (including but not limited to, scleroderma, psoriasis, Epidermolysis Bullosa, hemangiomas and other vascular proliferative lesions, skin tumors, vasculitic lesions, nonhealing wounds and wounds in different stages of healing).

The immortal microvascular endothelial cells of the present invention can be supplied as a commercial product that provides EC which are easy to grow, have a normal karyotype, display a consistent phenotype, are not transformed, and are immortal.

The immortal microvascular endothelial cells of the present invention also provide pharmacologic and toxicologic methods of screening and testing new drugs designed to modulate the growth of blood vessels in vivo using human EC (e.g., by incorporation of immortal microvascular endothelial cells into animal models of angiogenesis and vascular remodeling). The immortal microvascular endothelial cells of the present invention also provide a number of in vivo therapeutic strategies, including, but not limited to, the following: 1) replacement cells in disease states involving inadequate or dysfunctional proliferation/regression of host EC at the site of disease via transplantation; 2) gene transfer vehicles to express ectopic genes requiring vascular delivery in monogenetic diseases and autoimmune diseases; and 3) gene delivery vehicles to express ectopic genes (e.g., angiostatic factors; AS, ES, TSP, TIMPs) that would deter the proliferation and spread of occult malignant tumors during the early stages of tumor-induced angiogenesis.

The immortal microvascular endothelial cells of the present invention have characteristics that are useful in the design of vascular model systems and therapeutic strategies for treating age-related diseases of the vasculature.

These and other embodiments of the present invention will readily occur to those of ordinary skill in the art in view of the disclosure herein.

Citation of the documents herein is not intended as an admission that any of the foregoing is pertinent prior art. All statements as to the date or representation as to the contents of these documents is based on the information available to the applicants and does not constitute any admission as to the correctness of the dates or contents of these documents. Further, all documents referred to throughout this application are hereby incorporated in their entirety by reference herein.

### BRIEF DESCRIPTION OF THE DRAWINGS

FIGS. 1A to 1F are photomicrographs of transfection results obtained using the LZRS-eGFP vector in HDMEC.

FIG. 2 presents a graph that shows an inverse correlation of reporter gene expression efficiency and passage number.

FIGS. 3A and 3B present results of transfection of HDMEC with the LZRS-vector carrying coding sequences for the CD34 protein.

FIGS. 4A to 4D present the results of fluorescence activated cell sorting that was carried out on transfected cells.

FIG. 5 presents a vector construct containing hTERT coding sequences.

FIG. 6 presents the results of RT-PCR analysis to test for the presence of the hTERT transgene.

FIG. 7 presents Southern blotting results where the hybridization was carried out with a biotinylated telomere probe.

FIG. 8 presents karyotypic analysis for hTERT (+) HDMEC.

FIGS. 9A and 9B presents the results of FACs quantification of the expression of Apo2.7, an early and specific mitochondrial-associated apoptotic marker.

FIGS. 10A and 10B presents the results of the effect of population doubling number (PD#) on absolute baseline of (unstimulated) values of apoptosis using both nuclear and mitochondrial apoptotic analysis.

FIGS. 11A and 11B presents the results of stimulated apoptosis analyses, as described for FIGS. 10A and 10B, that were repeated on separate cells.

FIG. 12 presents the results of a series of immunoblots using polyclonal anti-human ES antiserum (Fibroblast and microvascular endothelial cell endostatin).

FIG. 13 presents the results of a series of immunoblots using polyclonal anti-human ES antiserum (microvascular endothelial cell endostatin).

FIG. 14 presents data demonstrating the relative apoptotic resistance of hTERT(+)HDMEC induced by permissive 3D collagen matrix exposure.

FIG. 15 presents data showing the utility of using eGFP-labeled hTERT(+)HDMEC for tracking morphogenetic patterns of cells forming microvascular structures in vitro.

FIG. 16 presents data demonstrating the utility of using eGFP-labeled hTERT(+)HDMEC in 3D Matrigel for visualizing microvessel formation.

FIG. 17 presents an example of how hTERT(+)HDMEC can be used to test the angiostatic characteristics of cyclo-oxygenase inhibitor compounds.

FIG. 18 presents the results of a human in vivo microvascular remodeling assay system in which SCID-mice are implanted with Matrigel mixed with hTERT(+)HDMEC. Results show creation of SCID-human chimeric blood vessels.

FIG. 19 presents data demonstrating the presence of fluorescent human blood vessels in the SCID mouse and the superiority of hTERT(+)HDMEC versus primary cells.

FIG. 20 presents data showing how human Type 4 collagen immunoreactivity is used to estimate human microvascular density. Results demonstrate the superiority of hTERT(+)HDMEC at in vivo microvessel formation.

FIG. 21 presents immuno-micromorphometry data in graphic format showing quantification of microvessel density and demonstration of the superiority of hTERT(+)HDMEC at in vivo microvessel formation.

FIG. 22 presents data demonstrating the specificity of hTERT(+)HDMEC at in vivo microvessel formation. HT1080 fibrosarcoma cells and primary dermal fibroblasts exhibit no human Type 4 collagen immunoreactivity.

FIG. 23 presents data demonstrating the specificity of hTERT(+)HDMEC at in vivo microvessel formation using eGFP-labeled HT1080 human fibrosarcoma cells, human embryonic kidney 293 cells and hTERT(+)HDMEC.

FIG. 24 presents data on the effect of phorbol ester (PMA) and anti-vitronectin receptor antibody (LM609) on eGFP-labeled hTERT(+)HDMEC using the in vivo microvascular remodeling assay system.

FIG. 25 presents data on microvessel density using anti-human Type 4 collagen immuno-reactivity comparing the effect of phorbol ester (PMA) and anti-vitronectin receptor antibody (LM609) on vessel formation in vivo.

FIG. 26 presents data on microvessel density using anti-human Type 4 collagen immuno-reactivity comparing the effect of b-FGF and VEGF on vessel formation in vivo.

FIG. 27 presents quantitative immuno-micromorphometry data in graphic format comparing the effects of b-FGF, VEGF, LM609 and PMA on microvessel density in vivo.

FIG. 28 shows the characterization of telomerase activity and fluorescence signal of eGFP-labeled primary HDMEC and telomerized cells. (A) Telomerase activity by TRAP protocol in two different primary parental HDMEC (non-eGFP labeled HDMEC, eGFP-labeled HDMEC-G) and their telomerized progeny (HDMEC-T, HDMEC-GT). Telomerized cells showed typical 6-nucleotide-DNA laddering at PD90 and PD28, respectively, whereas, little or no activity was observed in parental controls at PD25 and PD28. No TRAP activity was present in heat treated (HT, 65° C.×10 min) samples. (B) HDMEC-GT was sorted into a GFP(+) subpopulation by FACS. Two peaks in the GFP(+) population indicate some variability of fluorescence intensity among cells (insert). FAC-sorted HDMEC-GT maintained similar fluorescence signal patterns at PD80.

FIG. 29 shows the In vitro tubule formation in primary and telomerized HDMEC using 3D Matrigel. Phase contrast (A, C, E, G) and fluorescence (B, D, F, H) microscopy showed tubule formation was inversely correlated with in vitro aging of primary cells. Pre-senescent primary cells (HDMEC-G, PD38, A & B) exhibited no tubules and mid passage HDMEC-G (PD20, C & D) formed nonbranched, linear structures with diminished GFP fluorescence. HDMEC-GT (PD56, E-H) formed mature tubules with many branches and strong GFP signal (E, F, G, H). Bar: ˜20 μm.

FIG. 30 shows the in vivo tubule formation in SCID mice xenografted with HDMEC. (A) H&E staining, human type IV collagen immunofluorescence and GFP fluorescence signals in sections of Matrigel implants containing pre-senescent HDMEC-G (PD38) and HDMEC-GT (PD80) at two weeks after xenografting. Presence of vascular structures in both primary and telomerized implants is evident in H&E sections; however, only HDMEC-GT formed abundant capillary networks that were immunoreactive with anti-human type IV collagen IgG (col 4) and brightly GFP(+). Details of fluorescent vascular structures are enhanced by digital image analysis using the Moss Filter™ (Bin). Bar: ˜20 μm. (B). Graphic representation of human vessel density in Matrigel implants in vivo as a function of time after implantation using micromorphometry (i.e., counting the number of human type IV collagen immunoreactive annular structures per 5 random high power fields). HDMEC-GT at PD54 were directly compared with parental HDMEC-G cells at early (E; PD12), middle (M; PD20) and late (L; PD40) passages. Animals with replicate implants of each cell type were examined at 2 wk (black bars), 4 wk (white bars) and 6 wk (hatched bars) after implantation except for HDMEC-G at E passage, which had only a 2 week time point. The number of HDMEC-GT vessels was significantly different from HDMEC-G at M (*, p<0.01) and L passage (**, p<0.001). Averages and standard deviations are presented and each time point came from at least 3 independent experiments.

FIG. 31 shows the specificity of HDMEC-GT at forming in vivo tubules in SCID mice. Upper panels, eGFP-transduced HT1080 and 293 embryonic kidney tumor cells formed fluorescent tumor masses 2 weeks after implantation in Matrigel, whereas, HDMEC-GT formed microvascular networks only. Lower panels, H&E staining and immunofluorescence of Matrigel implanted HT1080 cells, human dermal fibroblasts and HDMEC-GT show human type IV collagen immunoreactive lumenal structures present only in HDMEC-GT. Bar: ˜20 μm.

FIG. 32 shows telomerized human microvessels communicate with host murine circulatory system. (A) Red blood cells (arrows) are visible within human type IV collagen immunoreactive lumenal structures derived from both young primary HDMEC-G (PD12) and telomerized HDMEC-T (PD70). Host vessel invasion of Matrigel implants is stimulated in the presence of FGF-2 (upper left panel); however, H & E staining, does not differentiate human from host vessels (middle and right upper panels). Human basement membrane collagen reproducibly reacts with human microvessels in Matrigel (middle and right lower panels). Bar: ˜10 μm. (B) Intravenous injection of red microspheres results in appearance of red tracer within eGFP(+) vessels. Dual scans using FITC (a) and rhodamine fluorescence (b) of the same image shows overlap of signals in some vasculature. Host vessels containing red tracer are present in the same field. In (c), FITC and rhodamine signals were overlaid (Metamorph, UIC) to simultaneously demonstrate the presence of tracer beads within eGFP(+) branched vessel. Bar: ˜20 μm.

FIG. 33 shows the effect of pro- and anti-angiogenic factors on HDMEC-GT derived microvessels in vivo. (A) Human type IV collagen immunoreactive vascular lumens two weeks after implantation in the presence of VEGF (2 μg/ml) or FGF-2 (150 ng/ml) demonstrates increased vessel density within grafts. Quantification by micromorphometry shows increased vessels for both growth factors but only FGF-2 reached statistical significance (* p<0.01). Bar: ˜20 μm. (B) Constitutive in vivo delivery of recombinant human endostatin (gel insert) via co-incubation of HDMEC-GT and endostatin cDNA-transfected 293 cells in Matrigel implants (HDMEC-GT+HEK293endo; b, d) shows decreased microvessel formation versus implants containing sham-transfected control cells (HDMEC-GT+HEK2931acZ; a, c) as demonstrated by both human type IV collagen staining (a, b) and binary images of eGFP fluorescence (c, d). Quantification by micromorphometry (left graph; n=6 different sections viewed) and total intensities extracted from binary images (Moss FilterM, right graph; n=6 different images for HEK2931acZ, n=8 different images for HEK293endo) shows inhibition is statistically significant (* p<0.001). Bar: ˜20 μm.

### DETAILED DESCRIPTION OF THE INVENTION

The practice of the present invention will employ, unless otherwise indicated, conventional methods of molecular biology, chemistry, biochemistry and pharmacology, within the skill of the art. Such techniques are explained fully in the literature. See, e.g., Remington 's Pharmaceutical Sciences, 19th Edition (Easton, Pennsylvania: Mack Publishing Company, 1995); Methods In Enzymology (S. Colowick and N. Kaplan, eds., Academic Press, Inc.); Wang, A. M., et al. in PCR Protocols: a Guide to Methods and Applications (M. A. Innis, et al., eds.) Academic Press (1990); Kawasaki, E. S., et al., in PCR Technology: Principles and Applications of DNA Amplification (H. A. Erlich, ed.) Stockton Press (1989); Hochuli, E., in Genetic Engineering, Principals and Practice, Vol. 12 (J. Stelow Ed.) Plenum, N.Y., pp. 87-98 (1990); Ausubel, F. M., et al., Current Protocols in Molecular Biology, John Wiley and Sons, Inc., Media Pa.; and, Sambrook, J., et al., In Molecular Cloning: A Laboratory Manual, Cold Spring Harbor Laboratory Press, Vol. 2 (1989).

All publications, patents and patent applications cited herein, whether supra or infra, are hereby incorporated by reference in their entirety.

As used in this specification and the appended claims, the singular forms “a,” “an” and “the” include plural references unless the content clearly dictates otherwise. Thus, for example, reference to “an antagonist” includes a mixture of two or more such agents.

Definitions

In describing the present invention, the following terms will be employed, and are intended to be defined as indicated below.

“Apoptosis” as used herein, refers to “programmed cell death.” A type of cell death, distinguished from necrosis, in which a program of cellular suicide is initiated involving both proteinase and nuclease activation. The apoptotic cell is identified by specific morphologic appearance of shrinkage, nuclear fragmentation and dysadherence, as well as, many molecular markers of mitochondrial death initiator release, activation of ICE proteinases (caspases) and degradation of multiple macromolecular targets (cell-cell and cell-matrix junctional components, cell cycle regulatory complexes and mitotic machinery).

“Normal karyotype” as used herein, refers to a normal karyotype of primary cells, typically for human cells, the normal complement of chromosomes is 46 (XX or XY) with a normal banding pattern as determined by staining, chromosome spreads, and microscopic evaluation.

“Normal phenotype” as used herein, refers to cells having essentially the same characteristics as untransformed, primary cells. Specific characteristics of human dermal microvascular endothelial cells, such as surface markers, morphology, tubule formation, etc., are discussed below.

“Untransformed” or “not transformed” as used herein, refers to cells that have not been transfected with an oncogene (for example, cellular or viral oncogenes) or where a cellular oncogene has not specifically been activated (as by, for example, integration of a viral sequence containing a promoter adjacent a cellular oncogene). Further, a cell that is not transformed has the characteristic that it does not grow in soft agar and does not form tumors or metastasize in experimental animals (Jiang X, Jimenez G, Chang E, et al. Telomerase expression in human somatic cells does not induce changes associated with a transformed phenotype. Nature Genetics 1999;21:1 11-114; Morales C, Holt S, Ouellette M, et al. Absence of cancer associated changes in human fibroblasts immortalized with telomerase. Nature Genetics 1999;21:115-1183).

“Telomere” as used herein refers to a terminal section of a eukaryotic chromosome, comprising about a few hundred base pairs, which has a specialized structure, and is involved in chromosomal replication and stability.

“Replicative senescence” as used herein refers to a cellular state wherein DNA replication is not being carried out and wherein DNA repair finctions are reduced or absent. Replicative senescence is associated with G Zero or GI growth arrest. Senescent cells typically show no telomerase activity and are resistant to apoptosis.

“hTERT” as used herein, refers to the human telomerase reverse transcriptase catalytic subunit and coding sequences thereof. Other telomerases may be used in the practice of the present invention. Further, coding sequences for hTERT may be obtained from a human source or synthetically prepared.

“Telomerase” as used herein, refers to a cellular enzymatic activity (for example, hTERT) that is capable of repairing and/or extending chromosomal telomeric sequences.

“Microvascular endothelial cells” as used herein, typically refers to a population of vascular cells derived from the skin 40-2000 um deep, representing 15-50 um in diameter blood vessels and lymphatic vessels comprising the papillary to deep reticular dermis which express marker proteins and differentiated functions associated nearly exclusively with endothelial cells of the microvasculature (Braverman, I., Cutaneous Microvasculature, Chapter 24 and Petzelbauer, P., Schechner, J S. and Pober, J S., Endothelium, Chapter 25. In: Fitzpatrick's Dermatology in General Medicine, 5th Edition, Freedberg, I., Eisen, A., Wolff, K., Austen, Y. F., Goldsmith, L A., Katz, S. and Fitzpatrick, T B., eds, McGraw-Hill, NY, 1999, pp.299-320). For example, the cell populations are typically mixed and can contain endothelial cells derived from: a) capillary loops (ascending and descending segments) of the papillary dermis (intrapapillary segments) and adnexal structures (sebaceous, eccrine glands and hair follicles); b) superficial horizontal dermal plexus (terminal arterioles, venous and arterial capillaries, postcapillary venules); c) interconnecting ascending arterioles and descending venules, and; d) deep horizontal vascular plexus. Primary human dermal microvascular endothelial cells (HDMEC) can be derived, for example, from neonatal foreskin.

“Inflammatory response” as used herein, refers to a nonspecific defensive reaction of the body to invasion by a foreign substance or organism that involves phagocytosis by white blood cells and is often accompanied by accumulation of pus and an increase in the local temperature.

“Atherosclerosis” as used herein, refers to deposition of lipid with proliferation of fibrous connective tissue cells in the inner walls of the arteries.

“Angiostatic Switch” as used herein, refers to the induction of endothelial cell (EC) apoptosis by angiostatic factors in actively growing blood vessels.

“Resists cell death” as used herein, refers to a first cell line that, when compared to another cell line(s) of the same type, does not enter senescence, apoptosis, or cell-cycle arrest.

“Finite replicative life” as used herein, refers to a finite number of times the DNA of a cell can be replicated, typically corresponds to a finite number of cell divisions that a cell may undergo.

“Immortal” as used herein, refers to cells that do not enter into “replicative senescence.” Typically, the cells are capable of dividing at least twice as many times as those from which they were derived (parental). Unlike transformed cells, immortal cells are supposed to maintain the normal phenotype, karyotype and finction of parental cells.

“Mitotic Clock” as used herein, refers to chromosomal damage in the form of telomeric DNA shortening during cell division may serve as a “biological clock” that triggers replicative senescence. Cell cycle arrest at senescence is a complex and as yet poorly defined process that involves genetic programming much like the differentiated phenotype (Harley C, Vaziri H, Counter C, et al. The telomere hypothesis of cellular aging. Exp Gerontol 1992, 27:375-382; Sedivy J M. Can Ends Justify The Means?: telomeres and the mechanisms of replicative senescence and immortalization in mammalian cells. Proceedings of the National Academy of Sciences of the United States of America 1998, 95(16):9078-81). There are many pathways that lead to the final common state of replicative senescence but DNA damage is recognized as a major path involving p53-mediated G1 arrest (Di Leonardo A, Linke S P, Clarkin K, et al. DNA damage triggers a prolonged p53 -dependent G1 arrest and long-term induction of Cip1 in normal human fibroblasts. Genes and Development 1994, 8(21):2540-5 1). The state of replicative senescence is considered an “activated” state by many investigators, particularly with regards to the expression of genes involved in extracellular matrix metabolism (West M D. The cellular and molecular biology of skin aging. Archives of Dermatology 1994, 130(1):87-95; Campisi J, Diniri G P, Nehlin J O, et al. Coming of age in culture. Experimental Gerontology 1996, 31(1-2):7-12).

“Nucleic acid expression vector” or “Expression cassette” refers to an assembly which is capable of directing the expression of a sequence or gene of interest. The nucleic acid expression vector includes a promoter which is operably linked to the sequences or gene(s) of interest. Other control elements may be present as well. Expression cassettes described herein may be contained within, for example, a plasmid or viral vector construct. In addition to the components of the expression cassette, the plasmid construct may also include a bacterial origin of replication, one or more selectable markers, a signal which allows the plasmid construct to exist as single-stranded DNA (e.g., a M13 origin of replication), a multiple cloning site, and a “mammalian” origin of replication (e.g., a SV40 or adenovirus origin of replication).

By “subject” is meant any member of the subphylum chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, adult, pediatric, neonatal, and embryonic individuals are intended to be covered. The system described above is intended for use in any of the above vertebrate species, since the immune systems of all of these vertebrates operate similarly.

A “coding sequence” or a sequence which “encodes” a selected polypeptide, is a nucleic acid molecule which is transcribed (in the case of DNA) and translated (in the case of mRNA) into a polypeptide in vivo when placed under the control of appropriate regulatory sequences (or “control elements”). The boundaries of the coding sequence are determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and even synthetic DNA sequences. A transcription termination sequence may be located 3′ to the coding sequence.

Typical “control elements”, include, but are not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences.

A “nucleic acid” molecule can include, but is not limited to, prokaryotic sequences, eukaryotic mRNA, cDNA from eukaryotic mRNA, genomic DNA sequences from eukaryotic (e.g., mammalian) DNA, and even synthetic DNA sequences. The term also captures sequences that include any of the known base analogs of DNA and RNA.

“Operably linked” refers to an arrangement of elements wherein the components so described are configured so as to perform their usual function. Thus, a given promoter operably linked to a coding sequence is capable of effecting the expression of the coding sequence when the proper enzymes are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

“Recombinant” as used herein to describe a nucleic acid molecule means a polynucleotide of genomic, cDNA, semisynthetic, or synthetic origin which, by virtue of its origin or manipulation: (1) is not associated with all or a portion of the polynucleotide with which it is associated in nature; and/or (2) is linked to a polynucleotide other than that to which it is linked in nature. The term “recombinant” as used with respect to a protein or polypeptide means a polypeptide produced by expression of a recombinant polynucleotide. “Recombinant host cells,” “host cells,” “cells,” “cell lines,” “cell cultures,” and other such terms denoting prokaryotic microorganisms or eukaryotic cell lines cultured as unicellular entities, are used interchangeably, and refer to cells which can be, or have been, used as recipients for recombinant vectors or other transfer DNA, and include the progeny of the original cell which has been transfected. Progeny of the parental cell which are sufficiently similar to the parent to be characterized by the relevant property, such as the presence of a nucleotide sequence encoding a desired peptide, are included in the progeny intended by this definition, and are covered by the above terms.

“Encoded by” refers to a nucleic acid sequence which codes for a polypeptide sequence, wherein the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence. Also encompassed are polypeptide sequences which are immunologically identifiable with a polypeptide encoded by the sequence.

The term “transfection” is used to refer to the uptake of foreign DNA by a cell. A cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (198 1) Gene 13:197. Such techniques can be used to introduce one or more exogenous DNA moieties into suitable host cells. The term refers to both stable and transient uptake of the genetic material, and includes uptake of peptide- or antibody -linked DNAs.

A “vector” is capable of transferring gene sequences to target cells (e.g., viral vectors, non-viral vectors, particulate carriers, and liposomes). Typically, “vector construct,” “expression vector,” and “gene transfer vector,” mean any nucleic acid construct capable of directing the expression of a gene of interest and which can transfer gene sequences to target cells. Thus, the term includes cloning and expression vehicles, as well as viral vectors.

A “selectable marker” or “reporter marker” refers to a nucleotide sequence included in a gene transfer vector that has no therapeutic activity, but rather is included to allow for simpler preparation, manufacturing, characterization or testing of the gene transfer vector.

By “pharmaceutically acceptable” or “pharmacologically acceptable” is meant a material which is not biologically or otherwise undesirable, i.e., the material may be administered to an individual in a formulation or composition without causing any undesirable biological effects or interacting in a deleterious manner with any of the components of the composition in which it is contained.

The term “angiogenesis” as used herein refers to a process whereby vessels develop from pre-existing capillaries.

By “antiangiogenic” compound it is meant a compound that inhibits angiogenesis. Such compounds may be organic or inorganic. Organic compounds include peptides and cDNAs encoding such peptides. Such compounds further include synthetic compounds, natural products, traditional medicine based and genetically engineered bioactive agents.

By “neovascularization” it is meant newly formed vessels.

### MODES OF CARRYING OUT THE INVENTION

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

A Rapid and Efficient Transgene Delivery System for Human Dermal Microvascular Endothelial Cells

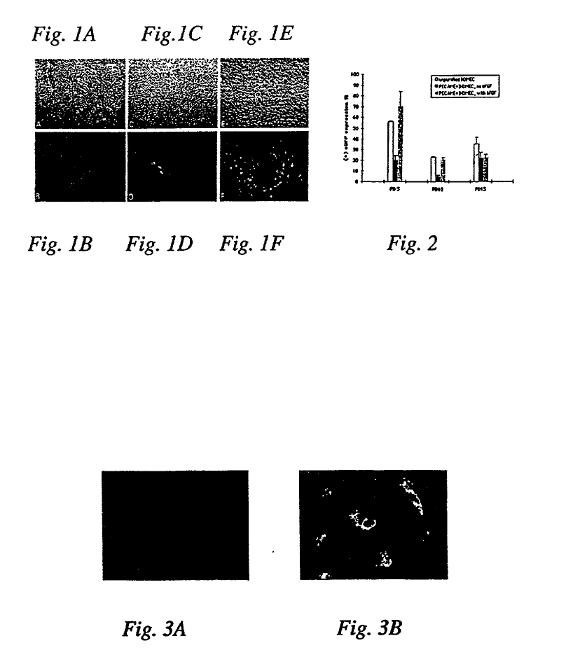

Gene transfer into vascular endothelial cells (EC) has presented particular problems. Generally low bacterial plasmid transfection efficiencies ranging between 0.1-20% are typical (Sun B, Plumpton C, Sinclair J H, et al. In vitro expression of calcitonin gene-related peptide in human endothelial cells transfected with plasmid and retroviral vectors. Neuropeptides 1994, 26(3):167-73; Tanner F C, Carr D P, Nabel G J, et al. Transfection of human endothelial cells. Cardiovascular Research 1997, 35(3):522-8; Fife K, Bower M, Cooper R G, et al. Endothelial Cell Transfection With Cationic Liposomes and Herpes Simplex Thymidine Kinase Mediated Killing. Gene Therapy 1998, 5(5):614-620). Experiments performed in support of the present invention have demonstrated that using retroviral systems in which the vector remains episomal (Nabel E G, Nabel G J. Complex models for the study of gene function in cardiovascular biology. Annual Review of Physiology 1994, 56(1):741-61) results in high efficiency gene transfer (>70%) to primary human dermal microvascular endothelial cells (HDMEC). Enhanced green fluorescent protein (eGFP; Chalfie M et al., Science, 1994, 263:802-5; Clontech Laboratories Inc., Palo Alto, Calif.) was used as a reporter when early passage, b-FGF-stimulated cells were used (Romero L et al., Journal of Cellular Physiology, 1997, 173:84-92). Photomicrographs of transfection results obtained using the LZRS-eGFP vector (Deng H et al., Nature Biotechnology, 1997, 15:1388-91; Paul Khavari, Department of Dermatology Stanford University) in HDMEC are shown in FIGS. 1A to 1F. In the figures, FIGS. 1A and 1B are primary HDMEC without retroviral infection, FIGS. 1C and 1D are primary HDMEC without treatment, and FIGS. 1E and 1F are the same cells treated for 24 hr with bFGF.

The graph shown in FIG. 2 shows an inverse correlation of reporter gene expression efficiency and passage number. Importantly, these genetically-modified HDMEC continue eGFP expression for up to four weeks in culture, display HDMEC marker expression, respond to inflammatory cytokines and 3D collagen similar to unmodified, control HDMEC.

Other genes of importance in HDMEC biology have been expressed using this expression system with high efficiency of gene transfer. One of these genes is CD34. Results of transfection of HDMEC with the LZRS-vector carrying coding sequences for the CD34 protein (Romero, et al., 1997 J. Cell Physiol 173:84-92) are presented in FIGS. 3A and 3B. Immunofluorescence staining of cells was carried out using standard procedures. FIG. 3A shows the results of primary HDMEC transfected with a sham vector carrying LacZ coding sequences. FIG. 3B shows the results of primary HDMEC transfected with the vector containing full length CD34. The cells in both cases were stained using HPCA-1 (CD34 monoclonal IgG; Monoclonal mouse anti-human CD34 (Anti-HPCA-1), (Anti-HPCL2) pure or FITC conjugated was obtained from Becton Dickinson Immunocytometry System. San Jose, Calif. Monoclonal Anti-CD34 QBEnd10 was obtained from BioGenex, San Ramon, Calif.).

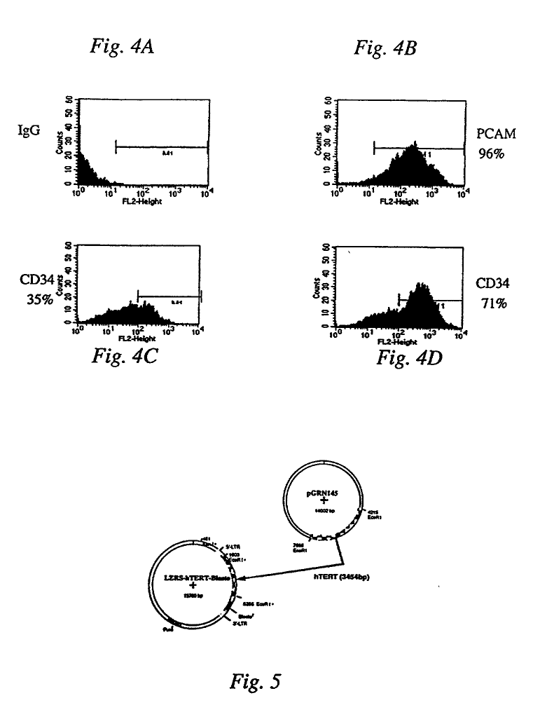

Further, fluorescence activated cell sorting was carried out on transfected cells. The results are presented in FIGS. 4A to 4D: FIG. 4A shows control IgG staining of LacZ-infected primary HDMEC; FIG. 4B shows PCAM-1 (CD3 1) positivity of LacZ-infected HDMEC, the expression of CD31 indicates that HDMEC maintained the endothelial phenotype following the expression of a transgene; FIG. 4C shows HPCA-1 staining of LacZ-infected HDMEC (a negative control); and FIG. 4C shows HPCA-1 staining of HDMEC cells transfected with and expressing full length CD34. These results demonstrate the specific expression of CD34 in cells transfected with the CD34-bearing vector. These data have been duplicated.

Taken together the data above indicate that the transgene delivery system described in Example 1 results in at least 70% expression efficiency using different recombinant cDNA constructs and thus represents an efficient and reliable method for expressing ectopic genes in primary HDMEC. The durability of the LZRS to sustain expression of hTERT in continuously dividing HDMEC in vitro is described below.

Persistent and Functionally Active Expression of Human Telomerase Reverse Transcriptase in HDMEC

A full length human telomerase reverse transcriptase (hTERT) cDNA coding sequence, in the vector pGRN145(+) (Geron Corp., Menlo Park, Calif.; Nakamura TM et al., Science, 1997, 277:955; Meyerson M et al., Cell, 1997, 90(4):785-95; Kilian A et al., Human Molecular Genetics, 1997, 6:2011) was digested with EcoRI and ligated into the vector LZRS. This vector contained a Blastocidin resistance gene useful for selection in target cells (FIG. 5). The final construct was completely sequenced to verify orientation and no mutations were detected. Expression of the hTERT transgene was driven by the MMLV LTR promoter. This plasmid was transfected into the Phoenix 293 packaging cell line (Kinsella T M and Nolan G P, Human Gene Therapy, 1996, 7:1405-13; Garry Nolan, Department of Molecular Pharmacology Stanford University) and replication-deficient high titer retrovirus was collected by standard protocols.

Early passage (population doubling 1; PD I) HDMEC were purified by affinity selection on anti-PECAM IgG-coated agarose beads according to previously published procedures (Romero, L I, Zhang, D N, Herron, G S and Karasek, M A. IL-1 Induces Major Phenotypic Changes in Human Skin Microvascular Endothelial Cells. J. Cell. Physiol. 1998; 173:84-92). The cells were then grown in Clonetics EBM defined media (Clonetics, San Diego, Calif.) and were infected with LZRS LacZ control and the LZRS-hTERT construct when the cells were at 50-60% confluency (using the method described in Example 1). The transfected cells were serially passaged without selection.

Cells were split at 1:10 into 100 mm plates and allowed to grow to confluency (−5 days); this represents -3 population doublings (PD). To test for the presence of the hTERT transgene, an RT-PCR protocol (Gibco BRL, Gaithersburg, Md.; Mullis, K. B., U.S. Pat. No. 4,683,202, issued Jul. 28, 1987; Mullis, K. B., et al., U.S. Pat. No. 4,683,195, issued Jul. 28, 1987) was designed.

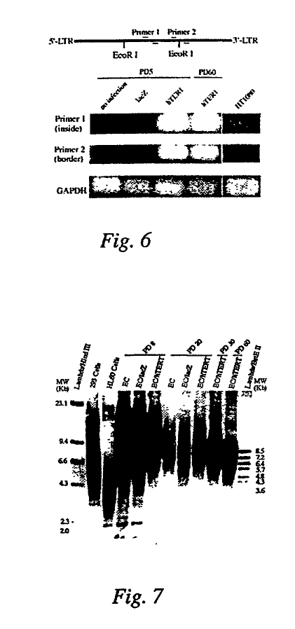

Two sets of primers were used (FIG. 6): (i) the primers in set#1 were designed to detect endogenous and transgene hTERT mRNA; and (ii) the primers in set#2 were used to amplify sequences found at the 3′ border of the LZRS-hTERT construct where the sense primer was located within the hTERT insert and the antisense primer within the vector. Total RNA was isolated from primary uninfected HDMEC, LacZ(+) HDMEC and hTERT(+) HDMEC at various PDs and RT-PCR was performed.

The results shown in FIG. 6 indicate that both transcripts were present in hTERT(+) HDMEC at early and late PDs; as expected, neither amplification product could be detected in primary uninfected or LacZ(+) HDMEC. The control cell line HT1080 (not transfected with the hTERT coding sequences) showed the amplification product associated with the endogenous telomerase but not the amplification product associated with the border/vector sequences.

To show that hTERT expression results in finctional activity two methods were used to measure telomerase activity. In the first, an ELISA-based Telomeric Repeat Amplification Protocol (TRAP; Kim, N W et al., Science, 1994, 226:2011-15) was used. In the second, the biotin-labeled PCR products from the TRAP assay were electrophoresed, blotted and detected with streptavidin-HRP (streptavidin conjugated horseradish peroxidase) chemiluminesence (Shay J D et al., Methods of Molecular Genetics, 1994, 5:263-80). In each experiment, replicate samples were heat inactivated by treatment at 65° C. for 5 minutes (hTERT-1).

These results obtained from these methods demonstrated that telomerase activity could be detected in all hTERT(+) HDMEC, whereas, both primary control and LacZ(+) HDMEC-only showed activity at early passages and lost all telomerase activity (vs hTERT-I) by PD20. Furthermore, hTERT(+) HDMEC exhibited levels of telomerase activity comparable to the immortalized 293 packaging cell line, Phoenix (Kinsella T M and Nolan G P, Human Gene Therapy, 1996, 7:1405-13). While endogenous telomerase activity could be detected by both assays in early passage primary HDMEC the endogenous hTERT mRNA could not be amplified with our RT-PCR assay (data shown in FIG. 6). These results may suggest that little endogenous transcript remains after the first few PDs in vitro whereas the hTERT subunit protein appears stably complexed with the holoenzyme that transiently persists in daughter cells.

To detect the functional consequences of telomerase activity in hTERT(+) cells, the length of telomeric DNA in the cells was examined using the TeloQuant™ assay (Pharmingen; San Diego, Calif.). Genomic DNA was isolated from a number of different cell lines when the cells were at severalpopulation densities. The DNA was digested with RsaI/HinfI followed by Southern blotting and hybridization with a biotinylated telomere probe. Positive signals were detected with streptavidin-HR-P (streptavidin conjugated horse radish peroxidase). The results are presented in FIG. 7. The mean telomere restriction fragment (TRF) length decreased from ˜8 kb to ˜6.6 kb in control cells (293 cells and HL60 cells), primary HDMEC (FIG. 7, EC) and LacZ(+) HDEMC (FIG. 7 EC/lacZ) between PD8-20. On the other hand, hTERT(+) HDMEC appeared to maintain mean TRF length at 7 kb even after PD60. Low yields of DNA isolated from senescent HDMEC cells (FIG. 7, EC and EC/lacZ) precluded determination of TRF length after ˜PD26. While not to the same degree as was observed in the HL60 negative control cells, telomere shortening was clearly found in primary HDMEC. The data presented above showed that hTERT expression in primary HDMEC resulted in persistent expression of the hTERT transgene, maintenance of telomerase activity and mean TRF length for extended periods of time in vitro.

Profound life-extension beyond the senescence point of primary HDMEC was thus observed. After PD30, both primary and LacZ(+) HDMEC showed characteristic morphologic changes of senescence (i.e., cell flattening, enlargement, and cytoplasmic vacuolization) while hTERT expressing HDMEC did not. To help visualize cellular senescence in these HDMEC populations the biomarker, acidic P galactosidase activity was used (Dimri G, Lee X, Basile G et al. A biomarker that identifies senescent human cells in culture and in aging skin in vivo. Proc. Natl. Acad. Sci. USA. 1995, 92:9363-7). Positive staining for senescent associated gal activity (SA-â-gal) was found in all cultures, but was far greater in controls than hTERT expressing cells.

Transfections of primary HDMEC were performed three times and similar results, as described above, were obtained. The initial hTERT(+) HDMEC mass cell population (designated TERT-1) has been passaged to PD123 and two others (TERT-2 and -3) have been passaged to PD45-86, respectively. These cells lines have continued to replicate normally. The phenotypic and functional characteristics of these cell lines are discussed below.

Maintenance of Endothelial Cell Phenotype in hTERT(+) HDMEC Mass Cultures

Continuously passaged hTERT(+) HDMEC maintained typical cobblestone morphology and vWF (von Willebrand Factor) expression. Microscopic examination of the cultured cells showed that HDMEC(+) hTERT cells continued to divide, exhibited normal morphology (i.e., a morphology typical of healthy, primary HDMEC) and expressed vWF (a phenotypic trait that is often lost in EC lines immortalized by other means, for example, by introducing an oncogene). The HDMEC(+)hTERT also exhibited normal contact inhibition, growth arrest upon serum withdrawal, uptake of LDL and the expression of a variety of different cell surface markers. These characteristics comprise a normal phenotype for HDMEC.

The hTERT(+) HDMEC (TERT-1) line expressed uniformly high PCAM but relatively low levels of ICAM-1 (30%), VCAM-1 (1.3%) and CD34 (19.5%) at baseline. These data show a baseline for the purpose of comparison with the data following TNFá treatment. The data indicate a similar response of hTERT(+)HDMEC cells to TNFá as normal primary cells. TNFá stimulation for 16 hr resulted in high levels of ICAM-1 on both control and TERT-1 (>99%) and comparative levels of VCAM-1 (−41%). The same treatment down-regulated CD34 expression to a greater extent in TERT-I (1.6%) vs primary cells (45%). The latter result was consistent with experiments performed in support of the present invention that studied the same characteristics of primary HDMEC CD34 expression.

To assess the differentiation and morphogenetic programming of two different TERT lines monolayer cells were exposed to 3D collagen (Collagen Biomaterials, Palo Alto, Calif.) and the cultures were examined over time using inverted phase microscopy.

Differences in tubulogenic potentials are exhibited by different TERT cell HDMEC mass cultures but both TERT-1 and TERT-3 show tubule formation. TERT-3 cells formed better tubules than ECPD5 control primary HDMEC and exhibited a more typical epitheliod morphology under basal conditions versus TERT-1 cells. The parental cells used to prepare TERT-1 were purchased from Clonetics (San Diego, Calif.) as neonatal HDMEC, whereas, the cells used to prepare the TERT-3 cell line were freshly prepared from a pool of 10 neonatal foreskins.

The above data indicated that persistent hTERT expression in HDMEC results in dramatic extension of lifespan (at least three-fold) in the absence of viral oncogene expression. This technique is a novel way to produce large numbers of primary HDMEC that retain functional characteristics. Experiments designed to extend these observations in vivo and explore potential use of these cells in gene therapy studies are presented in the Experimental section (below).

The experiments described herein have demonstrated that hTERT expression in these life-extended vascular cells (hTERT(+)HDMEC) does not affect their differentiated and functional phenotype and these cells maintain their angiogenic potential in vitro. Furthermore, hTERT(+) microvascular endothelial cells have normal karyotype and are not transformed. Compared to parental endothelial cells, hTERT expressing endothelial cells exhibited resistance to induction of apoptosis by a variety of different conditions. Such characteristics are highly desirable for designing vascular transplantation and gene therapy delivery systems in vivo.

Versatility of hTERT as General EC Life Extension Method

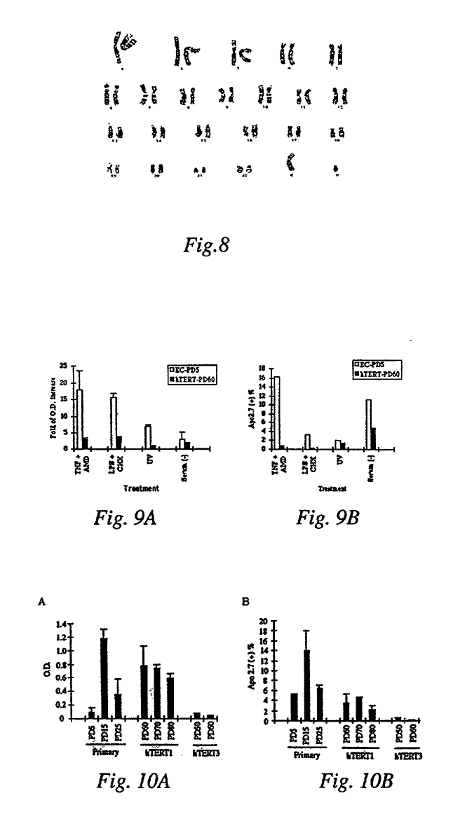

TERT-1 cells and primary parental HDMEC were submitted to the Cytogenetics Department at Stanford University (Stanford, Calif.) for chromosomal analysis. Drs. Tena Cherry and Dana Banks found that TERT-1 maintained normal 46, XY karyotype that matched parental cells in all spreads examined (20 spreads each of the TERT-1 and primary cell line). The analysis of TERT-1 cells at PD50 are shown in FIG. 8.

Other investigators have generated endothelial cells (EC) carrying hTERT, however, the methods of the present invention represent the first example of HDMEC(+)hTERT cells that are immortal and have normal karyotype. Several studies have shown genetic abnormalities in large vessel human EC (Johnson T E, Umbenhauer D R, Hill R, et al. Karyotypic and phenotypic changes during in vitro aging of human endothelial cells. Journal of Cellular Physiology 1992, 150(1): 17-27; Nichols W W, Buynak E B, Bradt C, et al. Cytogenetic evaluation of human endothelial cell cultures. Journal of Cellular Physiology 1987, 132(3):453-62) but before the disclosure of the present invention little has been known about human microvascular EC chromosomal stability. Regardless of these results, it appears that genetic abnormalities are propagated in vitro and hTERT expression does little to reverse chromosomal damage once it has occurred. The genetically and phenotypically normal hTERT(+) HDMEC mass cultures generated by the method of the present invention appears to resist chromosomal instability which allows them to display normal karyotypes, apparently indefinitely. This particular trait may or may not be related to the finding of apoptotic resistance exhibited by hTERT(+)HDMEC as described below. In addition to telomere repair, telomerase may function to repair chromosomal damage induced by a wide variety of different agents that cause DNA strand breaks (Wilke, A O, Lamb, J, Harris, P C, Finney, R D and Higgs, D R. A truncated human chromosome 16 associated with alpha thalassaemia is stabilized by addition of telomeric repeat. Nature 1990; 346:868-71; Morin, G B Recognition of a chromosome truncation site associated with alpha-thalassemia by human telomerase. Nature 1991; 353:454-6; Flint, J, Craddock, C F, Villegas, A, Bentley, D P, Williams, HJ, Galandello, R., Cao, A., Wood, W G, Ayyub, H, Higgs, D R. Healing of broken human chromosomes by the addition of telomeric repeats. Am J. Hum. Genet. 1994; 55:505-12; Hande, M P, Lansdorp, P M, Natarajan, A T. Induction of telomerase activity by in vivo X-irradiation of mouse splenocytes and its possible role in chromosomal healing. Mutat. Res. 1998; 404:205-14). In view of the teachings of the present specification and findings that apoptosis involves nuclear fragmentation and DNA breakage (Steller, H. Mechanisms and Genes of Cellular Suicide. Science 1995; 267:1445-9), telomerase activity induced by hTERT transduction may serve to repair part of the apoptosis-induced DNA damage, thereby exhibiting an “apparent” resistance to apoptosis, especially nuclear fragmentation. Alternatively, telomeres and telomere-associated proteins have been shown to influence cell fate decisions of apoptosis versus senescence (Karleseder, J, Broccoli, D, Dai, Y, Hardy, S, and de Lange, T. p53 and ATM-dependent apoptosis induced by telomeres lacking TRF2. Science 1999; 283:321-5); accordingly, hTERT may be influencing the ability of the “DNA damage sensing mechanism” to trigger the apoptotic death pathway.

The hTERT(+)HDMEC of the present invention represent the first example of immortal microvascular endothelial cells, in the absence of an activated or recombinantly introduced oncogene, having normal karyotype and normal phenotype (relative to primary microvascular endothelial cells).

Normal Growth But Altered Survival Characteristics of hTERT(+)HDMEC

Exemplary cell lines of the present invention, TERT-1, -2 and 3 HDMEC, grew to confluency at the same rates as primary HDMEC cultures, exhibited normal growth arrest at confluency and required serum for continued growth. Survival curves were generated for the TERT-1 cells using the MTT method. MTT is a water soluble tetrazolium salt yielding a yellowish solution when prepared in culture media. Dissolved MTT is converted to an insoluble purple formazan by cleavage of the tetrazolium ring by dehydrogen enzymes. Only living cells can make such a conversion due to the lack of enzyme activity in dead cells (Carmichael J. et al., Cancer Research, 1987, 47:936).

The data showed that at equal initial plating densities, TERT-1 and primary HDMEC (cell line ECPD10) survived approximately the same. However, differences in survival were evident during the first week of growth: the TERT-1 cells exhibited greater MTT reduction versus ECPD10 (2 different HDMEC pools).

Survival in growing cultures represents the net gain and loss of cells via division and death, respectively. In the MTT study no major differences were noted in the time it took to reach confluence in controls and TERT cells. Next differences in cell death rates between primary HDMEC and hTERT(+)HDMEC cells were investigated.

The ratio of stimulated nuclear apoptotic death was measured using (i) nuclear fragmentation analysis, and (ii) total percentage of cells induced to express an apoptotic-associated protein, under identical conditions for primary and hTERT bearing cell lines. Nuclear fragmentation analysis was carried out using the quantitative Cell Death ELISA assay system (Boehringer Mannheim, Indianapolis, Ind.) which detects nuclear fragmentation. Quantification of the expression of Apo2.7, an early and specific mitochondrial-associated apoptotic marker (Koester S k, Roth P, Mikulka W R, et al. Monitoring early cellular response in apoptosis is aided by the mitochondrial membrane protein-specific monoclonal antibody AP02.7. Cytometry 1997, 29(4):306-12) was carried out using FACS (to quantify on a per cell basis). The results of these experiments are presented in FIGS. 9A (cell death assay) and 9B (quantification of apoptotic marker). By both methods, TERT-1 (PD60) cells exhibit a marked resistance to apoptotic induction versus primary HDMEC (PD5).

The effect of population doubling number (PD#) on absolute baseline of (unstimulated) values of apoptosis using both nuclear and mitochondrial apoptotic analysis was determined. The results of these experiments are presented in FIGS. 10A (nuclear apoptosis) and 10B (mitochondrial apoptosis). [Protocol for 10A (and 11A): Cell death ELISA (Boehring Mannheim, Indianapolis, Ind.). Protocol for 10B (and 11B): Flow cytometry (Machine and software from Becton Dickinson, Franklin, N.J.) for Apo2.7 (Immunotech, Marseille, France).] The data in FIGS. 10A and 10B resulted from the measurement for the baseline of apoptosis.

These data suggest that midpassage primary HDMEC were particularly susceptible to apoptosis versus early passage or senescent primary HDMEC (PD25; two different pools used). The relative apoptotic resistance of senescent cells vs midpassage primary HDMEC as analyzed by both methods was consistent with previous reports in fibroblasts (Wang E. Senescent human fibroblasts resist programmed cell death, and failure to suppress bcl2 is involved. Cancer Research 1995, 55(11):2284-92; Chang E H, Jang Y J, Hao Z, et al. Restoration of the G1 checkpoint and the apoptotic pathway mediated by wild-type p53 sensitizes squamous cell carcinoma of the head and neck to radiotherapy. Archives of Otolaryngology—Head and Neck Surgery 1997, 123(5):507-12).

The TERT-1 cell line showed about the same apoptotic rates as senescent cells. The TERT-3 cell line (derived from primary EC cells as described in Example 1) showed markedly reduced apoptosis vs any of the others. TERT-1 cells do not form tubules as well as TERT-3 cells, TERT-1 cells exhibited decreased survival in 3D collagen relative to TERT-3 cells, and TERT-1 cells were derived from commercially-prepared parental cells.

The stimulated apoptosis analyses described for FIGS. 10A and 10B were repeated on cells from the same populations that had different passage numbers and used at different times. The data presented in FIGS. 11A and 11B were the results of apoptosis following a variety of inductions (ECPD25, primary controls; TERT 1PD85; and TERT-3PD60). These results proved that the hTERT(+)HDMEC cells of the present invention exhibited better survival ability.

Once again these data reproduced the previous experimental results. The data showed decreases in both nuclear and mitochondrial apoptotic events in two different TERT lines versus senescent controls under a variety of different conditions known to induce apoptosis in HDMEC (Karsan A. Tumor Necrosis Factor and Endothelial Cell Death. Elsevier Science Inc. 1998, 8(1):19-24). These observations suggest that differential regulation of EC survival factors, apoptotic inhibitors or their signaling pathways in TERT cells has occurred.

Angiostatic Factors May Be Regulated By Endogenous Matrix Metalloproteinase Activity

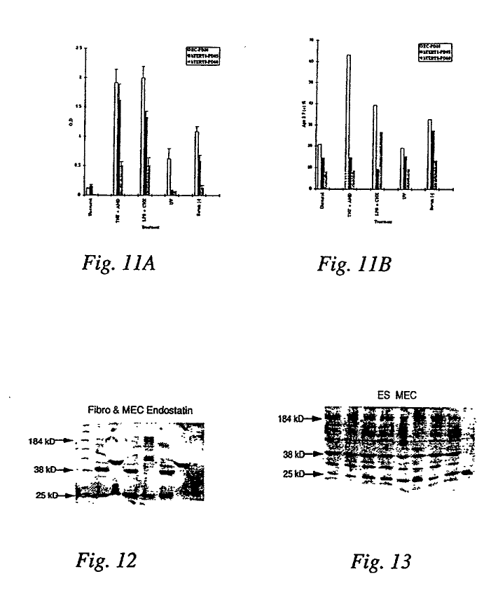

The following experiments were performed to investigate whether the microvasculopathy observed in scleroderma may relate to the presence and/or differential activity(s) of one or more endogenous angiogenic inhibitors which have been recently characterized, angiostatin (AS) and endostatin (ES) (O'Reilly M S, Holmgren L, Shing Y, et al. Angiostatin: a novel angiogenesis inhibitor that mediates the suppression of metastases by a Lewis lung carcinoma, Cell 1994, 79(2):315-28; O'Reilly MS, Boehm T, Shing Y, et al. Endostatin: an endogenous inhibitor of angiogenesis and tumor growth. Cell 1997, 88(2):277-85). A series of immunoblots using polyclonal anti-human ES antiserum (Dr. Rupert Timpl, Max Planck Institute, Martinsreid, Germany) were used to analyzed human cells, skin sections and serum for ES reactive epitopes. The results of this analysis are presented in FIG. 12 (Fibroblast and microvascular endothelial cell endostatin) and FIG. 13 (microvascular endothelial cell endostatin).

In FIG. 12, the lanes are as follows: first lane, size standards; sample lane #1=HDMEC lysate; #2=HDMEC conditioned media concentrated 100×; #3=HDMEC matrix#1 (10 mM EDTA at 37° C. for 1 hr); #4=HDMEC matrix#2 (1% Triton X100 at 4° C. for 1 hr); #5=Fibroblast lysate; #6=SFM control concentrated 100×; and #7=fetal bovine serum control.

The data in FIG. 12 showed two major ES-reactive bands in both human DMEC and fibroblast cellular lysates at approximately 25 kD and 38 kD (lanes 1, 5). The sizes of these proteins were consistent with recently published reports of ES species (Sasaki Tea. Structure, function and tissue-forms of the C-terminal globular domain of collagen XVIII containing the angiogenesis inhibitor endostatin. EMBO J. 1998, 17:4249-56). DMEC matrix prepared by EDTA extraction (matrix#1; showed the same two bands (lane 3) but matrix prepared by detergent extraction of the DMEC cultures (matrix#2) showed no 38 kD species and instead showed several higher molecular weight forms (lane 4). Lane 6 and 7 showed serum free media used to grow HDMEC (SFM) and FBS showed no ES specific bands. These data were believed to indicate that the same ES species was produced by both HDMEC and fibroblasts in the skin under these in vitro conditions. Extraction of matrix components with detergent appeared to alter the ES profile toward higher MW.

In FIG. 13, the lanes are as follows: #1=HDMEC lysate treated 16hr with 1 ug/ml TIMP-1; #2=DMEC treated 16 hr with 1 ug/ml TIMP-2; #3=untreated HDMEC control; #4=HDMEC treated 16 hr with 10 pM PMA; #5=HDMEC matrix (Triton); #6=HDMEC treated 16 hr with 50 ug/ml Concanavolin A; #7=Adult DMEC; #8=Fibroblast lysate; #9=pure ES. (TIMP-1 and TIMP-2; Tissue Inhibitor of Matrix metalloProteinases 1 and 2).

The data presented in FIG. 13 showed two important results: i) TIMPs appeared to decrease the 25kD ES species with little effect on the 38kD form in DMEC, and; ii) ConA treatment increases the 25 kD ES species with little effect on the 38 kD form in DMEC. These data were the first evidence that ES processed forms could be modulated in cultures of any cell type. Further, this was first evidence that matrix metalloproteinases may be involved in ES processing as demonstrated by modulation of ES isoforms by TIMPs. ConA is a potent inducer of MT-1 MMP and activator of pro-MMP-2 in vitro. These findings suggested that MMPs may be involved in ES proteolytic processing and further experiments will evaluate MMP patterns in different TERT-bearing cells in order to determine if, like senescent cells, an activated MMP profile may be found. If true, TERT cells may have retained the “senescent proteolytic” program and may differentially process endogenous angiostatic/matricellular factors, factors which ordinarily would not be active because senescent cells do not grow.

Mechanisms of Survival in Telomerase(+) Endothelial Cells

The data presented above suggests that constitutive, ectopic expression of telomerase in human endothelial cells in vitro can both bypass replicative senescence and achieve a state of resistance to apoptosis. Although not desiring to be bound by the particular cellular mechanisms that result in the immortal hTERT(+)HDMECs of the present invention, the following evaluation of possible cellular mechanisms may lead to a better understanding of such mechanisms. The specific cellular mechanisms resulting in the immortal hTERT(+)HDMECs are not essential to the practice of the present invention. Understanding the mechanism involved in these processes may lead to more effective therapeutic strategies for treatment of diseases involving ECs.

Evaluate Vasoprotective Factors (VEGF and NO) Expressed By hTERT(+)HDMECs

Retroviral-mediated transduction of human dermal microvascular endothelial cells (HDMEC) with the human telomerase reverse transcriptase catalytic subunit gene (hTERT) resulted in a state of apoptotic resistance that may be due to a shift in the balance between autocrine survival factors and endogenous angiogenic inhibitor expression.

Vascular endothelial cell fate decisions to enter a state of quiescence versus apoptosis are dependent on the balance of proangiogenic mediators and inhibitors of neovasculariztion via modulation of endothelial programmed cell death (Karsan A. Tumor Necrosis Factor and Endothelial Cell Death. Elsevier Science Inc. 1998, 8(1):19-24; Folkman J. Angiogenesis in cancer, vascular, rheumatoid and other disease. Nature Medicine 1995, 1:27-30; Pepper MSea. Endothelial cells transformed by polyomavirus middle T oncogene: a model for haemangiomas and other vascular tumors. 1997). This is referred to as the Angiostatic Switch. The molecular details of how some classic mitogenic factors such as vascular endothelial growth factor (VEGF) become survival factors which protect against EC apoptosis are becoming more clear (Gerber H P, McMurtrey A, Kowalski J, et al. Vascular endothelial growth factor regulates ehdothelial cell survival through the phosphatidylinositol 3′-kinase/Akt signal transduction pathway. Requirement for Flk-1/KDR activation. Journal of Biological Chemistry 1998, 273(46):30336-43). However, matricellular protein (e.g., SPARC, angiostatin, endostatin) signaling appears to attenuate mitogenic signals leading to EC growth arrest or apoptosis depending on EC activation.

Experiments performed in support of the present invention have indicated an autocrine positive-loop mechanism involving VEGF in HDMEC. VEGF expression and signaling via VEGFR1 and VEGFR2 is evaluated in hTERT(+)HDMECs of the present invention. Further, NO (Nitric Oxide) is a known vasoprotective factor involved in downstream signaling of VEGF (Ziche M, Morbidelli L, Choudhuri R, et al. Nitric Oxide Synthase Lies Downstream from Vascular Endothelial Growth factor-induced But Not Basic Fibroblast Growth Factor-induced Angiogenesis. J. Clin. Invest. 1997, 99(11):2625-2634) and experiments performed in support of the present invention indicate a correlation between downregulation of NO in HDMEC and decreased survival in vitro in scleroderma-derived HDMEC. The levels of ecNOS (endothelial constitutive nitric oxide synthase) and NOx (endothelial specific, constitutive nitric oxide synthase) is evaluated in the hTERT(+)HDMECs of the present invention and the effects of NO inhibitors on these cells is also evaluated (Example 2).

Experiments performed in support of the present invention suggest that ectopic telomerase expression in HDMEC results in i) telomeric DNA repair, ii) blockade of senescence-associated gene expression, and iii) bypass of replicative senescence via a) altered VEGF autocrine loop signaling mechanism, and b) continuous activation of both positive proliferative signals and vasoprotective/survival signals. The relative contributions of Flt-1 and KDR activation are assessed as described above and in Example 2. Furthermore, the role of NO is evaluated as described to determine if it is constitutively produced in hTERT(+)HDMECs versus a loss of cNOS and decreased NO levels in primary controls at senescence. Pharmacologic inhibition of endogenous NO shows that the survival functions of VEGF are abolished in hTERT(+)HDMECs due to blockade of VEGF signaling in downstream apoptotic inhibitor pathways.

If VEGF expression, differential regulation of VEGF receptors, or changes in NO are not observed it may be possible that other mechanisms of EC survival and resistance to apoptosis are responsible. The practice of the present invention is not limited by the particular mechanism of hTERT(+)HDMEC survival.

Measurement of Endogenous Angiostatic Factors (Thrombospondin and Endostatin)

The concept of an angiogenic switch applies equally well to embryonic development, female reproductive tissue cycling, wound repair and pathologic tissues (Hanahan D, Folkman J. Patterns and Emerging Mechanisms of the Angiogenic Switch during Tumorigenesis. Cell 1996;86(August 9):353-364). The maintenance of EC quiescence is thought to be due to the dominance of angiostatic factors over angiogenic stimulators which are present in unperturbed, normal adult tissue. Angiostatic factors include endogenous angiogenic inhibitors (angiostatin, endostatin), thrombospondin, interferons, platelet factor-4, 2 methoxyestradiol, gro-b, proliferin-related protein, matrix proteinase inhibitors and soluble cytokine receptors (Pepper M S. Manipulating angiogenesis. From basic science to the bedside. Arteriosclerosis, Thrombosis, and Vascular Biology 1997; 17(4):605-19).

However, many of these same angiostatic factors become apoptotic inducers of activated/angiogenic endothelium referred to here as the “angiostatic switch,” although the mechanisms responsible for this activity are not completely understood (Yue T L, Wang X, Louden C S, et al. 2-Methoxyestradiol, an endogenous estrogen metabolite, induces apoptosis in endothelial cells and inhibits angiogenesis: possible role for stress-activated protein kinase signaling pathway and Fas expression. Molecular Pharmacology 1997;51(6):951-62; Lucas R, Holmgren L, Garcia 1, et al. Multiple Forms of Angiostatin Induce Apoptosis in Endothelial Cells. Blood 1998;92(12):4730-4741). Because (i) many endogenous angiogenic inhibitors bind growth factors via heparin binding domains (Kupprion C, Motamed K, Sage E H. SPARC (BM-40, osteonectin) inhibits the mitogenic effect of vascular endothelial growth factor on microvascular endothelial cells. Journal of Biological Chemistry 1998;273(45):29635-40; Auerbach W, Auerbach R. Angiogenesis inhibition: a review. Pharmacology and Therapeutics 1994;63(3):265-311; O'Reilly M S, Hohngren L, Shing Y, et al. Angiostatin: a novel angiogenesis inhibitor that mediates the suppression of metastases by a Lewis lung carcinoma, Cell 1994;79(2):315-28) and (ii) growth factor withdrawal both in vitro (Hase M, Araki S, Kaji K, et al. Classification of signals for blocking apoptosis in vascular endothelial cells. Journal of Biochemistry 1994; 116(4):905-9; Levkau B, Koyama H, Raines E W, et al. Cleavage of p21Cip1/Waf1 and p27Kip1 mediates apoptosis in endothelial cells through activation of Cdk2: role of a caspase cascade. Mol Cell 1998;1(4):553-63) and in vivo (Benjamin L E, Keshet E. Conditional switching of vascular endothelial growth factor (VEGF) expression in tumors: induction of endothelial cell shedding and regression of hemangioblastoma-like vessels by VEGF withdrawal. Proceedings of the National Academy of Sciences of the United States of America 1997;94(16):8761-6) are potent EC apoptotic inducers, it is thought that these inhibitors function by inducing vascular regression of newly formed capillaries via apoptosis. Thus, an Angiostatic Switch may be defined as the induction of EC apoptosis by angiostatic factors in actively growing blood vessels. This switch mechanism may be toggled when EC cycling occurs, supporting the idea that apoptosis induction is dependent on mitotic machinery. EC quiescence is short circuited when matricellular proteins, growth factors, and intercellular junctions fail to send a survival signal. The rolelbehavior of these angiostatic factors in the hTERT(+)HDMEC of the present invention can be evaluated using methods available in the literature in view of the teachings of the present specification.

Compare Levels of Activated Matrix Metalloproteinases and TIMPs

The expression of proteolytic enzymes by sprouting endothelial cells is an early and crucial step in the angiogenic process (Pepper MSea. Endothelial cells transformed by polyomavirus middle T oncogene: a model for haemangiomas and other vascular tumors. 1997). HDMEC can express many different matrix degrading proteinases in vitro including the following: MMP-1, 2, 3 and 9; and several different serine proteinases of the plasmin-PA system (Mauch, C, Herron, G S, Bauer, E A (1999) Regulation of connective tissue turnover by metalloproteinases. In, Basic Structure and Function of the Skin. Ed., Ruth Freinkel). Proteolytic digestion of larger matrix associated precursors appears to release many types of angiogenic inhibitors and these become active as angiostatic agents. Fibronectin, angiostatin, prolactin and endostatin all are activated by proteolysis, whereas, TSP-I does not require processing. As (Angiostatin) and ES are believed to bind to and sequester growth factors via heparin-binding domains and exogenous addition of these inhibitors to proliferating endothelial cells blocks cell division and in the case of angiostatin, induces apoptosis (Lucas R, Holmgren L, Garcia 1, et al. Multiple Forms of Angiostatin Induce Apoptosis in Endothelial Cells. Blood 1998, 92(12):4730-4741). The data presented above showed that ES was produced by HDMEC and that it was differentially processed following MMP activity modulation, suggesting a role for MMPs in possibly producing biologically active forms of ES in vitro. MWs are differentially regulated during cellular aging and senescence (West M D. The cellular and molecular biology of skin aging. Archives of Dermatology 1994, 130(1):87-95). The activation profiles of MMPs and levels of TIMPs in the hTERT(+)HDMECs of the present invention are evaluated as described in Example 3, and any differences observed are then correlated with the patterns of ES processed forms. [0154] If ES and/or TSP-I expression patterns change in a reproducible and consistent manner in hTERT(+)HDMECs vs controls, these changes will be correlated with MMP activation patterns. Further, purified forms of the angiogenic inhibitors are then used to determine their effect on growth and survival in hTERT(+)HDMEC lines.

Mechanisms of Apoptotic Resistance in hTERT(+) HDMEC

It is possible that proapoptotic components of the cell cycle and endogenous apoptotic inhibitors are differentially regulated in hTERT(+) HDMEC in response to apoptosis induction. Although not desiring to be bound by the particular cellular mechanisms that result in the apoptotic resistance of the hTERT(+)HDMECs of the present invention, the following evaluation of possible cellular mechanisms may lead to a better understanding of such mechanisms. The specific cellular mechanisms resulting in the apoptotic resistance of hTERT(+)HDMECs are not essential to the practice of the present invention. The practice of the present invention is not limited by any particular mechanism of hTERT(+)HDMEC apoptotic resistance. Understanding the mechanism involved in these processes may lead to more effective therapeutic strategies for treatment of diseases involving ECs. Measurement of Cell Cycle Checkpoint Protein (pRB, p53) Expression Patterns

pRB and p53 are expressed at different levels during the cell cycle and their activities are regulated by protein phosphorylation during the cell cycle. Measurement of these expression patterns in the hTERT(+)HDMEC of the present invention can be achieved by Western/Immunoblotting with commercially available antibodies (Transduction Labs, Santa Cruz BioTech).

Measurement of CDK Inhibitor (p 16, p21, p27) Expression Patterns

Both p53 and pRB have been implicated in determining cell fate decisions involving DNA repair, cell cycle progression, arrest and apoptotic induction; however, the mechanisms for these different functions are not clear (Kasten M, Giordano A. pRb and the Cdks in apoptosis and the cell cycle. Cell Death and Differentiation 1998, Review: 132-140; Evan G, Littlewood T. A matter of life and cell death. Science 1998, 281(5381):1317-22). Likewise, induction of cellular senescence involves CDK inhibitor activity (Zhu J, Woods D, McMahon M, et al. Senescence of human fibroblasts induced by oncogenic Raf. Genes and Development 1998, 12(19):2997-3007) and changes in both p16 and p21 were found in senescent HDMEC(Watanabe Y, Lee S W, Detmar M, et al. Vascular permeability factor/vascular endothelial growth factor (VPF/VEGF) delays and induces escape from senescence in human dermal microvascular endothelial cells. Oncogene 1997, 14(17):2025-32). Phosphorylation correlates with activation functions and thus, determination of p53 and pRB hypo and hyperphosphorylation states may reveal important differences between hTERT(+)HDMECs and primary HDMEC during cell cycling, arrest and apoptotic induction. Further, CDKI levels fluctuate during checkpoints. Some of these proteins (e.g. pRB, p27 and p21) are substrates for caspase mediated degradation in EC and thus serve as valuable indicators of death pathway effector function (Levkau B, Koyama H, Raines EW, et al. Cleavage of p21Cip1/Waf1 and p27Kip1 mediates apoptosis in endothelial cells through activation of Cdk2: role of a caspase cascade. Mol Cell 1998, 1(4):553-63). The experiments described in Example 4 provide means to obtain information on the expression patterns of these proteins following apoptotic induction in hTERT(+)HDMECs.

p53 expression levels will likely increase in response to apoptotic induction and pRB, p21 and p27 will likely decrease in both hTERT(+)HDMECs and primary HDMEC. However, caspase-mediated degradation may change expression and cleavage patterns of these proteins in hTERT(+)HDMECs such that they may reveal important differences versus primary HDMEC (which are relatively sensitive to apoptotic induction). If differences are detected, these may serve as important “endpoints” for tracing effector pathways. A general caspase inhibitor (for example, ZVAD-fmk; Sigma, St. Louis, Mo.) may be used to block these differences.

These experiments will help to clarify potential upstream and downstream regulators of EC apoptotic resistance and may serve to establish a link between one type of DNA repair pathway (i.e., telomere) and programmed cell death.

Evaluation of Apoptosis Blockers (Bcl-2, Bcl-XL, A1) and the Proapoptotic Factor “Bad” and “Caspase-3”

Determination of endogenous apoptotic resistance proteins in the hTERT(+)HDMECs of the present invention directly tests the hypothesis that increased levels and/or activated forms of Bcl-2 family members are responsible for the apoptotic resistance of the hTERT(+)HDMECs. Levels of these factors are determined as follows. Bcl-2, Bcl-XL and Bad are detected by immunoblotting with anti-Bcl-2 MoAb IgG (Neomarkers) and anti-Bcl-X polyclonal IgG (Transduction Labs, Lexington, Ky.). Phosphorylated and unphosphorylated Bad can be detected as described using commercial reagents (Yano S, Tokumitsu H and Soderling T R. Calcium promotes cell survival through CaM-K kinase activation of the protein-kinase-B pathway. Nature 1998, 396:584-7). Because no A1 immunoreagents are commercially available, A1 transcript levels are determined using primers designed according to published reports (Karsan A, Yee E, Harlan J M. Endothelial cell death induced by tumor necrosis factor-alpha is inhibited by the Bcl 2 family member, A1. Journal of Biological Chemistry 1996, 271(44):27201-4). The levels of each apoptotic factor are determined before and after induction of apoptosis in hTERT(+)HDMECs and primary HDMEC controls using several different induction methods (for example, serum starvation/growth factor withdrawal and TNFalpha+AMD).

The expression patterns of one or more of these three apoptotic resistance factors will likely be increased in hTERT(+)HDMECs and Bad will likely be decreased. Further, it is possible that differential phosphorylation of Bcl-2 occurs in hTERT(+)HDMECs which contributes to their apoptotic resistance phenotype. Accordingly, if obvious changes in the absolute Bcl-2 levels of stimulated or unstimulated hTERT(+)HDMECs vs primary HDMEC are not observed, Bcl-2 phosphorylation status will be evaluated using previously described protocols (May W S, Tyler P G, Ito T, et al. Interleukin-3 and bryostatin-1 mediate hyperphosphorylation of BCL2 alpha in association with suppression of apoptosis. Journal of Biological Chemistry 1994, 269(43):26865-70).

Experiments performed in support of the present invention have demonstrated that TERT-3 cells have markedly reduced expression of caspase-3 versus primary HDMEC indicating that one possible mechanism of apoptotic resistance exhibited by hTERT(+) HDMEC is the modulation of the proteolytic death cascade via caspase-3 expression levels.

Evaluation of PI-3 Kinase/Akt Signaling Pathway Under Flow-Induced Shear Stress Conditions

There are at least two vasoprotective systems in EC which involve activation of the PI3K/Akt pathway: (i) VEGF signaling via KDR (i.e., VEGF receptor-2), and (ii) flow induced shear stress activation of PI-K/Akt. The first system represents a survival pathway, as it results in phosphorylation of Bad and release/activation of Bcl-2 to protect against growth factor mediated apoptosis in HUVEC (Gerber H P, McMurtrey A, Kowalski J, et al. Vascular endothelial growth factor regulates endothelial cell survival through the phosphatidylinositol 3′-kinase/Akt signal transduction pathway. Requirement for Flk-1/KDR activation. Journal of Biological Chemistry 1998, 273(46):30336-43). The second system also protects against apoptosis, but it is unclear whether activation of the PI3K/Akt pathway, which occurs via shear stress alone, is responsible or whether upregulated NO expression contributes to this activity (Dimmeler S, Haendeler J, Nehls M, et al. Suppression of apoptosis by nitric oxide via inhibition of interleukin-1beta-converting enzyme (ICE)-like and cysteine protease protein (CPP)-32-like proteases. Journal of Experimental Medicine 1997,185(4):601-7; Dimmeler S, Assmus B, Hermann C, et al. Fluid shear stress stimulates phosphorylation of Akt in human endothelial cells: involvement in suppression of apoptosis. Circulation Research 1998, 83(3):334-41). The activation of the PI3K/Akt pathway in hTERT(+)HDMECs will be evaluated under static vs flow conditions and compared to primary HDMEC in order to check if differences in the ability of this pathway to block apoptosis are observed.

Akt phosphorylation is assessed by immunoblotting using an antibody specific for phosphorylated Akt Ser′″ according to the manufacturer's protocol (PhosphoPlus Akt Ser 473 Kit, New England BioLabs, Beverly, Mass.). Confluent hTERT(+)HDMECs and control cells are exposed to laminar fluid flow using a cone plate viscometer at shear stress of 5-40 dynes/cm (Tsao P S, Buitrago R, Chan J R, et al. Fluid flow inhibits endothelial adhesiveness. Nitric oxide and transcriptional regulation of VCAM-1. Circulation 1996, 94(7):1682-9).

Apoptosis in hTERT(+)HDMECs and controls is induced by both TNFá+AMD and UV light before exposure to static or flow induced shear stress, followed by Akt phosphorylation analysis and detection of apoptosis (nuclear and mitochondrial). Preincubation of replicate cultures with either wortmannin (Sigma, St. Louis, Mo.), Ly294002 (Sigma) or NO inhibitor (L-NNA, 0.1-2.5 mM; Sigma, St. Louis, Mo.) serves to determine specificity of Akt-mediated apoptosis blockade in response to shear stress.

hTERT(+)HDMECs will likely show a greater activation of Akt (relative to primary cells) and lower levels of apoptosis if the effect of telomerase expression on apoptotic machinery occurs at the level of signaling through the P13K/Akt/Bad. The attenuation of the shear stress effect by Akt phosphorylation blockers and/or NOS blockers will help to determine the contribution of each of these pathways.

Development of an In Vivo Angiogenesis Model System

hTERT(+) HDMEC display a survival advantage, versus primary cells, and undergo an angiogenic response to form vascular structures in vivo. hi order to evaluate this advantage, organotypic skin equivalents containing hTERT(+) HDMEC are xenografted on SCID mice, formation of human microvasculature is measured, and its responses to proangiogenic and angiostatic factors evaluated.

Formation of Human Microvasculature

The data presented above show that hTERT(+)HDMECs retain tubulogenic pattern formation in response to matrix-derived signals, as well as, many other endothelial cell phenotypic characteristics similar to primary parental cells in vitro. However, the hTERT(+)HDMECs have two characteristics that are notable exceptions: (i) hTERT(+)HDMEC continuously divide far beyond senescence, and (ii) hTERT(+)HDMEC developed resistance to normal apoptotic-inducing agents, including matrix signals

One major obstacle to the development of in vivo angiogenesis models has been the lack of endothelial cell survival after experimental manipulation. Even with the advent of commercially-available microvascular EC and dermal equivalent systems, the seeding of endothelial cells within matrices has not allowed a reproducible tubulogenic assay to be developed. Use of the hTERT(+)HDMECs of the present invention, which display normal functional characteristics but resist apoptosis, promises to yield unique and powerful advantages over previous studies.

Dermal fibroblasts and hTERT(+)HDMECs are mixed together 1:1 and seeded into interstitial collagen gels atop polycarbonate filters which are allowed to polymerize 4-7 days in vitro. Human keratinocytes are then added to the dermal matrix and grown submerged for 4 days before stratifying by exposure to air-liquid interface for 3 days. The filters containing skin constructs are then placed as grafts onto the backs of SCID mice, allowed to heal for at least 10 days and assessed for cellular composition and immunoreactivity thereafter. Initial determinations performed on the grafts include hematoxylin and eosin (H&E) and immunofluorescence for expression of vWF, CD31, type IV collagen and laminin-I using anti-human immunoreagents. Controls include nontransduced primary HDMEC, lacZ sham transduced HDMEC, and various hTERT(+) HDMEC lines prepared according to the methods of the present invention, currently growing (PD60-100).

Graft replicate animals (4 each) are prepared for initial experiments and biopsy on the 10^{th}, 20^{th}, and 30^{th} day postgraft. After the formation of human microvessels the expression of other microvascular EC-specific surface markers (e.g. ICAM, CD32, CD34, CD36) is evaluated. Further, inflammatory cell markers (VCAM, E-selectin) are induced using intralesional injection of IL-1â and TNFá and evaluated. The expression of MMP-1, MT-1 MMP, MMP2 and TIMP2 are also assessed. Finally, expression of endostatin (ES) and TSP-1 is performed. Immunofluorescence is performed on frozen skin sections to evaluate DEJ and vascular basement membranes.

The hTERT(+)HDMEC lines of the present invention clearly display survival advantages and appear to resist environmental stresses far better than primary HDMEC. Use of these cells in skin grafts results in incorporation of the hTERT(+)HDMECs into vascular structures. In addition to the approach just described, other dermal matrices may be employed other than interstitial collagen, including: A) Matrigel (Collaborative Biochemical Products, Medford, Mass.); B) DermaGraft TC (Advanced Tissue Sciences, La Jolla, Calif.); Q AppliGraft (OrganoGenesis, Boston, Mass.) and; D) Cryopreserved de-epidermized dermis derived from cosmetic surgery tissue specimens.

Another method of making skin equivalents that specifically avoids contraction of the matrix component can be accomplished using the teachings of the present invention and the methods described by Smola, et al. (Smola, et al., 1993; Mutual Induction of Growth Factor Gene Expression by Epidermal-Dermal Cell Interaction, J. Cell Biol. 122:417-429). This report describes the incorporation of HDMEC into skin equivalent systems with survival and proliferation of these cells using the CRD (Combi Ring Dish) technology. The grafting of the CRD-silicone “bubble” transplantation chambers onto SCID mice is carried out using hTERT(+)HDMEC incorporated into the dermal matrix.

Yet another method of incorporating genetically-modified hTERT(+)HDMEC into vascular structures using the ex vivo approach is accomplished using the teachings of the present invention and the methods of Nor, et al. (Nor, J E, Christensen, J., Mooney, D., Polverini, P. 1999. Am. J. Pathol. 154:375 384). hTERT(+)HDMEC are grown in porous poly-L-lactic acid (PLA) sponges and implanted into SCID mice. A final method of developing an in vivo model to incorporate the hTERT(+)HDMEC involves implantation of a Matrigel cushion containing bFGF into the ventral subcutaneous tissue of a SCID mouse followed by intralesional injection of hTERT(+)HDMEC after 3 days. This methods utilizes the teachings of the present invention and the methods of Tony Passaniti, Ph.D. (university of Maryland, Greenebaum Cancer Center, Bressler Research Building, Baltimore).

The approach just described, i.e. creation of endothelialized dermal equivalents, could properly be termed “vasculogenesis” because EC first form vascular tubes and lumens from the clustering, realignment and remodeling of mixtures of dermal cells within the matrix. This process is clearly different from the intussusceptive formation of tubules in response to 3D collagen or Matrigel or the sprouting of vessels from pre-existing capillaries, “angiogenesis.” It is likely that the hTERT(+)HDMEC mass cell cultures of the present invention, derived from pools of human neonatal tissue, may contain a subpopulation of “de-differentiated angioblast -like” EC that could support vasculogenic growth if these cells have a survival advantage. Further, during routine purifications of HDMEC, the PECAM(−) population of cells is often discarded. This population typically represent mixtures of dermal fibroblasts, myofibroblasts, pericytes, dermal dendricytes and other uncharacterized spindle cells. These PCAM(−) cell populations may be useful in the preparation of the endothelialized dermal equivalent because they may be enriched in perivascular cells that are involved in stabilization and morphogenetic patterning of newly formed capillaries. Furthermore, perivascular cells may also be transduced with the hTERT expression vector (described in Example 1), characterized by FACS, and used to recreate a dermal environment that is far more durable than repopulation with primary cells.

Effect of fas Expression

Fas belongs to the TNFR (TNF Receptor) and signals via the death domain in its cytoplasmic tail. HDMEC do not express Fas at baseline and are not susceptible to FasL-induced apoptosis. Fas mRNA expression can be induced by incubation of normal HDMEC with plasma from patients with TTP (thrombotic thrombocytopenia purpura) and such treatment leads to HDMEC apoptosis which can be blocked by soluble anti-Fas antibody (Laurence J, Mitra D, Steiner M, et al. Plasma From Patients With Idiopathic and Human Immunodeficiency Virus-Associated Thrombotic Thrombocytopenic Purpura Induces Apoptosis in Microvascular Endothelial Cells. Blood 1996, 87(8):3245-3254; Mitra D, Jaffe E A, Weksler B, et al. Thrombotic thrombocytopenic purpura and sporadic hemolytic-uremic syndrome plasmas induce apoptosis in restricted lineages of human microvascular endothelial cells. Blood 1997, 89(4):1224-34). A recombinant Fas/anti-Fas IgG system has been employed to induce apoptosis in both human keratinocytes and dermal fibroblasts (Freiberg R A, Spencer D M, Choate K A, et al. Specific triggering of the Fas signal transduction pathway in normal human keratinocytes. Journal of Biological Chemistry 1996, 271(49):31666-9; Freiberg R A, Spencer D M, Choate K A, et al. Fas signal transduction triggers either proliferation or apoptosis in human fibroblasts. Journal of Investigative Dermatology 1997, 108(2):215-9). This system will be used to induce apoptosis in the hTERT(+)HDMECs of the present invention after incorporation into the in vivo model, described above. This system offers the advantage of triggering apoptosis without simultaneous activation of survival signals and/or DNA repair mechanisms inherent to apoptotic induction by TNFa, LPS and LTV light. Furthermore, the system is specific because anti-Fas IgG treatment induces death only in cells overexpressing Fas and is inducible, since in the absence of anti-Fas IgG, Fas does not multimerize and activate death.

hTERT(+)HDMECs are transduced with LZRS-Fas expressing the full length Fas driven by the retroviral LTR and first tested in vitro to determine if they are more or less susceptible to apoptosis versus primary controls with and without triggering death with CH-11, a Fas cross-linking antibody (Freiberg R A, Spencer D M, Choate K A, et al. Specific triggering of the Fas signal transduction pathway in normal human keratinocytes. Journal of Biological Chemistry 1996, 271(49):31666-9). Next these hTERT(+)HDMECs are incorporated into the organotypic dermal equivalent and grafted onto SCID mice. Fas expression levels are checked by IF, before and after Fas transduction, in vitro and in vivo using the same antibody. Finally, CH-11 is administered by intradermal injection near the graft site to trigger apoptosis in vivo. Apoptosis is measured in vitro and by using TUNEL staining in vivo. The in vitro method uses FACS methodology and is performed essentially as follows. Cells are treated with TNFá+actinomycin D (AMD), LPS+cycloheximide (CHX), or UV light, 16 hours prior to the experiments. Also, one group of cells is serum starved for 40 hours. The cells are incubated with primary antibody (Apo2.7 IgG, Immunotech)+PE-conjugated secondary antibody at 37° C. The data is collected and analyzed with CellQuest (Becton Dickinson) or Coulter EPICS cell sorter.

The in vitro nuclear fragmentation method is performed essentially as follows. The cells are cultured in 48-well plates and confluent for 2 days before the assay. The reagents and protocol were from Boehringer Mannheim. Briefly, the plates are centrifuged for 10 minutes at 200 g and the supernatants are removed. The cells are then lysed for 30 minutes at room temperature. The plates are re -centrifuged and one tenth of the supernatant is used for the incubation with anti-histon-biotin and anti-DNA-POD. After 2 hours incubation, the plates are washed three times and incubated with substrate solution. The absorbance is measured at 405 nm with microplate reader (BioRad).

The TUNEL assay is performed essentially as follows. The percentage of apoptotic cells are detected by the APO-BRDU terminal deoxynucleotidyl transferase (TdT)-mediated dUTP-biotin nick end-labeling assay (Gavrieli, et al., J. Cell Biol. 119: 493-501) according to manufacturer's instructions (Phoenix Flow Systems, Phoenix, AZ).

The hTERT(+)HDMECs will likely exhibit a resistance to Fas-triggered death relative to primary HDMEC in vitro but undergo apoptosis in vivo and thus serve as an inducible apoptosis model system.

In vivo Assay System for Identifying Modulators of Angiogenesis

The endostatin-HEK293 data we describe in detail below, represents an experimental concept that can be modified in a variety of ways to provide a high throughput screen (HTS) of antiangiogenic compounds for their effects on human endothelial cells by monitoring the appearance of microvascular tubular structures.

In vitro scale-up: Co-plate into “permissive” matrices (e.g. Matrigel, collagen, reconstituted basement membrane, synthetic dermal equivalents, etc.), in a microtiter well format, telomerized-fluorescently labeled human dermal microvascular endothelial cells (TMEC) together with a “bioreactor” cell type (e.g. HEK293 cells) that expresses a gene of interest. Readout would be vessel density as measured by a robotic, inverted fluorescent microscope (e.g. Gen-2, made by Universal Imaging, Inc., powered by customized MetaMorph vascular tracing software that utilizes digitizing algorithms like the MossFilter, created by W. C. Moss, as presented in detail below). Control wells representing maximum and minimum vascularization values would be included on each plate for baseline limits.

Variation of this basic method includes antiangiogenic agents including genes other than endostatin and compounds affecting vessel formation that may or may not be related to the gene being expressed. Thus, if a research program is investigating a specific gene and has a number of synthetic peptides (generated by bioinformatic molecular modeling programs) and/or immunoreagents that antagonize or mimic the effects of the gene product, the co-plating is performed in the presence of different concentrations of the compound. Examples of specific genes that could be tested include growth factors and their specific binding domains (FGF-2, E GF, VEGF 1,2,3,4, PDGF, IGF, TGF, PLGF, SF, angiopoietins, CTGF), extracellular matrix molecules and their binding domains (fibronectin, vitronectin, collagens 1, 3, 4, 8, 18, laminins 1, 5, 8, entactin, thrombospondins, fibrillins, proteoglycans), proteinase inhibitors (TIMPs, alphal macroglobulin, antiproteinases), cell adhesion molecules and their binding domains (PECAM, ICAM, VCAM, E-selectin, CD34, CD36, CD43, beta 1, 3, 5 integrins), known angiostatic genes (endostatin, angiostatin) and apoptotic inducers (TNF, fas), inflammatory mediators (interleukins, bradykinins, neuropeptides, histamines, chemokines). Compounds that can be tested include icosinoids, retinoids, vitamin D analogues, fumagillins, nitric oxides, etc.

The genetic material expressed by the bioreactor cell can derived in two ways: a) random approach; b) intelligent approach. The former approach utilizes a shotgun transfection, retroviral or other gene transduction method to express 200-500 genes in a population of bioreactor cells. The genes to be shotgun expressed in this manner may be derived from commercial sources (e.g. cDNA libraries from Clontech/BD, Stratagene, Gibco/BRL, ATCC, etc) or from custom libraries provided by the user. The type of bioreactor cell can be varied. As explained in detail below, we initially used HEK293 cells because we found that they did not form tubular structures in our Matrigel implants in vivo (thus not confounding the assay) nor form large tumors but remained as small colonies and nests of cells that expressed the transgene of interest. The system clearly can be used to test other tumor cell types to determine if gene targeting to the tumor cell of interest can affect new vessel growth, thus supporting the use of that gene for ectopic expression in vivo. For physiologic studies (e.g. wound repair) or pathologic studies not involving tumor angiogenesis (e.g. psoriasis, atherosclerosis, diabetic retinopathy, chronic ulcers) a cell type found anatomically related to the microvasculature (e.g. pericytes, smooth muscle cells, adventitial or dermal fibroblasts, dendritic cells, etc.) could be transduced with the gene of interest and co-plated or co-cultured with TGMEC and the same read-out performed.

To screen genes using the intelligent approach, companies that have already generated libraries of bioactive genes by proprietary methods (e.g. Rigel, Exelexis, Genentech, Human Genome Sciences, Millenium, AmGen, Incyte, Celomics, Hyseq, Axys, etc) may select less than 100 genes at a time to express in the bioreactor cell. Some of these companies have custom libraries that were generated by screening for their effects on endothelial cell physiologic processes (e.g. migration, cytoskeletal changes, integrin or other adhesion molecule expression, tubule formation, cytotoxicity, etc). Alternative methods of intelligent gene screening involve constructing chimeric genes containing resistance factors that allow selection pressure to be applied (e.g. hygromycin, ampicillin, etc) or inducible marker expression (tet-inducer, tamoxifen, metallothionine, etc) that will allow detection of gene of interest in the presence of the selection agent or inducer.

In vivo angiogenesis model system: Genes and compounds already screened by the above methods are then validated for their effects in vivo using SCIDS. The latter system itself can be scaled-up by implanting up to 4 grafts per mouse using surgical templates and graft harvesting techniques. This second round of screening integrates with the first by its use of the same cell types and same genes but elevates the level of functional significance to the order of preclinical selection.

In vitro multiparameter screens that map the angiogenic program: Assay systems that span specific aspects of the angiogenic cellular differentiation program, each reporting 2-3 key variables (e.g. gene expression, cell signaling, physiologic events [e.g. MMP activity, changes in cell shape, transmigration of subcellular organelles or proteins], morphometric events [e.g. cell migration, tubulogenesis, lumen formation, branching, pruning] or apoptosis, etc.) are utilized. The telomerized cell lines are required for their replicative uniformity, phenotypic expression patterns and functional characteristics. For example: A TGMEC clone is created that expresses a chimeric gene product representing a fused reporter fluorescent gene (NFP)—DNA promoter construct. A gene product (e.g. avb3 vitronectin receptor or Tie-2 Angiopoietin receptor, etc) that only is expressed during the early phase of the angiogenic program will thus monitor only this specific portion of endothelial cell differentiation. These EC lines are engineered to include key read-out indicators to monitor steps in the angiogenic/angiostatic differentiation program. An automated platform that simultaneously measures time courses and endpoints (e.g. light and fluorescence microscopy that uses microtiter plates such as the Gen-2 from Universal Imaging) could run 1-100 plates/day; HTS could screen 50-1000 compounds/day/machine and thus could be scaled-up to thousands of compounds/day (robotics required).

Ultra-HTS could be achieved by designing assays based on intensity data alone without imaging analysis. This comes after proof of principle is achieved by demonstrating that activation of specific genes, signaling pathways and subcellular events which creates the fluorescent “hit” mimics that part of the angiogenic program of interest and commits the system to an angiogenic response.

Uses and Applications of the hTERT(+)HDMEC Cells and Methods of the Present Invention

The present invention provides methods for the generation of immortal human dermal microvascular endothelial cells (HDMECs), having normal karyotype that are resistant to apoptosis. The hTERT(+)HDMEC cells are not transformed, and have no activated oncogenes (i.e., that result in malignant transformation). The cells have an essential normal phenotype as compared to primary HDMECs. These immortal cells were generated by the introduction of the human telomerase reverse transcriptase catalytic subunit gene (hTERT) into primary HDMEC. No oncogenes were used to generate the immortalized cells of the present invention. These cells have been designated hTERT(+)HDMECs.

The hTERT(+)HDMECs of the present invention have many commercial, screening, and therapeutic applications. As described above, the cells can be used to generate xenograft mice to provide an angiogenesis model useful for, e.g., screening therapeutic compounds. The cells also provides means to identify compounds that will affect expression of telomerase in HDMEC. The cells can also be used to screen compounds that facilitate or block the formation of new blood vessels.

Further, the hTERT(+)HDMECs can be used to generate new blood vessels, reline the surfaces of existing vasculature, create new vasculature and vascular structures, in subjects by injection of the cells to, for example, a site of interest. Therapeutic uses of these cells include, treatment, for example, of atherosclerosis. As blood vessels age they change how they are presented to the immune system, the hTERT(+)HDMECs of the present invention can be used to restore the vasculature and retain normal presentation to the immune system (for example, by relining arteries of the heart). The cells are also useful in methods of reversing vascular system inflammatory response.

In addition, the hTERT(+)HDMECs of the present invention provide methods of treating tumors, increasing blood flow to tumors by administering hTERT(+)HDMEC, and by increasing blood flow into tumors improve the administration of anti-tumor and/or therapeutic compounds.

Further, following the guidance of the present specification, hTERT(+)HDMEC can be created from several different human anatomic sites. In the same way, hTERT(+)EC can be created from animals, different animal anatomic sites, or from genetically-modified (e.g. transgenic) animals. The hTERT(+)EC of the present invention can be supplied as a commercial product that provides EC which are easy to grow, have a normal karyotype, display a consistent phenotype, are not transformed, and are immortal. The hTERT(+)ECs of the present invention provide the means to obtain large quantities of genetic material (e.g. for gene microarray studies) and proteins (e.g. for extracellular matrix studies).

hTERT(+)EC of the present invention can be obtained from a number of human and animal sources including, but not limited to, the following: normal neonatal foreskin, adult normal skin, and pediatric skin; as well as, adult pathologic skin derived from patients with different cutaneous disease states (including but not limited to, scleroderma, psoriasis, Epidermolysis Bullosa, hemangiomas and other vascular proliferative lesions, skin tumors, vasculitic lesions, nonhealing wounds and wounds in different stages of healing). By the methods of the present invention such cell types are established, and characterized at the molecular level (e.g., gene expression differences as determined by micro-array technology) to determine which genes are up or down regulated and whether undiscovered genes are expressed by distinct strains. Importantly, these genes and their gene products can then be tracked in the in vivo/in situ state, providing a link between various strains in vitro and their anatomic locations in the skin. This information provides investigators with details about what makes a certain vascular disease attack just one type of vessel and not another (e.g., in leukocytoclastic vasculitis) and facilitates the development of more effective and specific therapies. Creation of hTERT(+)HDMEC lines from malignant tumor-induced angiogenic vessels allows a molecular analysis of the differences between these vessels and neovascularized tissues in wounds and other skin -pathologies.

The hTERT(+)EC of the present invention also provide pharmacologic and toxicologic methods of screening and testing new drugs designed to modulate the growth of blood vessels in vivo using human EC (e.g., by incorporation of hTERT(+)HDMEC into animal models of angiogenesis and vascular remodeling). Also, hTERT(+)HDMEC derived from pathologic tissues can be incorporated into these model systems to evaluate their potential for forming new blood vessels or to influence the regression of others.

The hTERT(+)HDMEC of the present invention provide a number of in vivo therapeutic strategies, including, but not limited to, the following: 1) syngeneic/autografted hTERT(+)EC can be used as replacement cells in disease states involving inadequate or dysfunctional proliferation/regression of host EC at the site of disease via transplantation (e.g., scleroderma, keloid scars, atherosclerotic plaques, venous or arterial ulcers, diabetic vasculopathy, flap-graft sites in plastic surgery and other healing wounds with poor vascularization, etc); 2) syngeneic/autografted hTERT(+)EC can be used as gene transfer vehicles to express ectopic genes requiring vascular delivery in monogenetic diseases (hemophilia, thalasemia, cystic fibrosis, hypercholesterolemia, etc.), and autoimmune diseases (diabetes, thyroiditis, etc.); and 3) syngeneic/autografted hTERT-EC can be used as gene delivery vehicles to express ectopic genes (angiostatic factors; AS, ES, TSP, TIMPs) that would deter the proliferation and spread of occult malignant tumors during the early stages of tumor-induced angiogenesis.

Further, new evidence suggests that adult vascular tissue and/or bone marrow contains undifferentiated “white blood cells” that represent precursors to mature, differentiated EC (Asahara T, Murohara T, Sullivan A, et al., Isolation of putative progenitor endothelial cells for angiogenesis, Science 1997;275(5302):964 -7.; Shi Q, Rafli S, Wu M H, et al. Evidence for circulating bone marrow-derived endothelial cells. Blood 1998;92(2):362-7). Human adult and/or neonatal HDMEC may contain small subpopulations of these angioblastic EC precursors. Accordingly, the hTERT(+)HDMEC strains of the present invention may contain a continuously dividing, immortalized subpopulation of cells with this property and such cells can be used in the applications described above.

The experiments described herein demonstrated that microvascular endothelial cells can be effectively immortalized by hTERT alone in the absence of malignant transformation. In addition, the results described herein showed that hTERT immortalized EC exhibited functional and morphogenetic characteristics of parental cells. These hTERT(+)EC lines also display a survival advantage beyond the hurdling of replicative senescence as they appear to be more resistant to programmed cell death. Such characteristics are useful in the design of vascular model systems and therapeutic strategies for treating age-related diseases of the vasculature.

The following examples are put forth so as to provide those of ordinary skill in the art with a complete disclosure and description of how to make and use the compositions and methods of the present invention, and are not intended to limit the scope of what the inventor regards as the invention. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### EXAMPLES

### Example 1

Microvascular Endothelial Cell Preparation, Transgene Delivery System, and Transfection Method for HDMEC

Establishment of Endothelial Cell Culture

Human dermal microvascular endothelial cells (HDMEC) for making the TERT-1 cell line were purchased from Clonetics (San Diego, Calif.). Primary HDMEC (designated “lab-made”) for making TERT-2 and TERT-3 cell lines were obtained directly from neonatal foreskin samples.

Isolation and growth of primary neonatal HDMEC was performed as described by Romero, et al. (Romero, L I, Zhang, D N, Herron, G S and Karasek, M A. IL-1 Induces Major Phenotypic Changes in Human Skin Microvascular Endothelial Cells. J. Cell. Physiol. 1998; 173:84-92). Briefly, neonatal foreskin tissue obtained from Stanford University School of Medicine Labor and Delivery ward were stored less than 2 days in Hank's balanced salt solution. Tissue was sectioned into 5 mm pieces and incubated overnight at 4° C. in 50 caseolytic U/ml of dispase (Collaborative Research, Bedford, Mass.) in HBSS, pH 7.2. The epidermis was gently separated from dermis and the gentle, outward pressure was applied to release the microvasculature from dermis into the medium. The cells were centrifuged for 5 minutes at l OOOxg, plated into a 25 cm tissue culture flask pre-coated with 1% gelatin for every two foreskins and called Passage zero (Po). Growth media was EBM-2 Mv BulletKit or EBM (Clonetics).

When P₀ cells reached 80-100% confluency, they were trypsinized, mixed with anti-PECAM IgG-coated beads (Sigma, St. Louis, Mo.) in the ratio of 5-10 beads/cell and incubated for 30 minutes at room temperature. The cells bound to beads were recovered with a magnetic particle concentrator (Promega, Madison, Wis.), plated in a new gelatin-coated 25 cm flask and referred to PECAM(+) passage 1 (P1) cells. At confluency they were replated in a gelatin-coated 6-well cluster dishes for retrovirus infection.

Gene Transduction

Gene transfer was achieved by retrovirus-mediated gene transfer. A retroviral vector was used to transduce hTERT genes to the HDMEC: LZRS-hTERT. LZRS -hTert was constructed by Eco-RI digestion of pGRN145 (Geron Corp) followed by subcloning into the LZRS plasmid (Kinsella T, Nolan G. Episomal vectors rapidly and stably produce high-titer recombinant retrovirus. Hum Gene Ther 1996; 7:1405-1413). Orientation and correct sequence of the full length hTERT cDNA in LZRS was confirmed by complete DNA sequence analysis. Retroviral DNA was transfected into the Phoenix 293 arnphotropic retroviral packaging cell system, and puromycin (2.5 g/ml) was added to the culture 3 days after transfection. Packaging cells were cultured in puromycin-supplemented 1 0% fetal bovine serum medium until confluency, switched to medium without puromycin and incubated at 32° C. overnight. Retrovirus was collected into 15 ml polystyrene tubes and centrifuged at 300×g for 10 minutes to remove contaminated cells before they were stored at −80° C.

Viral titers determined by the infection of NIH-3T3 cells were estimated at approximately 5×10⁶/ml. Retroviral infection of HDMEC was performed as follows. On infection day, cells were incubated briefly in growth medium supplemented with 5 g/ml polybrene for 10 minutes. The medium was then replaced with 2.5 ml of polybrene supplemented retroviral supernatant. Plates were centrifuged at 300×g at 32° C. for 1 hour followed by incubation at 32° C. for 5 additional hours.

The expression of hTERT in the retroviral vector was driven by Moline murine leukemia virus 5′ long terminal repeat (5′-LTR) promoter. Telomeric Repeat Amlification Protocol (TRAP) Assay [0204] Either a standard protocol (Kim and Wu, Nucleic Acids Research 25(13):2505-2597(1997)) or a PCR-ELISA based protocol (Boehringer Mannheim) was used to measure the telomerase activity from the hTERT transgene. Typically, the PCR-ELISA method was for HDMEC. For visualizing the DNA ladder with the standard protocol, 1000 or 5000 cell equivalents were analyzed. For PCR-ELISA assay, 2000 cell equivalents were used. The PCR-ELISA protocol was provided by the assay kit manufacturer (Boehringer Mannheim).

A quantitative PCR-ELISA TRAP assay showed that telomerase activity of hTERT(+) HDMEC was maintained for over 100 PDs and the level of telomerase activity achieved in hTERT(+)HDMEC was comparable to that expressed by the 293 human embryonic kidney tumor cell line. By contrast, parental HDMEC expressed endogenous telomerase transiently at early PD. Using RT-PCR, the presence of hTERT RNA transcribed from the transduced retroviral vector at PD60 in HDMEC was also confirmed.

Further, different parental EC strains were shown to senesce at different PD, exhibiting typical flattened cell morphology and senescence-associated (SA) beta -galactosidase activity between PD 19-60. By contrast, none of the hTERT(+)HDMEC lines showed significant SA-beta gal activity and morphologically appeared similar to early passage, proliferating primary EC. Thus, the ectopic expression of hTERT in EC extended the replicative lifespan of all EC strains examined to over twice that of primary EC, technically defming these TERT -EC lines as immortalized (Shay, J W, Wright, W E, Werbin, H., Defining the molecular mechanisms of human cell immortalization. Biochem. Biophys. Acta 1991; 1072:1-7).

Telomere Length Assay

Isolation of genomic DNA and development of mean TRF (telomere restriction fragment) Southerns were performed according to published procedures (Harley, et al., Nature 345: 458, 1990; Allsopp, R P, et al., PNAS 89: 10114, 1992; and Vaziri et al., Am J Hum Genet. 52:661, 1993). Briefly, the genomic DNA (3 ig) was digested with HinfI/Rsa I and run on 0.6% agarose gel. The gel was transferred onto a positively charged nylon membrane, which was hybridized at 65° C. overnight. Signal on the membrane was detected by chemiluminescence. Calculation of mean TRF length followed a standardized procedure (Levy, M. et al. J. Mol Biol. 225:95 1, 1992).

To examine the effects of hTERT expression on EC telomeres, telomere lengths were assessed by telomere restriction fragment (TRF) Southern analysis. The change in telomere length was evaluated for several representative hTERT(+)HDMEC clones as a function of PD. Telomere shortening was observed for these clones up to PD 100-120 followed by consistent stabilization at approximately 4.5-5 kbp by PD 100-120.

RT-PCR for Telomerase Transcripts

Primers for RT-PCR were as follows:

in hTERT gene—sense: CACCTCACCCACGCGAAAA; and

anti-sense: CCAAAGAGTTTGCGACGCATGTT;

at the border of hTERT and retroviral LZRS sequence—sense: TCCTGAAAGCCAAGAACGCA; and

anti-sense: GACCAACTGGTAATGGTAGCGA.

The sample RNA was isolated by using TRIZOL (Gibco). The RT-PCR was performed by a one-step RT-PCR system (Gibco).

Flow Cytometry

The antibodies used for flow Cytometry were anti-PECAM (Becton Dickinson), ICAM (Pharmingen), and Apo2.7 (Immunotech). Some cells for measuring ICAM were stimulated with TNFá (100 ng/ml) for 15 hours before the assay. For apo 2.7 assay, the cells were treated with TNFá+actinomycin D (AMD), LPS+cycloheximide (CHX), or UV light 16 hours prior to the experiments. One group of cells were serum starved for 40 hours. The cells are incubated with either PE conjugated primary antibody or un-conjugated primary antibody+PE conjugated secondary antibody at 37° C. The data was collected and analyzed with CellQuest (Becton Dickinson) or Coulter EPICS cell sorter.

Parental HDMEC and hTERT(+)HDMEC lines showed high PECAM-1 reactivity (FIG. 4B). Expression of von Willebrand factor and LDL uptake also showed no differences between parental and hTERT(+)HDMEC lines. Basal and TNFá-stimulated cell surface expression of ICAM-1, VCAM-1 and E-selectin were similar in both parental and hTERT(+)HDMEC lines. The data showed that hTERT(+)HDMEC lines continuously passaged in vitro over twice the normal replicative lifespan of primary EC exhibit both the functional and differentiated phenotype of early passage, primary EC.

Tubule Formation on Matrigel or Following 3d Collagen Overlay

Tubule formation on Matrigel. Matrigel (Collaborative Biomedical Products) was placed on ice and allowed to thaw overnight in a darkened cold room or refrigerator. Three hundred fifty microliters of the Matrigel was layered onto a pre-chilled well of a 6-well plate and then placed in an incubator at 37° C. for one half hour for the Matrigel to solidify. About 350,000 cells in M199/15% FBS/10 U/ml heparin/16 ig/ml ECGF were then seeded onto the matrix and allowed to incubate at 37° C., 5% CO2 environment.

Collagen Overlay. Primary HDMEC or hTERT(+)HDMEC were overlaid with a 1: 1 mixture of Vitrogen 100 (Celtrix) and 2×Iscoves (Gibco). Adding a small amount of NaOH to the mixture brought the color back to the original of 2×Iscoves. Solidification of the collagen gel occurred within 30 min., followed by incubation at 37° C. Plates were photographed at 8 and 24 hour using the Zeiss inverted microscope.

Morphogenetic responses were evaluated by exposing parental HDMEC and hTERT(+)HDMEC (TERT-1, TERT-2, and TERT-3) cell lines to 3D type I collagen. Both early passage parental and hTERT-bearing HDMEC cell populations (at all passage numbers tested) responded similarly by efficiently forming “angiogenic webs;” late passage, senescent HDMEC did not form such webs. Further, senescent HDMEC did not form tubules in 3D collagen. The TERT-1 cell line did not form tubules well in 3D collagen. Similar responses were seen for the cell lines when tested for their response to Matrigel. A commercial source of EC was used to prepare TERT-1, whereas, TERT-2 and TERT-3 were derived from a pool of freshly obtained primary neonatal EC.

Cell Death ELISA Assay

Cells to be tested (including primary HDMEC and hTERT(+)HDMEC lines) were cultured in 48-well plates and maintained at confluency for 2 days before the assay. The reagents and protocol, Cell Death ELISA Assay kit, were from Boehringer Mannheim. Briefly, the plates were centrifuged for 10 minutes at 200 g and the supernatants were removed. The cells were then lysed for 30 minutes at room temperature. The plates were re-centrifuged and one tenth of the supernatant was used for the incubation with anti -histon-biotin and anti-DNA-POD. After 2 hours of incubation, the plates were washed three times and incubated with substrate solution. The absorbance was measured at 405 nm with microplate reader (BioRad).

The basal apoptotic rate was monitored in HDMEC and it was found that both early and late passage parental HDMEC showed lower nuclear fragmentation relative to mid passage HDMEC with differences reaching statistical significance for PD 15 vs both PD5 and PD25. The effect of PD on apoptosis in primary HDMEC cultures was verified by FACS analysis of Apo 2.7 expression, an apoptotic-specific mitochondrial protein. Two different hTERT(+)HDMEC cell lines, TERT-1 (PD 60, 70, 80) and TERT-3 (PD50, 80), showed results comparable to early and late passage parental HDMEC.

Apoptosis was also evaluated after stimulation with several different EC apoptotic inducers using two hTERT(+)HDMEC lines (TERT-1 and TERT-3) and late passage, presenescent parental HDMEC as controls. Four different conditions for inducing EC apoptosis all showed the same result that hTERT(+)HDMEC resisted apoptotic induction relative to primary HDMEC. Except for TNFá+AMD induction in TERT-1 cells, both hTERT(+)HDMEC lines expressed statistically significant lower nuclear fragmentation versus controls in response to all treatments. LPS+CHX induction showed significantly decreased Apo2.7 expression in TERT-1 vs control, whereas, other treatments did not reach statistical significance. The TERT-3 line that exhibited lower baseline apoptosis generally showed the lowest stimulated apoptotic rates. UV light-induced nuclear fragmentation and Apo2.7 expression appeared to reveal the most dramatic differences between primary and both TERT-1 and -3 lines.

Growth Patterns and Karyotype Analysis

The growth patterns of hTERT-EC lines were compared and no significant differences in their growth rates compared to parental EC were seen. hTERT(+)HDMEC lines showed contact inhibition and exhibited normal pRB phosphorylation patterns in response to serum deprivation and hydroxyurea-induced cell cycle arrest. Furthermore, none of the hTERT(+)HDMEC lines formed colonies in soft agar.

Following mitotic arrest with Colcemid®, monolayer cell culture in log phase growth were harvested by standard cytogenetic methods of trypsin dispersal, hypotonic shock with 0.075 M KCl, and fixation with 3:1 methanol/acetic acid fixative (Barch, M. J., T. Knutsen, et al., Eds. (1997). The AGT cytogenetics laboratory manual. New York, Lippincott-Raven). Mitotic cells slide preparations were analyzed by the GTW banding method (Seabright, M. (1971). “A rapid banding technique for human chromosomes.” Lancet 2: 971-972).

G-banding and cytogenetic analyses showed parental HDMEC have a normal diploid karyotype which was maintained upon immortalization by introduction of hTERT. Taken together, these results indicate that introduction of telomerase into normal human EC does not lead to abnormal growth patterns, cell transformation, or genomic instability.

The results presented above show, the general applicability of using ectopic expression of hTERT to bypass replicative senescence while maintaining EC phenotypic and morphogenetic characteristics in vitro. Upon stable transfection or retroviral transduction of hTERT, telomerase activity was detectable in all EC and telomere lengths decreased with time in culture and then stabilized. To date, hTERT(+)HDMEC lines of the present invention, both clones and mass cultures, have achieved PI)s (PD60-130) over twice that of parental or control vector transduced cells (PD30-50) and therefore are considered “immortal” (Shay, JW, Wright, WE, Werbin, H. Defining the molecular mechanisms of human cell immortalization. Biochem. Biophys. Acta. 1991; 1072:1-7). The hTERT(+)HDMEC lines of the present invention have been continuously passaged without evidence of altered morphology or changes in growth patterns.

### Example 2

Evaluation of Expression of Vasoprotective Factors (VEGF and NO)

VEGF Analysis

VEGF transcript and protein expression are analyzed according to the following methods including ELISA and semiquantitative RT-PCR. A number of different hTERT containing EC cell lines, including the cell lines described herein, are screened at several population doubling points (e.g., PD30, 60 and 90, versus vessel-matched primary EC cultures at senescence. First, the cell lines are evaluated using the ELISA assay. If major increases or decreases in VEGF concentrations are observed, transcript analysis is performed via semiquantitative RT-PCR on representative cell lines. In the event of the observation of consistent patterns, representative cell lines are compared to primary EC at early and mid PD.

RNA Isolation

Total RNA is isolated from cultured HDMEC and hTERT(+)HDMEC using the Trizol method (Gibco BRL), according to the manufacturer's procedure and then stored at −80° C. until use.

RT-PCR and Semi-Quantitative PCR

The primers for PCR are as follows:

GAPDH sense
(5′-AATCCCATCACCATCTTCCA-3′), and
antisense
(5′-GTCATCATATTTGGCAGGTT-3′) oligonucleotides;
VEGF sense
(5′-CCATGAACTTTCTGCTGTCTT-3′), and
antisense
(5′-ATCGCATCAGGGGCACACAG-3′), oligonucleotides.

The amplification products are predicted to be 558 bp for GAPDH, and 249 bp for VEGF. The VEGF primers are chosen in exon I and exon 3 of the VEGF gene resulting in a PCR product of 294 bp irrespective of the splice-form produced. RT-PCR is carried out using 5 ug of total RNA extracted from cultured endothelial cells. After denaturation in diethylpyrocarbonate-treated water for 10 minutes at 70° C., RNA is reverse-transcribed into first strand cDNA using SuperScriptII Rnase H-reverse transcriptase (10 units/reaction, Gibco BRL) and 0.5 ig of oligo (H) as primer, at 42° C. for 50 min in a total volume of 20 μl in a buffer containing (20 mM Tris-HCl, pH 8.4, 50 mM KCl, 1.5 mM MgCl₂ 1 mM dNTP, 10 mM dithiothreitol, 20 units Rnasin). Reverse transcriptase is inactivated at 70° C. for 15 min and the RNA template was digested by Rnase H at 37° C. for 20 min. Each experiment includes samples devoid of reverse transcriptase (negative controls) to exclude amplification from contaminating genomic DNA.

Semi-quantitative RT-PCR amplification is performed with a PTC 225 thermal cycler (MJ Research), following a 1 minute period of denaturation at 94° C., under the following conditions: denaturation at 94° C. for 30 seconds, annealing at 55° C. for 30 seconds, and extension at 72° C. for 30 seconds, for a total of 30 cycles. The assay mixture contained 20 mM Tris HCl, pH 8.4, 50 mM KCl, 1.5 MM MgCl₂ 0.1 μM of oligonucleotide primers, dNTPs (100 iM of dATP, dGTP, dTTP, 10 iM dCTP), 0.5 μCi of[³²P] dCTP, 0.5 units of Taq DNA polymerase, and 5 il of a hundred fold diluted cDNA mixture. The final product is extended for 3 min at 72° C. In each experiment, RT positive controls (templates containing cDNA encoding for VEGF) and negative control (without DNA) are included.

The PCR products are then subjected to electrophoresis on 6% (w/v) acrylamide gels. Radioactivity in each band is quantified by the storage phosphorimaging technique. The screens are scanned using a Fuji BAS 2000. The signal is quantified in Photo Stimulating Luminescence (PSL) units using the Tina image analysis software. Results are expressed for each sample as band intensity relative to that of GAPDH. An optimum number of PCR cycles is determined in the region of exponential amplification. Logarithmic dilutions of the cDNA mixture are used to verify the linear correlation between the intensity of the radioactive signal and the initial amount of cDNA.

VEGF Elisa

96-well plates coated with anti-human VEGF monoclonal antibody are purchased from R&D Systems (Minneapolis). HDMEC or hTERT(+)HDMEC culture supernatants are added into the wells and VEGF is bound by the immobilized antibody. After extensive washing, a peroxidase linked polyclonal antibody specific for VEGF is added to the wells; after washing, a peroxidase substrate solution is added and the plates were incubated for 5 minutes at room temperature. Optical density is measured at 620 nm with an ELISA plate reader (BioRad).

VEGF Receptor Analysis

VEGF receptors, Flt-1/VEGFRI and flk/KDR/VEGFR2 are analyzed by immunoprecipitation with anti-human Flt-1 and anti-KDR IgG (Santa Cruz BioTech, Santa Cruz, Calif.) according to standard procedures (Herron G S, Banda M J, Clark E J, et al. Secretion of metalloproteinase by stimulated capillary endothelial cells. II. Expression of collagenase and stromelysin activities is regulated by endogenous inhibitors. J. Biol. Chem 1986, 261:2814-2818). Phosphorylation of each receptor is assessed by immunoprecipitation followed by immunoblotting with murine anti-human phosphotyrosine IgG (Upstate BioTech, New York, N.Y.) according to the protocol described by (Kupprion C, Motamed K, Sage EH. SPARC (BM-40, osteonectin) inhibits the mitogenic effect of vascular endothelial growth factor on microvascular endothelial cells. Journal of Biological Chemistry 1998, 273(45):29635-40). Data are normalized to total protein and blots are reprobed with beta actin (1:2000 in TBS/0.1 % Tween 20/3% BSA for 2 hrs.; Boehringer Mannheim, Indianapolis, Ind.). Experiments are typically performed in duplicate on PD5 and PD25 primary HDMEC and PD30 and PD60-90 hTERT(+)HDMECs in defined growth media (EBM; Clonetics, San Diego, Calif.) at 90% confluency in the presence of 0.1-1.0 nM VEGF (Pepro Tech).

NO Analysis

ecNOS transcripts, protein levels and concentration of total nitrogen oxides (NOx) are determined by semi-quantitative RT-PCR, western blotting and chemiluminescence according to standard protocols essentially as described below. hTERT(+)HDMECs at PD30, 60 and 90 are compared to PD5 and PD25 of primary control EC at subconfluency (50-80%).

SDS-PAGE is performed using 8% separating gel according to previously published procedures (Chan, V T, Hultquist, K, Zhang, D N, Romero, L I, Lao, D and Herron, G S. Membrane type matrix metalloproteinase expression in human dermal microvascular endothelial cells. J. Invest Dermatol. 1998; 111: 1153-1159 ). 80-90% confluent HDMEC or hTERT(+)HDMEC at third passage (48 hr after plating) are washed in cold PBS pH 7.4, and solubilized in 1 % SDS, 10 mM Tris pH 7.4. Cell lysates are boiled for 5 minutes and centrifuged at 2500×g for additional 5 minutes to remove insoluble material. Protein concentration is determined using the Bradford assay (BioRad). An equal amount of protein (17.5 μg) is loaded into each lane, separated by SDS-PAGE, and transferred to nitrocellulose by electroblotting at 4° C. The nitrocellulose membrane is blocked in a solution containing 1% BSA, 10 MM Tris pH 7.5, 100 mM NaCl, 0.1% Tween 20 at 4° C. overnight. After 3 washings of 10 min with PBS/1 % Tween 20 the membrane is incubated with 1:250 dilution of the anti-human endothelial eNOS IgG in the blocking solution for 4 h at room temperature. Duplicate samples are also reacted with the anti-human smooth muscle actin IgG as controls. After decanting the primary antibody and washing as above, the membrane is incubated with a horseradish peroxidase labeled-sheep anti-mouse antibody for I hour at room temperature. The membrane is developed using the luminescent method of ECL (Amersham) after exposure to substrate for 5 minutes followed by visualization on X-ray film. The film is then photographed and digitally analyzed using the Electrophoresis Documentation and Analysis System 120 by Kodak according to the manufacture's instructions.

RT-PCR Analysis for eNOS

Reverse transcriptase polymerase chain reaction (RT-PCR) is used to assess eNOS mRNA expression by 80-90% confluent, normal, SSc, HDMEC, hTERT(+)HDMEC (at various passage numbers) and Loc Scl DMEC approximately 48 hr after plating (2^{nd} to 3^{rd} passage). 10 ng total RNA, isolated with STAT-60 (Tel Test “B” Inc., Friendswood, Tex.) is used as template for cDNA synthesis in a volume of 50 il according to manufacture's recommendations (Invitrogen, San Diego, Calif.). For PCR amplification, 3 il of cDNA is used as a template and amplification conditions are 95° C. for 5 minutes followed by 95° C. for 1 minute, 58° C. for 1 minute and 72° C. for 1 minute for 30 cycles in a Perkin Elmer Cetus 9600 thermal cycler. Amplification is performed in a total volume of 50 μl containing 1.5 mM MgCl₂, 0.1 mM of each nucleotide, 5 μmol of each primer, and 2.5 U of Taq Polymerase (Perkin Elmer, Branchburg, N.J.). To amplify the 400 bp eNOS cDNA fragment the upstream primer used is 5′GTG ATG GCG AAG CGA GTG AA 3′ and the downstream primer is 5′CCG AGC CCG AAC ACA CAG AAC 3′. Replicate samples are used to amplify the 300 bp GAPDH cDNA using upstream primer 5′GGG GAG CGA GAT CCC TCC AAA ATC AAG TGG GG and downstream primer 5′GGG TCA TGA GTC CTT CCA CGA TAC CAA AGT TG. The PCR products (10 il) are analyzed on 1.5% agarose gel electrophoresis, stained with ethidium bromide, destained and photographed under ultraviolet light using the Electrophoresis Documentation and Analysis System 120 by Kodak according to the manufacture's instructions. Relative eNOS transcript levels are determined by comparing the ratio of eNOS:GAPDH densitometric units for HDMEC, hTERT(+)HDMEC (at various passage numbers) SSc, Loc Scl and control samples. Significant differences between samples are deter-mined by ANOVA using the Statview SE+program (Abacus Concepts Inc.). p<0.05 was considered statistically significant.

Measurement of Cell Secreted Nitrogen Oxides

Second passage, PECAM(+) DMEC from, 3 adult control, 3 SSc, HDMEC, and hTERT(+)HDMEC and 3 Loc Scl patients are plated in duplicate at 50% confluency on gelatin-coated 35 mM petri dishes. At confluency (5×10′ cells) Complete Media is removed and plain DMEM is substituted. One dish per sample is stimulated with 1×10⁷ M calcium ionophore A23187 (Sigma) in plain DMEM. All media is collected at 16 hr, snap frozen and stored at −20° C. for measurement of nitrogen oxides (NOx).

NOx in cell media is measured using a chemiluminescence apparatus (model 2108, Dasibi Corp., Glendale, Calif.) as previously described (Tsao P S, McEvoy L M, Drexler H, Butcher E C, Cooke J P; Enhanced endothelial adhesiveness in hypercholesterolemia is attenuated by L-arginine, Circulation 1994 May;89(5):2176 82). An aliquot (50 μl) of media is injected into boiling acidic vanadium (111) chloride. This technique utilizes acidic vanadium (III) chloride at 98° C. to reduce both N02- and N03- to NO, which is detected by the chemiluminescence apparatus after reacting with ozone. Signals from the detector are analyzed by computerized integration of curve areas. Standard curves for NaNO2/NaNO3 are linear over the range of 50 pM to 10 riM. NOx values are analyzed using the Anova Statview SE+program as described above; p<.0.05 was considered statistically significant.

Pharmacologic Blockade of cNOS

To test the sensitivity of hTERT(+)HDMECs versus primary control HDMEC to NO inhibitors, cells are treated with a potent competitive inhibitor of cNOS, N-w-nitro-L-arginine (L-NNA) (Sigma, St. Louis, Mo.) at 0.1-2.5mM concentrations. Survival curves (MTT; Sigma, St. Louis, Mo.) are measured for the cell lines following L-NNA treatments at baseline and in the presence of apoptotic inducers (TNFâ/AMD, LPS/CHX, UVC, SFM; Sigma, St. Louis, Mo.) and recombinant Endostatin. The survival curves are compared among hTERT(+)HDMECs and controls.

### Example 3

Comparison of Levels of Activated Matrix Metalloproteinases and TIMPs

Evaluation of TSP-1

Experiments performed in support of the present invention have shown that TSP-1 was differentially expressed in HDMEC derived from pathologic skin samples (e.g. Junctional Epidermolysis Bullosa; JEB) vs neonatal HDMEC Immunofluorescence micrographs showed TSP-1 reactivity as wispy confluent deposits in control cell matrix, whereas JEB HDMEC retained very little cytoplasmic TSP-1 and no deposition into the matrix (photo-exposure time control TSP=14 sec; photo-exposure for JEB TSP=38 sec). Occasional JEB HDMEC stained weakly for TSP-1. TSP-1 levels are evaluated in the hTERT(+)HDMECs of the present invention versus primary controls using a combination of IF microscopy and RT PCR.

Evaluation of ES

ES isoforms produced are evaluated for several hTERT-bearing cell lines compared to primary HDMEC controls at different PD using the same immunoblotting procedures described above for evaluation of ES.

Measurement of MMP Activities

Replicate hTERT(+)HDMECs (PD30, 60, 90) and primary controls (PD5, PD25) are seeded at equal densities, grown to confluence and switched to EBM media (Clonetics, San Diego, Calif.) containing growth factors, but no serum, for 72 hrs. Media is collected and total protein measured (Pierce, Rockford, Ill.). Zymography is performed according to previously published procedures (Herron G S, Banda M J, Clark E J, et al. Secretion of metalloproteinase by stimulated capillary endothelial cells. II. Expression of collagenase and stromelysin activities is regulated by endogenous inhibitors. J. Biol. Chem 1986, 261:2814-2818; Chan, V T, Hultquist, K, Zhang, D N, Romero, L I, Lao, D and Herron, G S. Membrane type matrix metalloproteinase expression in human dermal microvascular endothelial cells. J. Invest Dermatol. 1998, 111:1153-1159). In addition, conditioned media are analyzed by a fluorescent substrate assay (FSA).

The FSA is an assay system for MMP activities based on recently-developed fluorogenic substrates that utilize 7-methoxycoumarin (MOC)-labeled MMP-specific small peptides (“Knight” substrate; Knight CG. Fluorimetric Assays of Proteolytic Enzymes. Met Enzymol 1995, 248:18-34). Release of MOC from the 2,4-dinitrophenyl-quenched peptide by active MMPs results in proportional increases in fluorescence with time. Activities of recombinant MMP-2 and MMP-9 using the Knight substrate were readily detectable and yielded linear initial reaction rates over 10 minutes. Calculated initial rates of hydrolysis of the Knight substrate (4 uM) by MMP-2 (1.2 nM), MMP-9 (1.2 nM), and MMP-2 (0.6 uM) plus MMP-9 (0.6 uM) were 571.1 units/min, 208.2 units/min, and 242 units/min, respectively.

Activity measurements of conditioned media (CM) from primary normal human (NHK) and immortalized keratinocyte (NIK) cultures showed low net MMP activity detectable only in PMA-treated NHK and only after APMA activation (10.1 units/min/ug total protein). Values for concentrated CM from HDMEC cultures+/PMA considerably higher and exhibited a linear increase with increasing volume of sample. Activities were completely blocked by 1, 10 phenanthraline and partially blocked by rTIMP-1. Reverse zymography confirmed the presence of TIMPs.

The FSA is a fast and reproducible method for quantifying net MMP activities in CM. TIMPs decrease the sensitivity of this assay system. Accordingly, the assay system is used in combination with zymography and/or after removal of TIMPs with anti-TIMP affinity beads.

### Example 4

Measurement of CDK Inhibitor Patterns

Cellular extracts are prepared, total protein measured (BCA, Pierce) and irmunoblotting performed using anti-p53, p21 (Oncogene Research, Cambridge, Mass.), pRB, p 16, p27 (Santa Cruz Biotechnology, Santa Cruz, Calif.) according to published protocols.

Confluent hTERT(+)HDMECs and late passage primary HDMEC are induced to undergo apoptosis by two different methods, serum starvation/growth factor withdrawal and TNFá+AMD (actinomycin D). Time course studies are performed to assess changes in expression patterns at early and late apoptotic induction time points. Further, the effects of exogenous VEGF stimulation on phosphorylation status of these cell cycle proteins will be evaluated.

### Example 5

Superior Durability of Telomerase Expressed Human Dermal Microvascular Endothelial Cells

Advantages of the Present Invention

Uniform Cell Populations

Herein the present invention demonstrates that hTERT(+)HDMEC retain the phenotypic and functional characteristics of young primary cells 3-5 times longer than primary cells (Yang et al 1999 J. Biol. Chem. 274:37:26141-48). These cells can be greatly expanded in culture thus providing the first ever uniform mass cell culture for angiogenic assay systems. Because all cells in the culture were derived from the same tissue source, they reproducibly respond to angiogenic and angiostatic stimuli the same way from generation to generation. All cells that are not life-extended via hTERT expression are selected against by continuous passaging beyond senescence of primary cells and thus cannot variably affect cell-cell interactions or present differential responses to stimuli. The hTERT(+)HDMEC mass cultures can either be used as mixed populations or can be fractionated further into different subpopulations representing different anatomic locations within the original tissue (e.g. precapillary arterioles vs postcapillary venules) via FAC sorting (FIG. 4). This provides a pure, uniform and immortal cell population for incorporation into angiogenic matrices in vitro and in vivo.

Durability of hTERT(+)HDMEC in Angiogenic Matrices

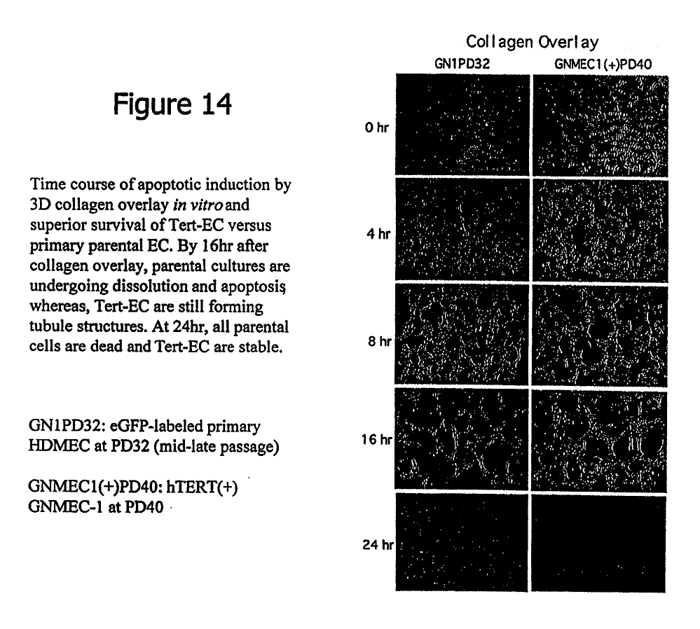

Our previously published results demonstrated that the relative survival advantage of hTERT(+)HDMEC vs primary HDMEC is based upon both life span extension and apoptotic resistance (Yang et al 1999 J. Biol. Chem. 274:37:26141-48). The latter phenomenon was tested by multiple different methods using multiple different apoptotic inducers. However, one class of inducers that was not tested includes extracellular matrix components. In this invention we demonstrate that hTERT(+)HDMEC resist activation of the apoptotic pathway induced by 3D collagen in vitro, a potent and relevant agent (FIG. 14). This robust effect is highly reproducible and consistent with our published results with other inducers. It represents a hurdling the most important barrier to successful human microvascular remodeling assay system development. Taking this to the next level, we then tested the survivability of hTERT(+)HDMEC in vivo and demonstrated their durability was equivalent to or greater than young primary HDMEC (see below).

Fluorescent-labeled hTERT(+)HDMEC

Expression of marker enzymes (e.g. β-galactosidase) or fluorescent proteins (e.g. enhanced green fluorescent protein, eGFP) in primary MEC has rarely been reported due to low gene transduction efficiencies, inability to select and low survival rates. In this invention, we show that retroviral-mediated expression of eGFP in primary HDMEC results in high transduction efficiencies (FIGS. 1, 2). These parental cells (GN1) were then “telomerized” with hTERT, continuously passaged and FAC-sorted by FITC to create a pure population of eGFP-expressing immortalized HDMEC (GNMEC 1). This line reproducibly responds to angiogenic and angiostatic agents in the same manner as early passage parental primary cells and forms the basis of novel in vitro and in vivo microvascular remodeling assay systems.

In Vitro Angiogenic Assay System

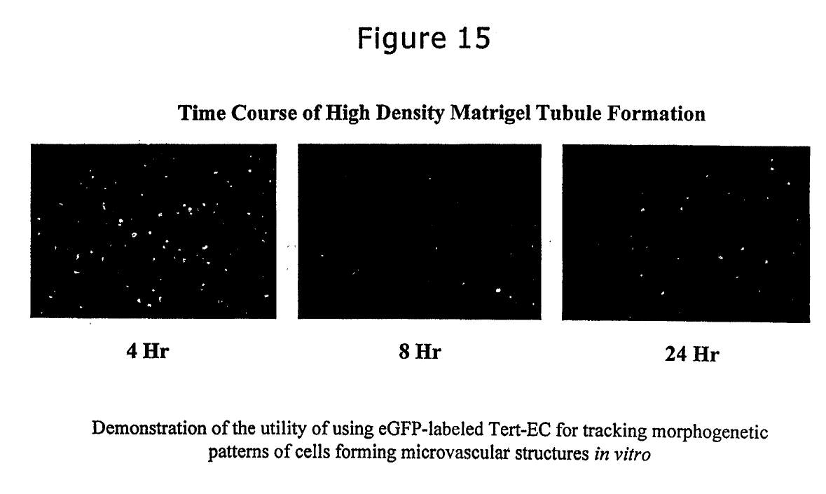

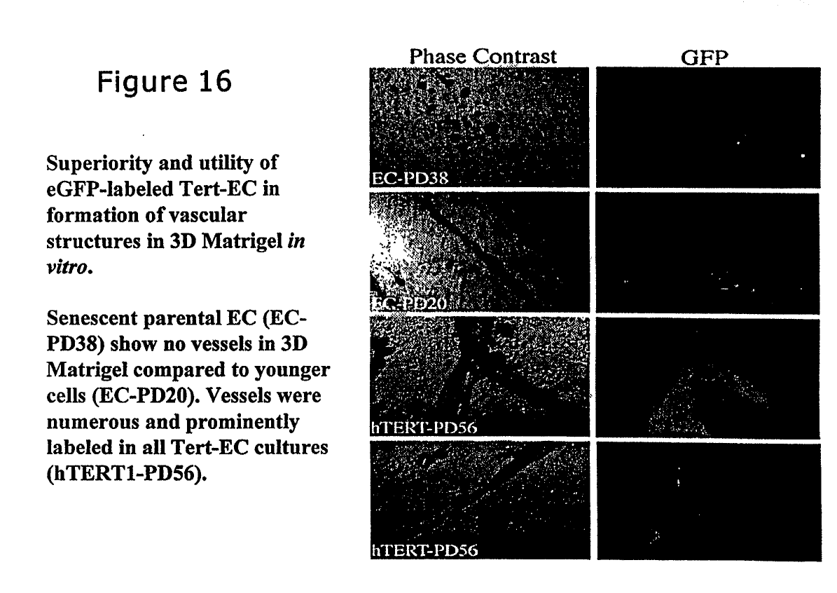

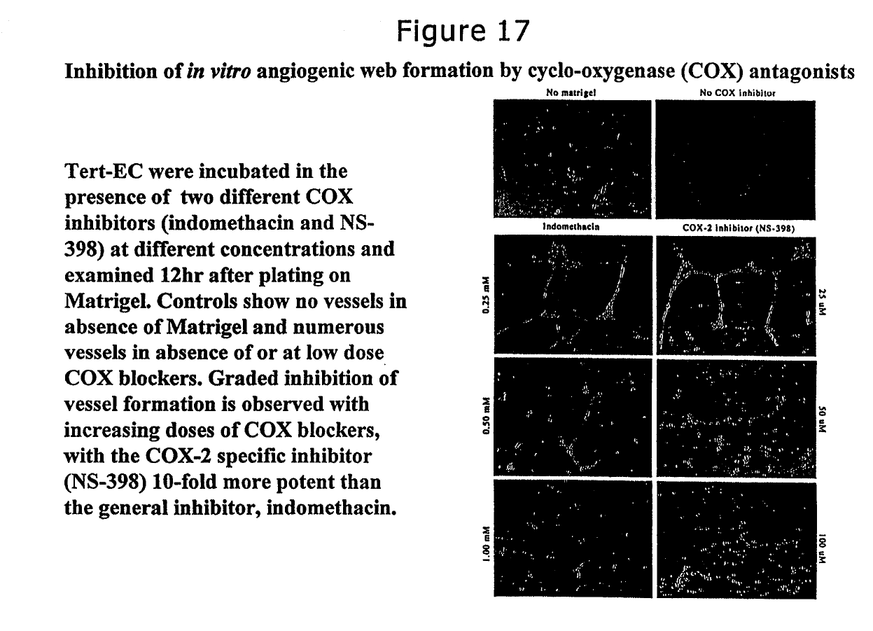

FIG. 15 demonstrates the ability of GNMEC1 to form “angiogenic webs” in response to plating onto a permissive matrix (e.g. Matrigel). FIG. 16 demonstrates the superiority and versatility of using GNMEC1 to form fluorescent vascular structures in 3D Matrigel in vitro versus both senescent or young primary parental cells (GN1). To show the utility of this in vitro system, we pretreated GNMEC1 with two different cyclo-oxygenase (COX) antagonists which are known to block angiogenesis (Jones et al. 1999 Nature Medicine 5:12;1418-23) and then plated the cells on Matrigel. FIG. 17 shows a dose-response curve of angiogenic web blockade by indomethacin and NS-398 to demonstrate the use of hTERT(+)HDMEC to screen therapeutic compounds for their potential efficacy in modulating blood vessel growth. The “tubulogenic process” can be followed and quantified utilizing cell lines such as GNMEC1 and digitally-converted fluorescence microscopic images of replicate cultures. Commercially available programs in current use with other cellular applications which maybe used to perform this process include MetaMorph® (Universal Imaging Corporation®, West Chester, Pa.) and the high-throughput imaging systems of Cellomics, Inc. (Pittsburgh, Pa.).

In Vivo Human Microvascular Remodeling System

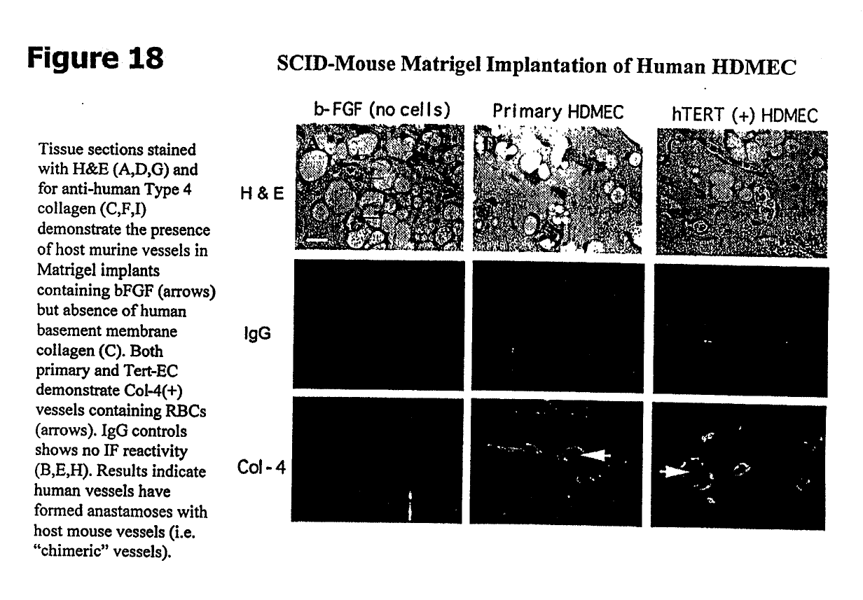

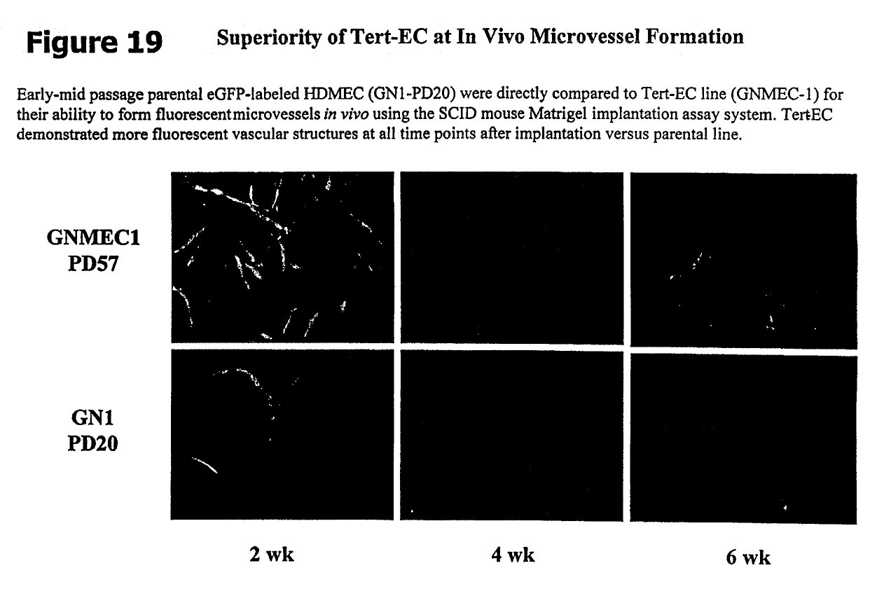

In the present invention proof of principle is demonstrated indicating that hTERT(+)HDMEC can form human blood vessels in vivo. SCID mice were implanted with Matrigel mixtures containing either b-FGF, primary HDMEC (PD1O) or hTERT(+)HDMEC via subcutaneous injection on ventral thoracic surfaces. Implants were harvested at 2 weeks, sectioned and stained with H&E and anti-human basement membrane Type 4 collagen. As shown in FIG. 18, host microvasculature is readily apparent invading matrices containing b-FGF alone but the absence of Type 4 collagen reactivity confirms specificity of murine vs human basement membranes. Immunoreactive lumenal structures in implants containing either primary or hTERT(+)HDMEC demonstrate formation of human microvasculature within the implants. The presence of red blood cells within these human vessels indicates host vasculature has anastamosed with the human vessels creating murine-human chimeric microvasculature.

To prove that the cells we implanted were responsible for the anti-human Type 4 collagen immunoreactivity, we implanted both GN1 (mid passage parental primary HDMEC) and GNMECI in the same SCID system as genetic-tagged cells for rapid identification. Thick sections of implants viewed by UV light demonstrate numerous fluorescent microvessels (FIG. 19) proving that the origin of the vessels were human and demonstrating the utility of using eGFP-labeled hTERT(+)HDMEC in this assay system.

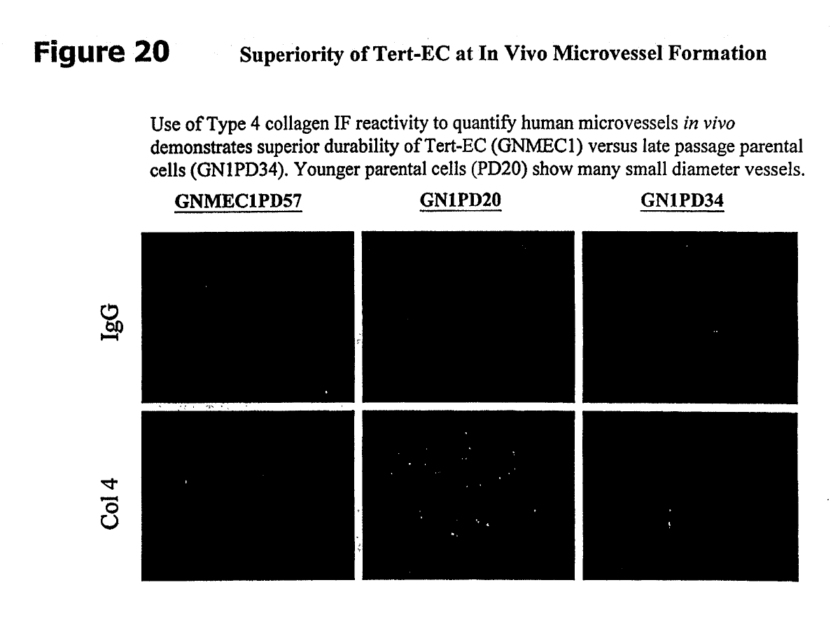

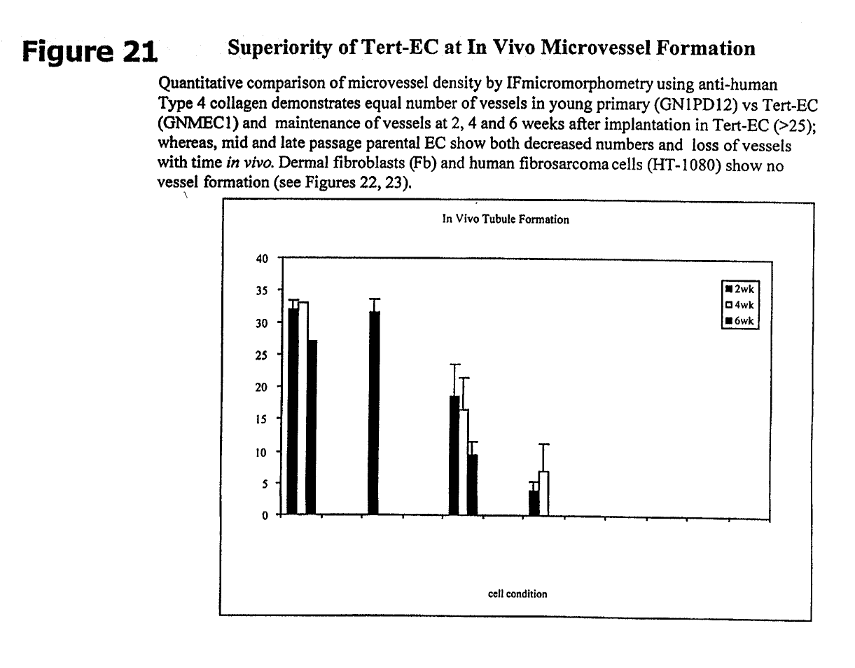

The superior durability of hTERT(+)HDMEC is demonstrated in FIGS. 19-20 in which increased survival of human microvasculature in vivo is apparent versus both mid passage and presenescent primary HDMEC. Quantitative comparison of vessel density using anti-human Type 4 collagen immuno-micromorphometry shows statistically significant and dramatic differences in survival characteristics of hTERT(+)HDMEC with time after implantation in vivo(FIG. 21).

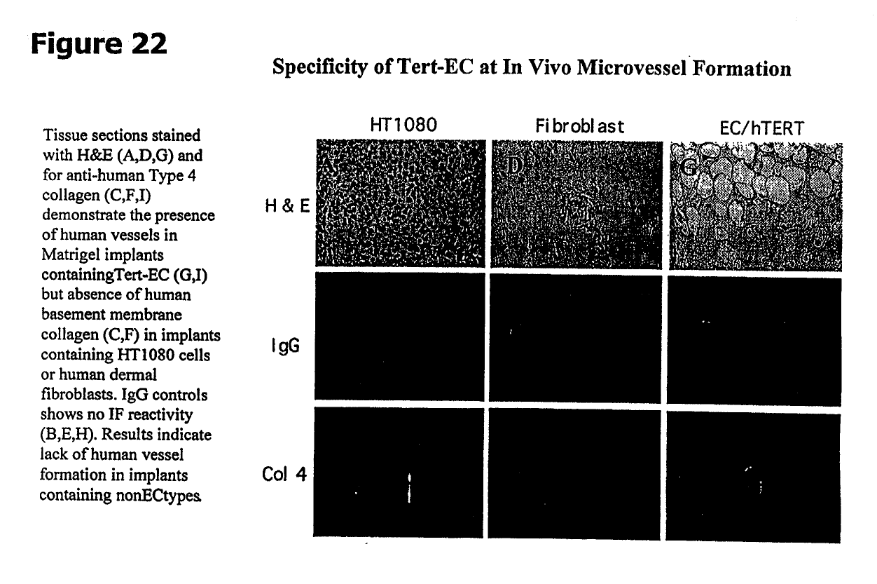

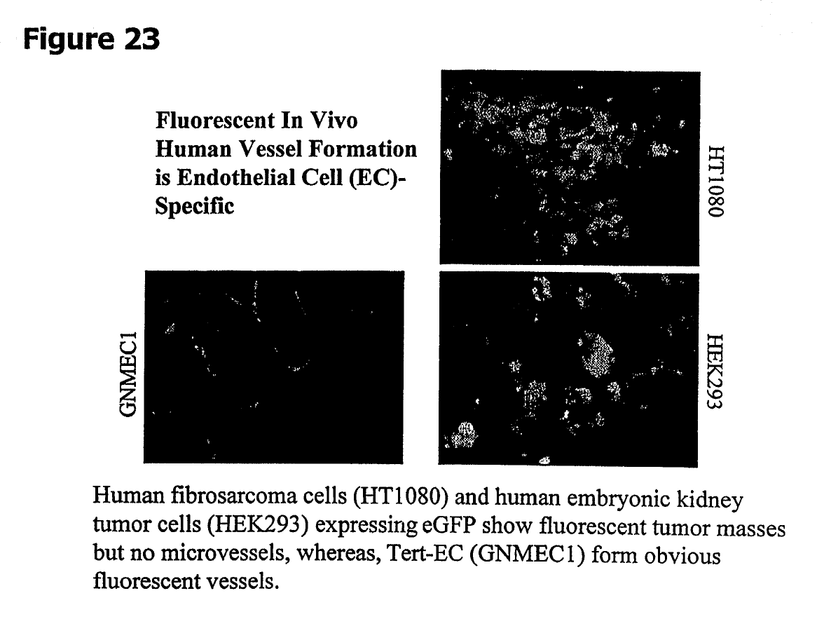

Proof of specificity is demonstrated by substitution of endothelial cells with either human dermal fibroblasts, human fibrosarcoma cells (HT-1080) or human embryonic kidney tumor cells (293) in Matrigel implants. Absence of Type 4 collagen immunoreactivity in these implants is shown in FIG. 22. EGFP-labeled HT1080 and 293 cells demonstrates fluorescent tumor masses in the implant but absence of fluorescent microvasculature (FIG. 23).

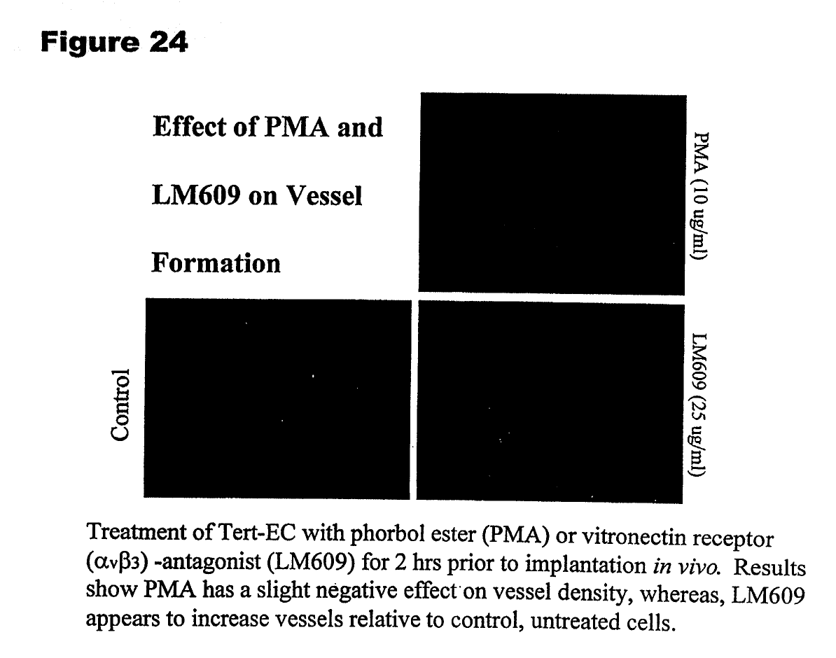

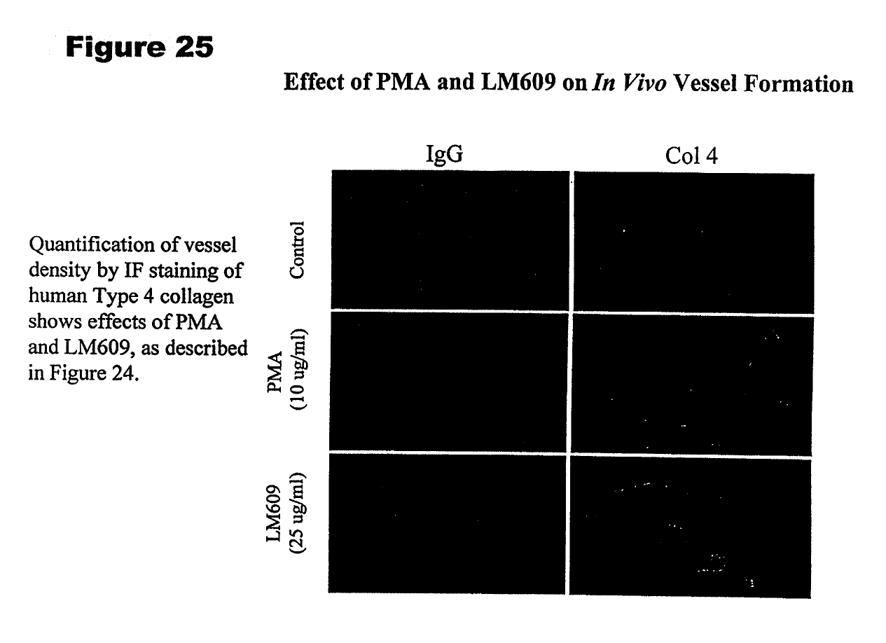

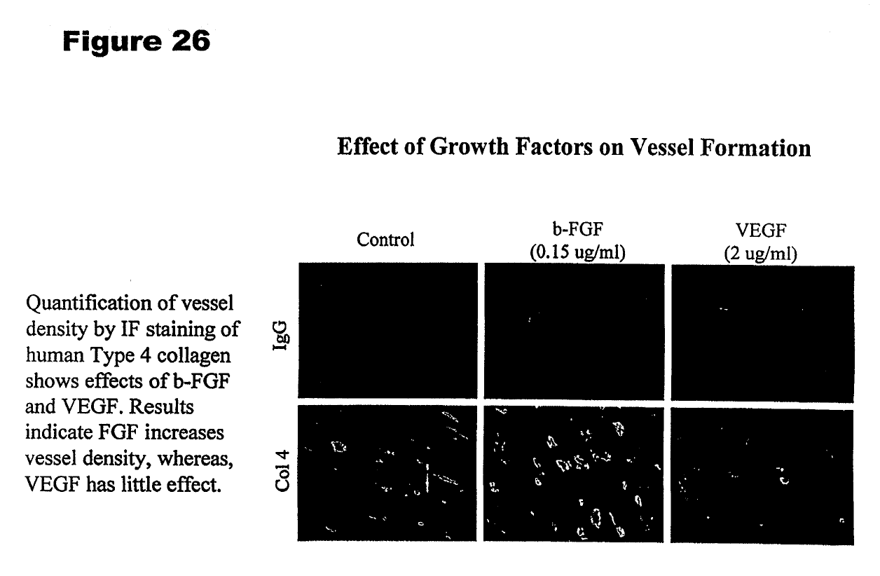

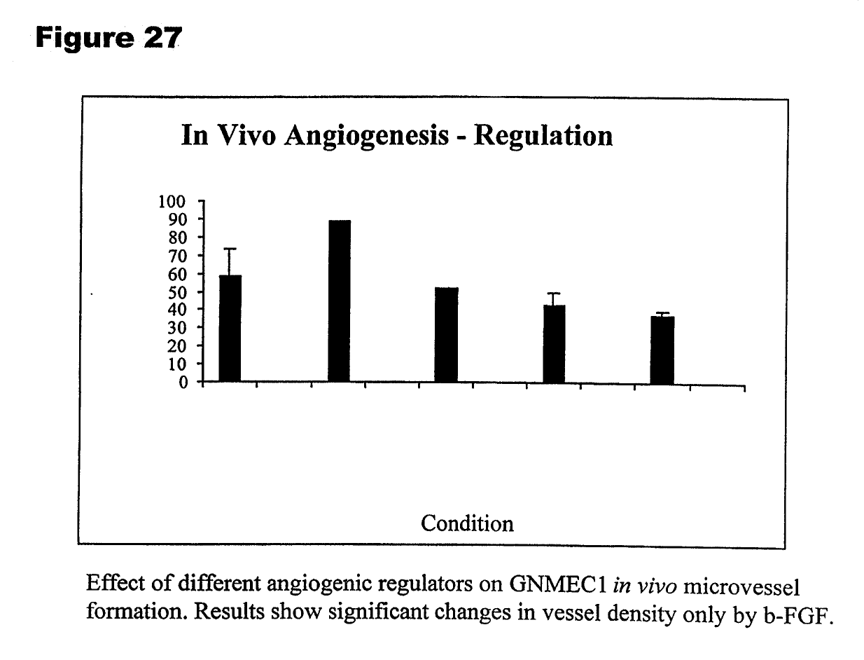

To demonstrate the potential of the in vivo system for testing different agents for their angiogenic and angiostatic qualities we pretreated GNMEC1 with either PMA, b-FGF, VEGF or anti-vitronectin receptor antibody (LM609). FIGS. 24-27 demonstrate variable effects of these agents on human microvessel formation in vivo. The greatest stimulatory effect is observed with b-FGF, whereas, both PMA and LM609 show angiostatic qualities (FIG. 27). All agents were incorporated into Matrigel by mixing cells and agents together before implantation. The effect of continuous exposure at different concentrations was not performed but would be likely to greatly accentuate the results shown in these preliminary studies.

### Example 6

In Vivo Assay System for Identfying Modulators of Angiogenesis

HDMEC Isolation and Culture

The establishment of primary HDMEC was performed by dispase digestion of neonatal foreskin tissue and EC purification using anti-PCAM-1 affinity beads as described 38,39. The references cited herein are described in detail at the end of the description. Primary HDMEC and telomerized EC (HDMEC-T) were cultured in EGM-2-MV medium (Clonetics, San Diego, Calif.). Medium was changed every two days and cells were passaged 1:3. Two primary parental HDMEC populations used in this study were designated HDMEC-1 and HDMEC-G. The latter cells were created by transduction of early passage (PD5) HDMEC with the LZRS retroviral vector expressing eGFP (kindly provided by Helen Deng, Stanford University, Calif.) as described below.

Preparation of Telomerized HDMEC

Plasmid pGRN145 encoding hTERT was provided by Geron Corporation (Menlo Park, Calif.). The hTERT coding region of pGRN145 was subcloned into the LZRS retroviral vector [Romero, L. I., Zhang, D. N., Herron, G. S. & Karasek, M. A. Interleukin-1 induces major phenotypic changes in human skin microvascular endothelial cells. J. Cellular Physiol. 173, 84-92 (1997)] provided by Garry Nolan (Stanford, Calif.). hTERT-LZRS and eGFP-LZRS retroviral particles were produced in the Phoenix packaging cell line (Garry Nolan, Stanford University, Calif.) and both genes were driven by Moloney murine leukemia virus 5′-LTR promoter. Two different HDMEC-T lines were used in this study, HDMEC-T and HDMEC-GT, corresponding to primary parental cell populations, HDMEC-1 and HDMEC-G, respectively. The preparation and characterization of HDMEC-T (aka, hTERT3) was as previously published²¹. An eGFP-labeled telomerized EC population was produced as follows: 1×10⁶ HDMEC-G at population doubling 5 (PD5), were transduced with hTERT-LZRS, allowed to grow without selection for two passages and then sorted for green fluorescence using a BD FACStar to produce HDMEC-GT. HDMEC-T and HDMEC-GT came from two different primary HDMEC and were phenotypically and functionally similar to young primary cells²¹. HDMEC and HDMEC-G had low wild type p16 expression and exogenous hTERT gene transduction did not affect the pattern of its expression. We did not find c-myc activation in any HDMEC-T used in this report and all HDMEC-T were diploid 46, XY. 21. Yang, J. et al. Human endothelial cell life extension by telomerase expression. J. Biol. Chem. 274,26141-26148 (1999).

Assay for Telomerase Activity

Telomerase activity was measured by the TRAP kit from Roche Molecular Biochemicals (Indianapolis, Ind.). Briefly, 2000 cell equivalents were PCR-amplified with a biotin-labeled P1-TS primer. One tenth of the PCR product was run on a 12% non-denaturing acrylamide gel. Following gel electrophoresis, products were transferred and blotted onto a nylon membrane, and processed by the biotin luminescence detection kit (Pharmingen, San Diego, Calif.).

3D in vitro Tubule Formation Assay

1×10⁴ HDMEC-G or HDMEC-GT were mixed with 0.5 ml Matrigel (Beckton Dickinson, Bedford, Mass.) on ice and seeded in each well of a 24 well cluster plate. Plates were imaged one week after seeding by both phase contrast and fluorescence microscopy, images were captured using a CCD camera mounted on a Zeiss Inverted microscope and digitally converted using NIH Image.

SCID Mice Xenografting

This procedure is based on a modification of the mouse angiogenesis model previously described⁴¹. Two-three week old male or female SCID mice (Taconic, Germantown, N.Y.) were used as hosts for all implants. Primary HDMEC and HDMEC-T were harvested, washed twice and re-suspended in serum-free EGM-2 basal medium at the concentration of 1×10⁵/μl. Ten μl of cells were mixed with 0.5 ml of Matrigel on ice and the mixture was implanted in the ventral midline thoracic tissue of each mouse by subcutaneous injection using a #25 needle. Up to three separate injections could be performed on a single mouse. For some experiments, recombinant human VEGF165 (2 μg/ml) (R&D systems, Minneapolis, Minn.) or bovine FGF-2 (150 ng/ml) (R&D systems, Minneapolis, Minn.) were added to the mixture. When tumor cells (HT1080 and 293, ATCC) or primary human dermal fibroblasts 38 were injected, the procedure remained the same except basal DMEM medium replaced EGM-2. 41. Passaniti, A. et al. A simple, quantitative method for assessing angiogenesis and antiangiogenic agents using reconstituted basement membrane, heparin, and fibroblast growth factor. Lab. Invest. 67, 519-528 (1992).

### Example 7

Analyzing the Effect of Angiogenesis Modulators

Thick Section, Whole Mount Tissue Examination

Whole mount Matrigel implants were examined by fluorescence microscopy as follows: The implants were surgically removed from mice after euthanasia by CO₂ asphyxiation, cut into small pieces with a #15 scalpel and further dissected with forceps. Tissues were covered in DABCO mounting medium (Sigma, St. Louis, Mo.) and eGFP signals were captured using the FITC filter on a Zeiss Axioskope microscope equipped with a MC-80 CCD camera. Images were viewed using Adobe Photoshop on a Macintosh Quadra and quantified as described below.

Histology and Human Vessel Quantification

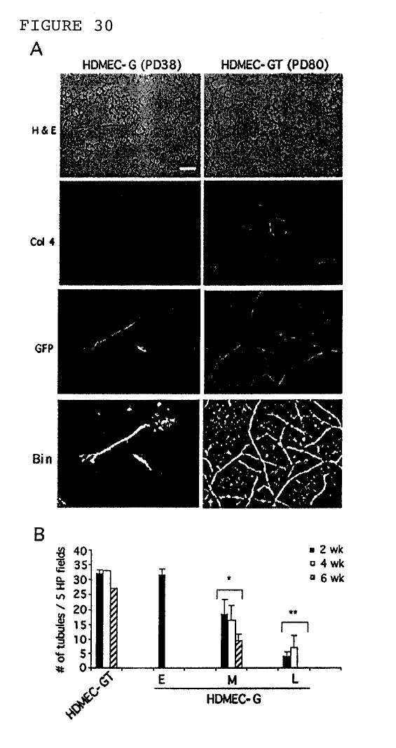

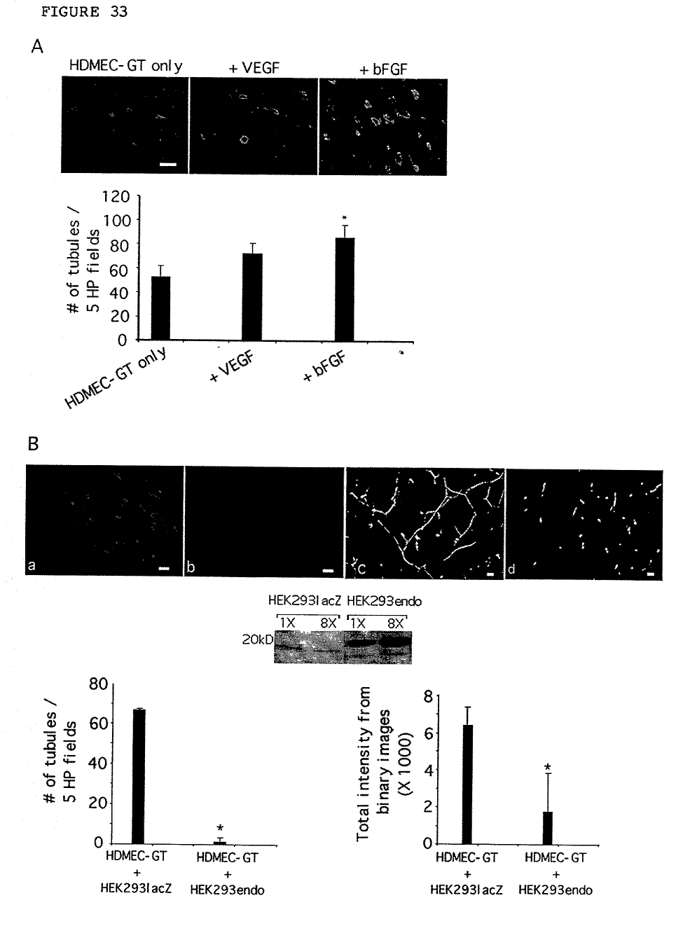

Matrigel implants were removed at 1, 2, 4 and 6 wk following xenografting, fixed in 10% buffered formalin overnight, paraffin-embedded and sectioned. H&E stained thin sections were prepared at Pan-insular Histopathology laboratory (Los Gatos, Calif.). For immunofluorescence, thin sections were deparaffinized and antigens retrieved in 10 mM citric acid (pH 6.0) by microwaving sections for 2×7 min. Sections were then incubated with anti-human type IV collagen IgG (Sigma, St. Louis, Mo.) primary antibody, followed by washing and Cy-3 conjugated secondary IgG according to standard protocols. Immunoreactive human collagen type IV signals were evident as annular and linear structures in all sections containing HDMEC versus both control IgG and sections from implants that did not contain human EC. Implants without FGF-2 or HDMEC contained little or no host microvessels, whereas, marked host vessel invasion was observed in the presence of FGF-2 alone⁴¹. For micromorphometry, 5 separate 20×fields were randomly selected per tissue section and the number of annular structures were counted and averaged. Unless specifically stated otherwise, 3 different sections were viewed per implant and replicate implants were grafted for each experimental condition (FIGS. 30 and 33). Passaniti, A. et al. A simple, quantitative method for assessing angiogenesis and antiangiogenic agents using reconstituted basement membrane, heparin, and fibroblast growth factor. Lab. Invest. 67, 519-528 (1992).

For digital analysis of eGFP fluorescence images we used a novel algorithm (Moss Filter™) to determine the total amount of vascularization in each implant section. This filter determines whether or not each pixel is part of the fluorescently-labeled vascular region. The filter converts the original (8-bit) digital image into a binary image. Pixel values equal to 1 indicate vascularization, whereas zero values indicate no vascularization. The total amount of vascularization in each implant section is obtained by summing all the values in the binary image.

The filter converts the original array of pixel intensities into a new array, called the Discriminant, whose elements describe the likelihood that a particular pixel is part of the vascularized region. For each pixel in the original image, we calculate an element of the Discriminant array. We write:

Element of Discriminant array=Σ_{(row and column)}−(Pixel Intensity−Background)*(Local Curvature)/(|Local Slopel+E where E (a small number) ensures a nonzero denominator. The local curvature and local slope are the second and first derivatives, which are calculated along a row or column for each member of the pixel intensity array. The Discriminant selects locally for a high, peaked, and/or plateaued region (“mountain top”), which is the topological structure of the pixel intensities of the fluorescently-labeled vascular network. The user specifies only a single background pixel intensity and a single numerical threshold for the computed discriminant (how much of a mountain top is desired) for each image. An initial binary image is constructed from all discriminant values that exceed this threshold value. This binary image is refined further by retaining only those pixels that have a value of 1 and have at least two nearest neighbors (each pixel has 8 neighbors) that also have a value of 1. This represents a minimum requirement for connectivity. From this binary, we retain only those pixels that have a value of 1 and have at least three nonzero nearest neighbors. The final binary image is obtained by removing all isolated nonzero valued pixels. FIGS. 30A (Bin) and 33B show representative binary images of the original TIFFs.

Intravascular Tracer Experiments

Mice containing HDMEC-GT xenografts two weeks after implantation were injected with 1.0 μm diameter red fluorescent microspheres (Molecular Probes, Eugene, OR) via tail vein cannulation. After approximately 1 minute, implants were removed and tissues processed as described above for thick section whole mounts. FITC and rhodamine filters were used to visualize eGFP and red microspheres, respectively, and images were captured using either the Zeiss Axioskope or Gen II Multi-dimensional Imager, a fully automated inverted high speed imaging station powered by Universal Imaging Corporation Metamorph™ software.

Endostatin Blocking Experiments

Inhibition of in vivo vessel formation by local delivery of human endostatin was accomplished as follows: The plasmid pGT60hEndo, expressing recombinant human endostatin (InvivoGen, San Diego, Calif.), was stably transfected into human embryonic kidney (HEK293) cell line by calcium phosphate transfection (Invitrogen, Carlsbad, Calif.). Western blot of culture media using endostatin-specific IgG (kind gift from Rupert Timpl, Max Plank Institute, Martinsreid, Germany) showed expression of a 22 kD protein in HEK293endo only. HEK293 cells expressing lacZ served as a control for both Western blots and grafting experiments. The cell implantation procedure was the same as that described above except that 1×10⁵ (or 10⁴ or 10³) transfected HEK were mixed with HDMEC-GT immediately prior to implantation. Grafts were examined at both one week and two after injection and sections were analyzed by both micromorphometry and eGFP as previously described.

### Example 8

Superior Durability of Telomerized Human Dermal Microvascular Endothelial Cells Transduced with eGFP

Creation of eGFP-labeled, Telomerized HDMEC

Our previous studies showed that ectopic expression of recombinant hTERT reconstituted telomerase activity efficiently in human dermal microvascular EC (HDMEC) derived from neonatal foreskin²¹. n the present study, we used both a previously characterized telomerized HDMEC population (HDMEC-T) and a new EC line produced by co-transduction of eGFP and hTERT into HDMEC, called HDMEC-GT. The parental cells used for creating HDMEC-GT were also transduced with eGFP (HDMEC-G). As shown by the TRAP ladder assay, both telomerized EC lines (HDMEC-T and HDMEC-GT) exhibited high telomerase activity, whereas, mid passage parental primary HDMEC (HDMEC, PD25; HDMEC-G, PD28) showed little or no activity. A mass culture of HDMEC-GT with 100% eGFP positively was then produced by FAC sorting (FIG. 28B). The phenotypic and functional properties of this HDMEC-GT subpopulation in vitro were identical to HDMEC-T and both cell populations formed relatively slow growing epitheliod monolayers that expressed all EC markers, including TNF∂-inducible ICAM, VCAM and E-selectin. 21. Yang, J. et al. Human endothelial cell life extension by telomerase expression. J. Biol. Chem. 274,26141-26148 (1999).

In vitro Tubule Formation

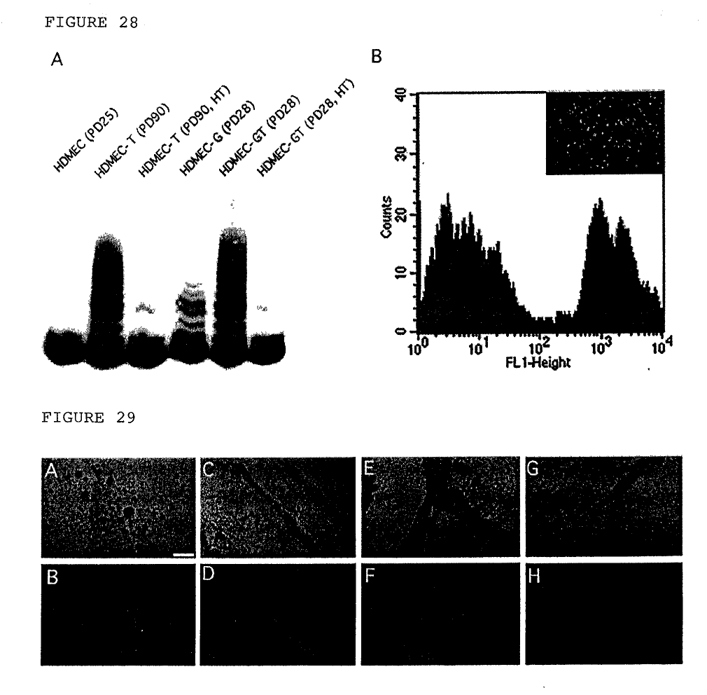

The functionality of HDMEC-GT was also assessed by tracking morphogenetic movements of cells in a “permissive” matrix environment in vitro. As shown in FIG. 29 the formation of tubule structures in 3D Matrigel using both parental primary cells and HDMEC-GT was visualized by phase contrast and fluorescence microscopy. Similar to pre-senescent primary human umbilical vein endothelial cells (HUVEC) seeded atop Matrigel 21 we found that pre-senescent primary HDMEC-G (PD38) did not form tubules in 3-D Matrigel (FIGS. 29A, 29B) but mid-passage (PD20) HDMEC-G did. However, we noted that both the number and branching of HDMEC-G tubule structures were diminished (FIGS. 29C, 29D) relative to HDMEC-GT which formed tubules with strong eGFP signals (FIGS. 29F, 29H) and abundant branching (FIGS. 29E, 29G). HDMEC-GT were used at twice the replicative age (PD56) of senescent primary cells (˜PD25-30). These results suggest that telomerized, eGFP-labeled HDMEC may have an advantage in forming genetically-tagged vascular structures in vivo.

Persistence of Telomerized EC In Vivo

We subcutaneously implanted both HDMEC-GT and in vitro aged HDMEC as 3D Matrigel xenografts in SCID mice and analyzed the grafts at 2, 4 and 6 wk after implantation. FIG. 30A shows representative H&E, eGFP fluorescent images and digitized fluorescent images (Bin) of HDMEC (PD38) and HDMEC-GT (PD56) 2 weeks after xenografting. While H & E staining did not reveal major differences, both grafts showed some areas containing cystic spaces and lymphocyte infiltration and other areas where clear endothelial-lined spaces containing red blood cells were evident. Direct immunofluorescence microscopy using anti-human type IV collagen immunoreactivity in thin sections revealed bright circular and linear structures in the HDMEC-GT-containing implants, but not in implants containing PD38 parental cells (FIG. 30A, Col 4 images). Combined with the H&E results, this suggested that the implants contained a mixture of both host murine and human vessels. The human origin of these structures was confirmed by fluorescence microscopy of implant thick sections that showed bright green tubular structures in HDMEC-GT grafts (FIG. 30A, GFP images). We also used a digital image program (Moss Filter™) to enhance visualization of these fluorescent vessels (FIG. 30A, Bin). eGFP expression correlated well with Col 4 immunoreactivity in young primary HDMEC-G (PD<15) and HDMEC-GT independent of PD; however, we noted that eGFP fluorescence signal intensity was inversely correlated with PD in primary cells. Thus, in vitro aged HDMEC-G had weaker eGFP signals relative to HDMEC-GT (e.g. FIGS. 29C-D vs. 29E-H). These results were consistent in multiple different experiments using over 50 mice, each with up to three implants.

Because primary HDMEC-G did not maintain eGFP fluorescence with time we used micromorphometry of anti-human type IV collagen immunoreactivity (counting lumenal/circular structures per 5 high power fields in thin sections) to quantify human vessel density in the implants from both primary and telomerized EC. FIG. 30B shows that while both mid (PD20, M) and late (PD40, L) passage primary HDMEC exhibited decreased vessel density with time after implantation, telomerized vessels were maintained at about the level of early passage (PD 12, E) primary cells. Due to the lack of sufficient numbers of the latter cells we were unable to test the long-term survival of early passage parental primary cells in vivo. However, mid- and late-passage parental HDMEC showed statistically significant lower vessel densities relative to that of telomerized HDMEC implants, (p<0.01 and p<0.001, respectively. n-3 each).

### Example 9

In Vivo Vessel Formation is EC Specific

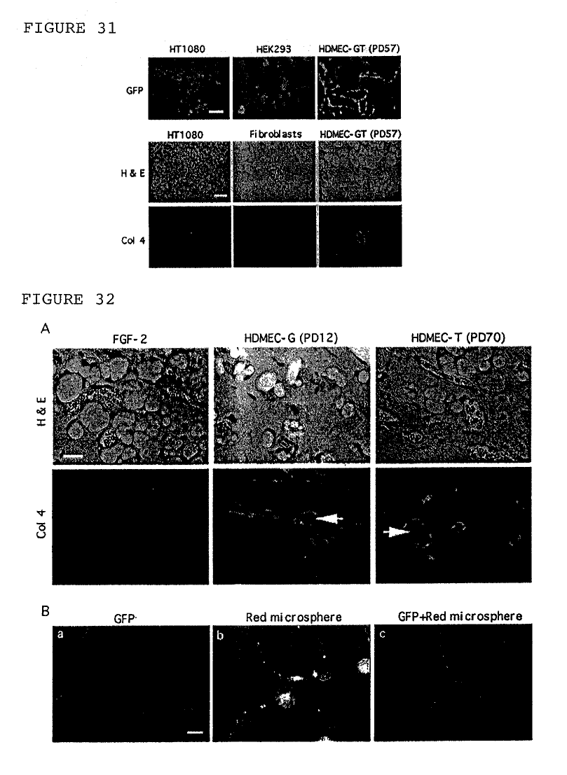

To prove that formation of these human vessel structures in SCID mouse xenografts was a property of EC but not other cells, human fibrosarcoma cells (HT1080), embryonic kidney (HEK293) cells, or primary human dermal fibroblasts were xenografted in duplicate animals under identical conditions as telomerized HDMEC in SCID mice. Two weeks after implantation of eGFP-transduced tumor cells, sections of implants showed HDMEC-GT formed tubular networks while HT1080 and HEK293 formed solid, fluorescent tumor masses (FIG. 31, upper panel). Type IV collagen immunoreactivity showed no evidence of lumenal structures in HT1080 or fibroblast implants (FIG. 31, lower panels).

Recent demonstration of vascular mimicry using melanoma cells in vitro and in vivo suggests that while EC may not be the only cell type capable of forming vascular structures^{22,23} we show the absolute requirement for human EC in our in vivo model of human vessel formation. However, since FIG. 29 showed that tubule structures could be formed in 3D Matrigel in vitro without implantation in SCID mice, we determined whether these capillary structures formed in vivo could function as living blood vessels in SCID mice. 22. Maniotis, A. J. et al. Vascular channel formation by human melanoma cells in vivo and in vitro: vasculogenic mimicry. Am. J. Pathol. 155, 739-752 (1999). 23. Folberg, R., Hendrix, M. J. & Maniotis, A. J. Vasculogenic mimicry and tumor angiogenesis. Am. J. Pathol. 156, 361-81(2000).

Functional human vessels carry host mouse blood. Previous work has shown that an angiogenic factor (e.g. FGF-2) incorporated into Matrigel implants in SCID mice was sufficient to allow invasion of host murine blood vessels 24 FIG. 32A demonstrates this effect in the absence of human EC (upper left panel). However whenever human HDMEC (primary or telomerized) were engrafted in SCID mice as Matrigel implants in the absence of FGF-2, we found anti-human type IV collagen immunoreactive vascular structures that contained lumenal red blood cells (FIG. 32 upper middle and right panels). Given that type IV collagen immunoreactivity associates with eGFP fluorescence (FIGS. 30 and 31), the appearance of host blood cells within these vessels strongly suggests that anastomoses have formed between human and mouse vessels. However, it is possible that post-mortem surgical manipulation of implants may have resulted in artifactual contamination or spillage of blood across tissue sections.

To demonstrate functional murine-human vessel communication we directly delivered an intravascular tracer (red fluorescence microspheres) into the host circulation via tail vein cannulation and found that the tracer localized within eGFP-labeled, human vascular structures one minute after injection (FIG. 32B). The proportion of human vessels that contained the tracer varied between approximately 5% to 50% of total eGFP-labeled vessels in multiple experiments. The majority of implants showed host vessels contained varying amounts of the tracer. Red signals adjacent to eGFP-labeled vessels (Fig B, panel b) suggested that vascular leakage from these newly formed human vessels had occurred. Since we initially examined host-human vessel communication at two weeks after EC implantation, it is likely that the leakage phenomenon may be different at later time points, as vessels ‘mature’ in vivo. Recent studies indicate that murine-human chimeric microvessels are detectable within one month of xenografting primary human EC over-expressing bcl-2 in SCID mice and it is possible that host perivascular support cells (i.e. pericytes) contribute to stabilization of human vessels thereby decreasing vascular leakage at later time points³. 3. Schechner, J. et al. In vivo formation of complex microvessels lined by human endothelial cells in an immunodeficient mouse. Proc. Natl. Acad. Sci USA. 97, 9191-9196 (2000).

These results support and extend our previous in vitro studies that showed a survival advantage of HDMEC-T relative to aged primary HDMEC²¹. Although telomerase life-extended cells have been used recently to engineer functional tissues in vivo^{25,26}, here we show that telomerized human blood vessels can be grown in SCID mice and communicate with the host circulatory system. Furthermore, by directly comparing in vitro-aged primary parental EC to HDMEC-T our results demonstrate for the first time that telomerase activation in human EC results in the maintenance of a stable microvascular phenotype in vivo. Importantly, implanted telomerized EC did not result in tumor formation up to six weeks after implantation, consistent with previous studies of hTERT-transduced primary cells^{21,27,28}. 21. Yang, J. et al. Human endothelial cell life extension by telomerase expression. J. Biol. Chem. 274,26141-26148 (1999). 25. Shay, J. W. & Wright, W. E. The use of telomerized cells for tissue engineering. Nat. Biotechnol. 18,22-23 (2000). 26. Thomas, M., Yang, L. & Hornsby, P. J. Formation of functional tissue from transplanted adrenocortical cells expressing telomerase reverse transcriptase. Nat. Biotechnol. 18, 39-42 (2000). 27. Jiang, X. et al. Telomerase expression in human somatic cells does not induce changes associated with a transformed phenotype. Nat. Genet. 21, 111-114 (1999). 28. Morales, C. et al. Absence of cancer-associated changes in human fibroblasts immortalized with telomerase. Nat. Genet. 21, 115-118 (1999).

Since HDMEC-T were originally isolated from neonatal dermal microvessels, then dispersed cells allowed to reform vascular structures within Matrigel implants, this SCID-human capillary blood vessel model appears to exhibit elements of both intussusception and vascular remodeling in vivo²⁹⁻³¹. However, we have not demonstrated all known steps of angiogenesis nor characterized the angiogenic program of HDMEC-GT in vivo. While an intriguing possibility to consider, it remains to be shown whether a small subpopulation of bone marrow derived EC precursors (e.g. angioblasts), present in the neonatal HDMEC cultures we transduced with hTERT, could be contributing a ‘vasculogenic’ response in this model system^{31,35}. Characterizing and testing different FAC-sorted HDMEC-T populations using our in vivo system may help to clarify potential involvement of such EC precursor populations. 29. Carmeliet, P. Mechanisms of angiogenesis and arteriogenesis. Nat. Med. 6, 389-395 (2000). 30. Helmlinger, G., Endo, M., Ferrara, N., Hlatky, L. & Jain, R. K. Growth factors: formation of endothelial cell networks. Nature 405, 139-141 (2000). 31. Risau, W. Mechanisms of angiogenesis. Nature 386, 671-674 (1997). 32. Asahara T. et al. Isolation of putative progenitor endothelial cells for angiogenesis. Science 275, 964-967 (1997). 33. Shi, Q. et al. Evidence for Circulating Bone Marrow-Derived Endothelial Cells. Blood 92, 362-367 (1998). 34. Takahashi, T. et al. Ischemia- and cytokine-induced mobilization of bone marrow-derived endothelial progenitor cells for neovascularization. Nat. Med. 5, 434-438 (1999). 35. Kalka, C. et al. Transplantation of ex vivo expanded endothelial progenitor cells for therapeutic neovascularization. Proc. Natl. Acad. Sci USA. 97, 3422-3427 (2000).

### Example 10

In Vivo Vessel Density Correlates with Pro-Angiogenic and Angiostatic Factors

In vivo angiogenesis models have been continuously developed during the past 30 years^{4,24}. Most of these models evaluate new blood vessel formation based on the growth of host animal capillaries in response to a controlled microenvironment. More recently, normal human tissue or cancer cell lines have been xenografted in SCID mice for studies of would healing and tumors^{36,37}. In order to test whether HDMEC-T-derived microvascular networks could be modulated by known pro-angiogenic factors, VEGF or FGF-2 were mixed with cells and Matrigel before implantation. Using human type IV collagen micromorphometry, we found statistically increased human vessel density two weeks after grafting HDMEC-GT with FGF-2 (FIG. 33A). While VEGF showed a 20-30% increased vessel density relative to controls, micromorphometry did not demonstrate statistical significance. 4. Jain, R. K., Schlenger, K., Höckel, M. & Yuan, F. Quantitative angiogenesis assays: progress and problems. Nat. Med. 3, 1203-1208 (1997). 24. Capogrossi, M. C. & Passaniti, A. An in vivo angiogenesis assay to study positive and negative regulators of neovascularization. Methods in Molecular Medicine (1998). 36. Christofidou-Solomidou, M., Bridges, M., Murphy, G. F., Albelda, S. M. & DeLisser, H. M. Expression and function of endothelial cell alpha v integrin receptors in wound-induced human angiogenesis in human skin/SCID mice chimeras. Am. J. Pathol. 151, 975-983 (1997). 37. Yuan, F. et al Time-dependent vascular regression and permeability changes in established human tumor xenografts induced by an anti-vascular endothelial growth factor/vascular permeability factor antibody. Proc. Natl. Acad. Sci USA. 93, 14765-14770 (1996).

To test the effect of potential angiogenic blocking agents in this model, a 1:10 ratio of 293 cells expressing endostatin cDNA (HEK293endo) was mixed with HDMEC-GT together with Matrigel immediately before implanting in SCID mice. Implants removed after both one and two weeks demonstrated dispersed, fluorescent spindle-shaped and round cells in grafts from endostatin tissue versus sham transfected (293HEKlacZ) controls (FIGS. 33B; D vs. C). Morphometric analysis and digital quantification using total fluorescence intensity extracted from binary images (FIG. 33B lower bar graphs) demonstrated statistically significant loss of vessel density in HEK293endo implants, confirming the morphologic appearance of these tissues.

In summary, we have established a system for studying the mechanisms of human microvessel formation in a controlled experimental setting in vitro and in vivo. Our in vivo model or system relies on the superior survival and uniformity of HDMEC-GT, and is specific and quantitative. Telomerized, genetically-tagged human EC respond appropriately to both pro-angiogenic and angiostatic factors by modulating vessel density in vivo. While we reported that our telomerized EC populations resist apoptotic induction relative only to in vitro aged primary parental EC populations²¹ the potential for altered apoptotic signaling in telomerized EC lines in vivo may impact the ability of our model to mimic the exact responses of primary HDMEC and/or dermal capillaries in human tissues. Nevertheless, this system does not depend on constitutive blockade of apoptotic signal transduction pathways via enforced bcl-2 expression^{2,3} and thus it provides a superior platform for testing the effects of agents that may modulate EC programmed cell death. Such characteristics are required for preclinical drug screening programs and our model may be utilized in the design of engineered human vascular tissues that will facilitate surgical grafting, vascular implantation, chronic wound management and clarification of tumor angiogenesis^{33,34}. 2. Nör, J., Joan, C., Mooney, D. & Polverini, P. Vascular Endothelial Growth Factor (VEGF)—Mediated Angiogenesis is Associated With Enhanced Endothelial Cell Survival and induction of Bcl-2 Expression. Am. J. Pathol. 154, 375-384 (1999). 3. Schechner, J. et al. In vivo formation of complex microvessels lined by human endothelial cells in an immunodeficient mouse. Proc. Natl. Acad. Sci USA. 97, 9191-9196 (2000). 33. Shi, Q. et al. Evidence for Circulating Bone Marrow-Derived Endothelial Cells. Blood 92, 362-367 (1998). 34. Takahashi, T. et al. Ischemia- and cytokine-induced mobilization of bone marrow-derived endothelial progenitor cells for neovascularization. Nat. Med. 5, 434-438 (1999).

Although preferred embodiments of the subject invention have been described in some detail, it is understood that obvious variations can be made without departing from the spirit and the scope of the invention as defined by the appended claims.

References

The following references are hereby incorporated by reference in their entirety.

1. L'Heureux N., Paquet S., Labbe R., Germain L. & Auger F. A. A completely biological tissue-engineered human blood vessel. FASEB J. 12, 47-56 (1998).

2. Nör, J., Joan, C., Mooney, D. & Polverini, P. Vascular Endothelial Growth Factor (VEGF)—Mediated Angiogenesis is Associated With Enhanced Endothelial Cell Survival and induction of Bcl-2 Expression. Am. J. Pathol. 154, 375-384 (1999).

3. Schechner, J. et al. In vivo formation of complex microvessels lined by human endothelial cells in an immunodeficient mouse. Proc. Natl. Acad. Sci USA. 97, 9191-9196 (2000).

4. Jain, R. K., Schlenger, K., Höckel, M. & Yuan, F. Quantitative angiogenesis assays: progress and problems. Nat. Med. 3, 1203-1208 (1997).

5. Palmer, R., Ferrige, A. & Moncada, S. Nitric Oxide release accounts for the biological activity of EDRF. Nature 327, 524-526 (1987).

6. Pili, R. et al. Altered angiogenesis underlying age-dependent changes in tumor growth. J. Natl. Cancer Inst. 86, 1303-1314 (1994).

7. Reed, M. J., Corsa, A. C., Kudravi, S. A., McCormick, R. S. & Arthur, W. T. A deficit in collagenase activity contributes to impaired migration of aged microvascular endothelial cells. J. Cellular Biochem. 77, 116-126 (2000).

8. Rivard, A. et al. Age-dependent impairment of angiogenesis. Circulation 99, 111-120 (1999).

9. Swift, M. E., Kleinman, H. K. & DiPietro, L. A. Impaired wound repair and delayed angiogenesis in aged mice. Lab. Invest. 79, 1479-1487 (1999).

10. Whikehart, D. R., Register, S. J., Chang, Q. & Montgomery, B. Relationship of telomeres and p53 in aging bovine corneal endothelial cell cultures. Invest. Ophthal. & Vis. Sci. 41, 1070-1075 (2000).

11. Chang, E. & Harley, C. B. Telomere length and replicative aging in human vascular tissues. Proc. Natl. Acad. Sci USA. 92, 11190-11194 (1995).

12. Maier, J. A., Voulalas, P., Roeder, D. & Maciag, T. Extension of the life-span of human endothelial cells by an interleukin-1 alpha antisense oligomer. Science 249, 1570-1574 (1990).

13. Watanabe, Y., Lee, S. W., Detmar, M., Ajioka, I. & Dvorak, H. F. Vascular permeability factor/vascular endothelial growth factor (VPF/VEGF) delays and induces escape from senescence in human dermal microvascular endothelial cells. Oncogene 14, 2025-2032 (1997).

14. Shelton, D. N., Chang, E., Whittier, P. S., Choi, D. & Funk, W. D. Microarray Analysis of replicative senescence. Cur. Biol. 9, 939-945 (1999).

15. Ades, E. W. et al. HMEC-1 Establishment of an Immortalized Human Microvascular Endothelial Cell Line. J. Invest. Dermatol. 99, 683-690 (1992).

16. Dubois, N. A., Kolpack, L. C., Wang, R., Azizkhan, R. G. & Bautch, V. L. Isolation and characterization of an established endothelial cell line from transgenic mouse hemangiomas. Exp. Cell Res. 196, 302-313 (1991).

17. Candal, F. J. et al. BMEC-1: a human bone marrow microvascular endothelial cell line with primary cell characteristics. Microvasc. Res. 52, 221-234 (1996).

18. Fontijn, R. et al. Maintenance of vascular endothelial cell-specific properties after immortalization with an amphotrophic replication-deficient retrovirus containing human papilloma virus 16 E6/E7 DNA. Exp. Cell Res. 216, 199-207 (1995).

19. Cockerill, G. W., Meyer, G., Noack, L., Vadas, M. A. & Gamble, J. R. Characterization of a spontaneously transformed human endothelial cell line. Lab. Invest. 71, 497-509 (1994).

20. Rhim, J. S. et al. A human vascular endothelial cell model to study angiogenesis and tumorigenesis. Carcinogenesis 19, 673-681 (1998).

21. Yang, J. et al. Human endothelial cell life extension by telomerase expression. J. Biol. Chem. 274,26141-26148 (1999).

22. Maniotis, A. J. et al. Vascular channel formation by human melanoma cells in vivo and in vitro: vasculogenic mimicry. Am. J. Pathol. 155, 739-752 (1999).

23. Folberg, R., Hendrix, M. J. & Maniotis, A. J. Vasculogenic mimicry and tumor angiogenesis. Am. J. Pathol. 156, 361-81(2000).

24. Capogrossi, M. C. & Passaniti, A. An in vivo angiogenesis assay to study positive and negative regulators of neovascularization. Methods in Molecular Medicine (1998).

25. Shay, J. W. & Wright, W. E. The use of telomerized cells for tissue engineering. Nat. Biotechnol. 18,22-23 (2000).

26. Thomas, M., Yang, L. & Hornsby, P. J. Formation of functional tissue from transplanted adrenocortical cells expressing telomerase reverse transcriptase. Nat. Biotechnol. 18, 39-42 (2000).

27. Jiang, X. et al. Telomerase expression in human somatic cells does not induce changes associated with a transformed phenotype. Nat. Genet. 21, 111-114 (1999).

28. Morales, C. et al. Absence of cancer-associated changes in human fibroblasts immortalized with telomerase. Nat. Genet. 21, 115-118 (1999).

29. Carmeliet, P. Mechanisms of angiogenesis and arteriogenesis. Nat. Med. 6, 389-395 (2000).

30. Helmlinger, G., Endo, M., Ferrara, N., Hlatky, L. & Jain, R. K. Growth factors: formation of endothelial cell networks. Nature 405, 139-141 (2000).

31. Risau, W. Mechanisms of angiogenesis. Nature 386, 671-674 (1997).

32. Asahara T. et al. Isolation of putative progenitor endothelial cells for angiogenesis. Science 275, 964-967 (1997).

33. Shi, Q. et al. Evidence for Circulating Bone Marrow-Derived Endothelial Cells. Blood 92, 362-367 (1998).

34. Takahashi, T. et al. Ischemia- and cytokine-induced mobilization of bone marrow-derived endothelial progenitor cells for neovascularization. Nat. Med. 5, 434-438 (1999).

35. Kalka, C. et al. Transplantation of ex vivo expanded endothelial progenitor cells for therapeutic neovascularization. Proc. Natl. Acad. Sci USA. 97, 3422-3427 (2000).

36. Christofidou-Solomidou, M., Bridges, M., Murphy, G. F., Albelda, S. M. & DeLisser, H.M. Expression and function of endothelial cell alpha v integrin receptors in wound-induced human angiogenesis in human skin/SCID mice chimeras. Am. J. Pathol. 151, 975-983 (1997).

37. Yuan, F. et al Time-dependent vascular regression and permeability changes in established human tumor xenografts induced by an anti-vascular endothelial growth factor/vascular permeability factor antibody. Proc. Natl. Acad. Sci USA. 93, 14765-14770 (1996).

38. Normand, J. & Karasek, M. A. A method for the isolation and serial propagation of keratinocytes, endothelial cells, and fibroblasts from a single punch biopsy of human skin. In Vitro Cellular & Dev. Biol. Animal 31, 447-455 (1995).

39. Romero, L. I., Zhang, D. N., Herron, G. S. & Karasek, M. A. Interleukin-1 induces major phenotypic changes in human skin microvascular endothelial cells. J. Cellular Physiol. 173, 84-92 (1997).

40. Kinsella, T. M. & Nolan, G. P. Episomal vectors rapidly and stably produce high-titer recombinant retrovirus. Hum. Gene Ther. 7, 1405-1413 (1996).

41. Passaniti, A. et al. A simple, quantitative method for assessing angiogenesis and antiangiogenic agents using reconstituted basement membrane, heparin, and fibroblast growth factor. Lab. Invest. 67, 519-528 (1992).

1. A composition of endothelial cells comprising immortal microvascular endothelial cells, said cells each comprising a recombinant expression cassette encoding telomerase, wherein said cells (a) have a normal karyotype, (b) are resistant to apoptosis relative to primary microvascular endothelial cells, and (c) are not transformed.

2. The composition of claim 1, wherein said cells are human dermal microvascular endothelial cells.

3. The composition of claim 1, wherein said telomerase is a human telomerase reverse transcriptase catalytic subunit.

4. The composition of claim 1, wherein said cells express one or more phenotypic traits expressed uniquely by young primary microvascular endothelial cells.

5. The composition of claim 4, wherein said phenotypic trait is selected from the group consisting of surface receptors, signaling pathways, and both.

6. The composition of claim 1, wherein said cells stably express a transformed genetic marker.

7. The composition of claim 6, wherein said transformed genetic marker is enhanced green fluorescent protein (eGFP).

8. The composition of claim 7, wherein said cells form human microvascular structures in vitro.

9. The composition of claim 8, wherein said human microvascular structures are quantifiable with digital imaging.

10. The composition of claim 9, wherein said digital imaging is fluorescence digital imaging.

11. The composition of claim 8, wherein growth of said human microvascular structures is modulated by a pharmaceutically acceptable compound.

12. The composition of claim 11, wherein said compound promotes angiogenesis.

13. The composition of claim 12, wherein said compound is VEGF.

14. The composition of claim 12, wherein said compound is FGF-2.

15. The composition of claim 11, wherein said compound is an anti-angiogenic compound.

16. The composition of claim 15, wherein said anti-angiogenic compound is endostatin.

17. The composition of claim 7, wherein said cells form human microvascular structures in vivo.

18. The composition of claim 17, wherein said human microvascular structures are quantifiable with digital imaging.

19. The composition of claim 18, wherein said digital imaging is fluorescence digital imaging.

20. The composition of claim 17, wherein growth of said human microvascular structures is modulated by a pharmaceutically acceptable compound.

21. The composition of claim 20, wherein said compound promotes angiogenesis.

22. The composition of claim 21, wherein said compound is VEGF.

23. The composition of claim 21, wherein said compound is FGF-2.

24. The composition of claim 20, wherein said compound is an anti-angiogenic compound.

25. The composition of claim 24, wherein said anti-angiogenic compound is endostatin.

26. The composition of claim 1, wherein said cells form human microvascular structures in vitro.

27. The composition of claim 1, wherein said cells form human microvascular structures in vivo.

28. The composition of any one of claims 1 to 27, wherein said cells demonstrate an extension of cellular life span and resistance to apoptosis comparable to young primary human dermal microvascular endothelial cells.

29. The composition of claim 28, wherein said cells demonstrate said extended cellular life span and resistance to apoptosis in vivo using a SCID-Human Chimeric Microvascular Remodeling Assay System.

30. A composition of endothelial cells comprising immortal microvascular endothelial cells, wherein said cells each stably express enhanced green fluorescent protein (eGFP) and comprise a recombinant expression cassette encoding telomerase, wherein said cells (a) have a normal karyotype, (b) are resistant to apoptosis relative to primary microvascular endothelial cells, and (c) are not transformed.

31. A method of producing a composition of endothelial cells comprising immortal microvascular endothelial cells, wherein said cells each comprise a recombinant expression cassette encoding telomerase, wherein said cells (a) have a normal karyotype, (b) are resistant to apoptosis relative to primary microvascular endothelial cells, and (c) are not transformed, comprising introducing said recombinant expression cassette encoding telomerase into human dermal microvascular endothelial cells and expressing said telomerase.

32. A composition produced by the method of claim 31, wherein said microvascular cells form neovasculature, and wherein host blood is transmitted through said neovasculature.

33. A composition produced by the method of claim 31, wherein said microvascular cells form neovasculature in vivo, and wherein host blood is transmitted through said neovasculature.

34. A composition comprising microvascular cells, wherein said cells form neovasculature, and wherein host blood is transmitted through said neovasculature.

35. The composition of claim 34, wherein said cells form neovasculature in vivo.

36. The composition of claim 34, wherein said cells comprise a genetic marker, wherein said marker is expressible in said cells; and wherein said marker is introduced into said cells through a molecule of recombinant DNA.

37. The composition of any one of claims 32 to 36, wherein said neovasculature is human and wherein said in vivo host is non-human.

38. The composition of claim 37, wherein said in vivo host is a non-human mammal.

39. The composition of claim 38, wherein said non-human mammal is a SCID mouse.

40. The composition of any one of claims 32 to 39, wherein said neovasculature is human and wherein said in vivo host is non-human, and wherein said cells analyzed with fluorescence digital imaging demonstrate said neovasculature is human and has characteristics that distinguish said neovasculature from non-human host.

41. A method that demonstrates neovasculature formed in vivo has characteristics that distinguish said neovasculature from in vivo host, comprising producing a composition of endothelial cells according to claim 32; expressing in said cells a transformed genetic marker detectable by a digital imaging system; and analyzing said cells with a digital imaging system so as to detect said genetic marker.

42. The method of claim 41, wherein said digital imaging system is a fluorescence digital imaging system and said genetic marker is enhanced green fluorescent protein (eGFP).

43. An isolated graft comprising the cells of claim 1, wherein said cells form microvascular structures in response to a pharmaceutically acceptable compound that modulates angiogenesis.

44. A method for treating atherosclerosis by implanting the graft of claim 43.

45. A method for treating tumors by implanting the graft of claim 43.

46. A method to enhance wound healing by implanting the graft of claim 43.
